(19) United States
(12) Patent Application Publication
(10) Pub. No.: US 2007/0043024 A1
(43) Pub. Date: Feb. 22, 2007

(54) Azacyclic compounds as inhibitors of sensory neurone specific channels

(76) Inventors: Richard John Hamlyn, Cambridge (GB); Michael Richard Huckstep, Cambridge (GB); Rosemary Lynch, Cambridge (GB); Stephen Stokes, Cambridge (GB); David Christopher Tickle, Cambridge (GB); Lee Patient, Cambridge (GB)

Correspondence Address: NIXON & VANDERHYE, PC, 901 NORTH GLEBE ROAD, 11TH FLOOR, ARLINGTON, VA, 22203, US

(21) Appl. No.: 10/563,561

(22) PCT Filed: Jul. 7, 2004
(86) PCT No.: PCT/GB04/02945

§ 371 Date: Jun. 19, 2006

Related U.S. Application Data
(60) Provisional application No. 60/488,442, filed on Jul. 21, 2003.

(30) Foreign Application Priority Data
Jul. 7, 2003 (GB) 0315872.2

(51) Int. Cl. A61L 9/04; A61K 31/55; A61K 31/47; A61K 31/4745

(52) U.S. Cl. 514/215; 514/217.01; 514/300; 514/311; 514/415; 424/45; 540/577; 540/593; 546/122; 546/166; 546/112; 548/496

(57) ABSTRACT

Compounds of the formula (I), and pharmaceutically acceptable salts thereof, are found to be antagonists of SNS sodium channels. They are therefore useful as analgesic and neuroprotective agents wherein: X is —N— or —CH—; n is from 0 to 3.

The present invention relates to inhibitors of the subtype of mammalian sodium channels known as Naᵥ1.8 or sensory neurone specific (SNS) channels. The Naᵥ1.8 channel is a 1,957 amino acid tetrodotoxin-insensitive voltage-gated sodium channel. The sodium channel, nucleic acid sequences coding for the channel, vectors, host cells and methods of identifying modulators, are taught in U.S. Pat. No. 6,451,554. The α-subunit gene corresponding to this ion channel is referred to as SCN10A. The channel is described in more detail in Akopian et al., (1996), 379, 257-262.

Mammalian ion channels are becoming increasingly well characterized, and progress in sodium channel research has been summarized recently in Anger et al, J. Med. Chem. (2001) 44, 115-137. Sodium channels are recognised as valid targets for pain therapeutics, and blockade of sodium channels can be useful in the treatment of a range of pain syndromes (see for example Black et al, Progress in Pain Research and Management (2001), 21 (Neuropathic Pain: Pathophysiology and Treatment), 19-36).

It has now surprisingly been found that compounds of the general formula (I) set out below act as inhibitors of sensory neurone specific sodium channels. Accordingly, the present invention provides a compound of the formula (I), or a pharmaceutically acceptable salt thereof, 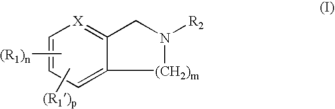
wherein:

X is —N— or —CH—;

n is from 0 to 3;

each R₁ is the same or different and is a hydroxy, amino, halogen, C₁-C₆ alkyl, C₁-C₆ haloalkyl, C₁-C₆ alkoxy, C₂-C₆ alkenyloxy, C₂-C₆ alkynyloxy, C₁-C₆ haloalkoxy, C₁-C₆ alkylthio, C₁-C₆ haloalkylthio, (C₁C₆ alkyl)amino or di(C₁-C₆ alkyl)amino group;

p is 0 or 1;

R₁^{/} is cyano, —NR_{/}—CO—(C₁-C₄ alkyl), —NR_{/}—S(O)₂—(C₁-C₄ alkyl), —CO₂H, —S(O)₂OH, —CO₂—(C₁-C₄ alkyl), —O—S(O)₂—(C₁-C₄ alkyl) or —N[S(O)₂—(C₁-C₄ alkyl)]₂, wherein R_{/} is hydrogen or a C₁-C₄ alkyl group;

m is 1, 2 or 3; and

R₂ is either
(a) -L-A, wherein L is a direct bond or a C₁-C₆ alkyl, C₂-C₆ alkenyl or C₂-C₆ alkynyl moiety and A is C₆-C₁₀ aryl, C₃-C₆ carbocyclyl, a 5- to 10-membered heteroaryl group or a 5- to 10-membered heterocyclic group,
(b) -L-CR(A)₂ or -L-CH═C(A)₂ wherein R is hydrogen or C₁-C₄ alkyl, L is as defined above and each A is the same or different and is as defined above,
(c) -L^{/}-Het-A^{/}, wherein Het is —O—, —S— or —NR^{/}—, A^{/} is -L-A, -L-CR(A)₂ or -L-CH═C(A)₂, R^{/} is H or -L-A, L^{/} is a C₁-C₆ alkyl, C₂-C₆ alkenyl or C₂-C₆ alkynyl moiety, L is as defined above, R is as defined above and each A is the same or different and is as defined above,
(d) -L-CO—NR₃R₄ or -L-CS—NR₃R₄, wherein L is as defined above and either (i) R₃ and R₄, together with the N atom to which they are attached, form a 5- to 10-membered heteroaryl or heterocyclyl group or (ii) R₃ represents -L-H or A^{/} wherein L and A^{/} are as defined above, and R₄ represents -L^{/}-H, -L^{/}-CO-A^{/}, -L^{/}-S(O)-A^{/}, -L^{/}-S(O)₂-A^{/}, -L^{/}-Het-A^{/}, —NR—CO—N(A)₂, —N(A)₂, -A-Het-A, -A^{/}, -L-CR(LA)₂ or -L-CH═C(LA)₂ wherein each L is the same or different, each A is the same or different, and L^{/}, L, R, A and A^{/} are as defined above,
(e) —CO-L-NR₃R₄ or —CS-L-NR₃R₄ wherein L, R₃ and R₄ are as defined above,
(f) —CO-A^{/} or —CS-A^{/} wherein A^{/} is as defined above,
(g) -L^{/}-O—N═C(A)₂ or —CO-L^{/}-O—N═C(A)₂ wherein L^{/} is as defined above and each A is the same or different and is as defined above, or
(h) -L^{/}-NR—CO—NR₃R₄ or -L^{/}-NR—CS—NR₃R₄, wherein L^{/}, R, R₃ and R₄ are as defined above,
   wherein

said aryl, carbocyclyl, heteroaryl and heterocyclyl groups are optionally fused to one or two cyclic moieties selected from phenyl rings and 5- to 6-membered heterocyclyl and heteroaryl groups, and

said aryl, heteroaryl, carbocyclyl and heterocyclyl groups are unsubstituted or are substituted by 1, 2 or 3 substituents which are the same or different and are selected from C₁-C₄ alkyl, C₁-C₄ haloalkyl, halogen, hydroxy, amino, (C₁-C₄ alkyl)amino, di(C₁-C₄ alkyl)amino, C₁-C₄ alkoxy, C₁-C₄ haloalkoxy, C₁-C₄ alkylthio, C₁-C₄ haloalkylthio, —NH—CO—(C₁-C₄ alkyl), —CO—(C₁-C₄ alkyl), —CO₂—(C₁-C₄ alkyl), 5- or 6-membered heteroaryl, phenyl and —CHPh₂ substituents, the phenyl and heteroaryl moieties in said substituents being unsubstituted or substituted by 1 or 2 further substituents selected from halogen atoms, C₁-C₂ alkyl groups, C₁-C₂ alkoxy groups and —NH—CO—(C₁-C₂ alkyl) groups,

provided that (a) when R₂ is -L-A, A is other than a benzimidazolyl group, and (b) when R₂ is —CO-A^{/} or —CS-A^{/}, A is other than a pyrazolopyrimidinyl or pyrazolyl group.

Typically, the compounds of the invention are compounds of formula (I), and pharmaceutically acceptable salts thereof, wherein:

X is —N— or —CH—;

n is from 0 to 3;

p is 0;

each R₁ is the same or different and is a hydroxy, amino, halogen, C₁-C₆ alkyl, C₁-C₆ haloalkyl, C₁-C₆ alkoxy, C₁-C₆ haloalkoxy, C₁-C₆ alkylthio, C₁-C₆ haloalkylthio, (C₁C₆ alkyl)amino or di(C₁-C₆ alkyl)amino group;

m is 1, 2 or 3; and

R₂ is either
(a) -L-A, wherein L is a direct bond or a C₁-C₆ alkyl, C₂-C₆ alkenyl or C₂-C₆ alkynyl moiety and A is C₆-C₁₀ aryl, C₃-C₆ carbocyclyl, a 5- to 10-membered heteroaryl group or a 5- to 10-membered heterocyclic group,
(b) -L-CR(A)₂ or -L-CH═C(A)₂ wherein R is hydrogen or C₁-C₄ alkyl, L is as defined above and each A is the same or different and is as defined above,
(c) -L^{/}-Het-A^{/}, wherein Het is —O—, —S— or —NR^{/}—, A^{/} is -L-A, -L-CR(A)₂ or -L-CH═C(A)₂, R^{/} is H or -L-A, L^{/} is a C₁-C₆ alkyl, C₂-C₆ alkenyl or C₂-C₆ alkynyl moiety, L is as defined above, R is as defined above and each A is the same or different and is as defined above,
(d) -L-CO—NR₃R₄ or -L-CS—NR₃R₄, wherein L is as defined above and either (i) R₃ and R₄, together with the N atom to which they are attached, form a 5- to 10-membered heteroaryl or heterocyclyl group or (ii) R₃ represents -L-H or A^{/} wherein L and A^{/} are as defined above, and R₄ represents -L^{/}-H, -L^{/}-CO-A, A^{/}, -L-CR(LA)₂ or -L-CH═C(LA)₂ wherein each L is the same or different, each A is the same or different, and L^{/}, L, R, A and A^{/} are as defined above,
(e) —CO-L-NR₃R₄ or —CS-L-NR₃R₄ wherein L, R₃ and R₄ are as defined above,
(f) —CO-A^{/} or —CS-A^{/} wherein A^{/} is as defined above, or
(g) -L^{/}-O—N═C(A)₂ or —CO-L^{/}-O—N═C(A)₂ wherein L^{/} is as defined above and each A is the same or different and is as defined above,
   wherein

said aryl, carbocyclyl, heteroaryl and heterocyclyl groups are optionally fused to one or two cyclic moieties selected from phenyl rings and 5- to 6-membered heterocyclyl and heteroaryl groups, and

said aryl, heteroaryl, carbocyclyl and heterocyclyl groups are unsubstituted or are substituted by 1, 2 or 3 substituents which are the same or different and are selected from C₁-C₄ alkyl, C₁-C₄ haloalkyl, halogen, hydroxy, C₁-C₄-alkoxy, C₁-C₄ haloalkoxy, C₁-C₄ alkylthio, C₁-C₄ haloalkylthio, phenyl and —CHPh₂ substituents, the phenyl moieties in said substituents being unsubstituted or substituted by 1 or 2 halogen atoms,

provided that (a) when R₂ is -L-A, A is other than a benzimidazolyl group and (b) when R₂ is —CO-A^{/} or —CS-A^{/}, A is other than a pyrazolopyrimidinyl or pyrazolyl group.

As used herein, a C₁-C₆ alkyl group or moiety is a linear or branched alkyl group or moiety containing from 1 to 6 carbon atoms, such as C₁-C₄ alkyl group or moiety, for example methyl, ethyl, n-propyl, i-propyl, n-butyl, i-butyl and t-butyl. A divalent alkyl moiety (or alkylene moiety) can be attached via the same carbon atom, by adjacent carbon atoms or by non-adjacent carbon atoms.

As used herein, a C₂-C₆ alkenyl group or moiety is a linear or branched alkenyl group or moiety containing from 2 to 6 carbon atoms, such as a C₂-C₄ alkenyl group or moiety, for example ethenyl, propenyl and butenyl. Typically, an alkenyl group or moiety is saturated except for one double bond. A divalent alkenyl moiety (or alkenylene moiety) can be attached via the same carbon atoms, via adjacent carbon atoms or via non-adjacent carbon atoms.

As used herein, a C₂-C₆ alkynyl group or moiety is a linear or branched alkynyl group or moiety containing from 2 to 6 carbon atoms, such as a C₂-C₄ alkynyl group or moiety, for example ethynyl, propynyl and butynyl. Typically, an alkynyl group or moiety is saturated except for one triple bond. A divalent alkynyl moiety (or alkynylene moiety) can be attached via the same carbon atom, via adjacent carbon atoms or via non-adjacent carbon atoms.

As used herein, a C₆-C₁₀ aryl group or moiety is typically a phenyl or naphthyl group or moiety. It is preferably a phenyl group or moiety.

As used herein, a 5- to 10-membered heteroaryl group is a 5- to 10-membered aromatic ring, such as a 5- or 6-membered ring, containing at least one heteroatom, for example 1, 2 or 3 heteroatoms, selected from O, S and N. Examples include pyridyl, pyrazinyl, pyrimidinyl, pyridazinyl, furanyl, thienyl, imidazolyl, pyrazolidinyl, pyrrolyl, oxadiazolyl, isoxazyl, thiadiazolyl, thiazolyl and pyrazolyl groups. Thienyl, triazolyl, pyridyl, thiazolyl and imidazolyl groups are preferred. Pyrrolyl groups are also preferred.

As used herein, a halogen is typically chlorine, fluorine, bromine or iodine and is preferably chlorine or fluorine. As used herein, a said C₁-C₆ alkoxy group is typically a said C₁-C₆ alkyl group attached to an oxygen atom. A said C₁-C₆ alkylthio group is typically a said C₁-C₆ alkyl group attached to a thio group.

As used herein, a C₁-C₆ haloalkyl group is typically a said C₁-C₆ allyl group, for example a C₁-C₄ alkyl group, substituted by one or more said halogen atoms. Typically, it is substituted by 1, 2 or 3 said halogen atoms. Preferred haloalkyl groups include perhaloalkyl groups such as —CX₃ wherein X is a said halogen atom. Particularly preferred haloalkyl groups are —CF₃ and —CCl₃.

As used herein, a C₁-C₆ haloalkoxy group is typically a said C₁-C₆ alkoxy group, for example a C₁-C₄ alkoxy-group, substituted by one or more said halogen atoms. Typically, it is substituted by 1, 2 or 3 said halogen atoms. Preferred haloalkoxy groups include perhaloalkoxy groups such as —OCX₃ wherein X is a said halogen atom. Particularly preferred haloalkoxy groups are —OCF₃ and —OCCl₃.

As used herein, a C₁-C₆ haloalkylthio group is typically a said C₁-C₆ alkylthio group, for example a C₁-C₄ alkylthio group, substituted by one or more said halogen atoms. Typically, it is substituted by 1, 2 or 3 said halogen atoms. Preferred haloalkylthio groups include perhaloalkylthio groups such as —SCX₃ wherein X is a said halogen atom. Particularly preferred haloalkylthio groups are —SCF₃ and —SCCl₃.

As used herein, a C₃-C₆ carbocyclyl group or moiety is a non-aromatic saturated or unsaturated hydrocarbon ring, having from 3 to 6 carbon atoms.

Preferably it is a saturated group, i.e. a C₃-C₆ cycloalkyl group. Examples include cyclobutyl, cyclopentyl and cyclohexyl.

As used herein, a 5- to 10-membered heterocyclyl group or moiety is a non-aromatic, saturated or unsaturated C₅-C₁₀ carbocyclic ring in which one or more, for example 1, 2 or 3, of the carbon atoms are replaced by a moiety selected from N, O, S, S(O) and S(O)₂. Preferably, only one carbon atom is replaced with a —S(O)— or —S(O)₂— moiety. More preferably, a 5- to 10-membered heterocyclyl group or moiety is a non-aromatic, saturated or unsaturated C₅-C₁₀ carbocyclic ring in which one or more, for example 1, 2 or 3, of the carbon atoms are replaced by a heteroatom selected from N, O and S.

Saturated heterocyclyl groups are preferred. Examples of suitable heterocyclyl groups include piperidinyl, piperazinyl, morpholinyl, pyrrolidinyl, tetrahydrofuranyl, imidazolidinyl, thiazolidinyl, 1,4 dioxanyl, 1,3 dioxolanyl and homopiperidinyl groups. Further examples of suitable heterocyclyl groups include thiomorpholino, S-oxo-thiomorpholino and S,S-dioxo-thiomorpholino groups. Preferred heterocyclyl groups are piperidinyl, morpholinyl, piperazinyl and homopiperidinyl groups. Further preferred heterocyclyl groups are thiomorpholino, S-oxo-thiomorpholino and S,S-dioxo-thiomorpholino groups.

Typically, when a said aryl, carbocyclyl, heteroaryl or heterocyclyl group is fused to two cyclic moieties selected from phenyl rings and 5- to 6-membered heterocyclyl and heteroaryl groups, said cyclic moieties are fused directly to the aryl, carbocyclyl, heteroaryl or heterocyclyl group. Typically, the two cyclic moieties are not fused together.

Preferably 0, 1 or 2 of the said substituents on an aryl, heteroaryl, carbocyclyl or heterocyclyl group are selected from —NH—CO—(C₁-C₄ alkyl), —CO—(C₁-C₄ alkyl), —CO₂—(C₁-C₄ alkyl), 5- or 6-membered heteroaryl, phenyl and —CHPh₂ substituents.

Typically, the aryl, heteroaryl, heterocyclyl and carbocyclyl groups and moieties in the substituents R₁, R₂, R₃ and R₄ are unsubstituted or are substituted by 1, 2 or 3 substituents which are the same or different and are selected from halogen, C₁-C₄ alkyl, hydroxy, amino, (C₁-C₄ alkyl)amino, C₁-C₄ alkoxy, C₁-C₄ haloalkyl, C₁-C₄ haloalkoxy, C₁-C₄ alkylthio, C₁-C₄ haloalkylthio, —NH—CO—(C₁-C₂ alkyl), —CO—(C₁-C₂ alkyl), —CO₂—(C₁-C₂ alkyl), 5-membered heteroaryl, phenyl and —CHPh₂ substituents, the phenyl and heteroaryl moieties in said substituents being unsubstituted or substituted by 1 or 2 further substituents selected from halogen atoms, C₁-C₂ alkyl groups, C₁-C₂ alkoxy groups and —NH—CO—(C₁-C₂ alkyl) groups. More typically, the above substituents are selected from halogen, C₁-C₄ alkyl, hydroxy, C₁-C₄ alkoxy, C₁-C₄ haloalkyl, C₁-C₄ haloalkoxy, C₁-C₄ alkylthio, C₁-C₄ haloalkylthio, phenyl and —CHPh₂ substituents, the phenyl moieties in said substituents being unsubstituted or substituted by 1 or 2 halogen atoms.

Preferably, the aryl, heteroaryl, heterocyclyl and carbocyclyl groups and moieties in the substituents R₁, R₂, R₃ and R₄ are unsubstituted or are substituted by 1 or 2 substituents which are the same or different and are selected from halogen, C₁-C₄ alkyl, hydroxy, amino, C₁-C₂ alkoxy, C₁-C₂ haloalkyl, C₁-C₂ haloalkoxy, C₁-C₂ haloalkylthio, —NH—CO—(C₁-C₂ alkyl), —CO—(C₁-C₂ alkyl), —CO₂—(C₁-C₂ alkyl), oxadiazolyl, phenyl and —CHPh₂ substituents, the oxadiazolyl and phenyl moieties in said substituents being unsubstituted or substituted by 1 or 2 further substituents selected from halogen atoms, methyl groups, methoxy groups and —NH—CO—CH₃ groups. Preferably, these preferred substituents are selected from halogen, C₁-C₂ alkyl, hydroxy, C₁-C₂ alkoxy, C₁-C₂ haloalkyl, C₁-C₂ haloalkoxy, C₁-C₂ haloalkylthio, phenyl and —CHPh₂ substituents, the phenyl moieties in said substituents being unsubstituted or substituted by 1 or 2 further substituents selected from fluorine and chlorine atoms.

Typically, X is —CH—.

Typically, n is 0 or 1.

Preferably, each R₁ is the same or different and is a hydroxy, amino, halogen, C₁-C₄ alkyl, C₁-C₄ haloalkyl, C₁-C₄ alkoxy, C₂-C₄ alkenyloxy, C₁-C₄ haloalkoxy, C₁-C₄ alkylthio or C₁-C₄ haloalkylthio group. Typically, in this preferred embodiment each R₁ is the same or different and is a hydroxy, halogen, C₁-C₄ alkyl, C₁-C₄ haloalkyl, C₁-C₄ alkoxy, C₁-C₄ haloalkoxy, C₁-C₄ alkylthio or C₁-C₄ haloalkylthio group.

More preferably, each R₁ is the same or different and is C₁-C₂ alkyl, C₂-C₃ alkenyloxy, amino, hydroxy or C₁-C₂ alkoxy. Typically, in this more preferred embodiment each R₁ is the same or different and is C₁-C₂ alkyl, hydroxy or C₁-C₂ alkoxy.

Typically, R₁^{/} is cyano, —NH—CO—(C₁-C₄ alkyl), —NH—S(O)₂—(C₁-C₄ alkyl), —O—S(O)₂—(C₁-C₄ alkyl), —S(O)₂—OH or —N—[S(O)₂—(C₁-C₄ alkyl)]₂. Preferably, R₁^{/} is cyano, —NH—CO—CH₃, —NH—S(O)₂—CH₃, —O—S(O)₂—CH₃, —N—[SO₂—CH₃]₂ or —S(O)₂OH.

Typically p is 0 and R₁ is located meta to the fused heterocycle, or on the phenyl carbon atom nearest the N atom. Thus, the compound of formula (I) is typically a compound of formula

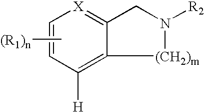

Typically, each L moiety in the R₂ substituent is the same or different and represents a direct bond or a C₁-C₆ alkyl moiety. Preferably, each L is the same or different and represents a direct bond or a C₁-C₄ alkyl moiety, for example a methyl, ethyl or propyl moiety, for example —CH(CH₃)— or —CH₂—CH(CH₃)—.

Typically each L^{/} moiety in the R₂ substituent is the same or different and represents a C₁-C₆ alkyl moiety, preferably a C₁-C₄ alkyl moiety, for example a methyl, ethyl or propyl moiety, for example —CH(CH₃)— or —CH₂—CH(CH₃)—.

Typically, each A moiety in the R₂ substituent is the same or different and represents a C₆-C₁₀ aryl, C₃-C₆ cycloalkyl, 5- or 6-membered heterocyclyl or 5- or 6-membered heteroaryl group, which group is (a) unsubstituted or substituted by 1, 2 or 3 substituents selected from C₁-C₄ alkyl, C₁-C₄ haloalkyl, halogen, hydroxy, amino, (C₁-C₄ alkyl)amino, di(C₁-C₄ alkyl)amino, C₁-C₄ alkoxy, C₁-C₄ haloalkoxy, C₁-C₄ alkylthio, C₁-C₄ haloalkylthio, —NH—CO—(C₁-C₂ alkyl), phenyl and halophenyl substituents and (b) optionally fused to one or two cyclic moieties selected from phenyl rings and 5- to 6-membered heterocyclyl or heteroaryl groups. For the avoidance of doubt, said preferred substituents on the moiety A are themselves unsubstituted.

More typically, each A moiety in the R₂ substituent is the same or different and represents a C₆-C₁₀ aryl, C₃-C₆ cycloalkyl, 5- or 6-membered heterocyclyl or 5- or 6-membered heteroaryl group, which group is (a) unsubstituted or substituted by 1, 2 or 3 substituents selected from C₁-C₄ alkyl, C₁-C₄ haloalkyl, halogen, hydroxy, C₁-C₄ alkoxy, C₁-C₄ haloalkoxy, C₁-C₄ alkylthio, C₁-C₄ haloalkylthio, phenyl and halophenyl substituents and (b) optionally fused to one or two cyclic moieties selected from phenyl rings and 5- to 6-membered heterocyclyl or heteroaryl groups.

Further, each A moiety in the R₂ substituent is typically the same or different and is a phenyl, thienyl, triazolyl, pyridyl, pyrrolyl, pyrrolidinyl, 4-H-pyranyl, cyclopentyl, imidazolyl, thiazolyl or piperidyl group which is (a) unsubstituted or substituted by one or two substituents selected from halogen, C₁-C₂ haloalkyl, C₁-C₂ haloalkoxy, C₁-C₂ haloalkylthio, phenyl, C₁-C₂ alkyl, C₁-C₂ alkoxy, amino, hydroxy and —NH—CO—(C₁-C₂ alkyl) groups and (b) optionally fused to one or two cyclic moieties selected from phenyl rings and 5- to 6-membered heteroaryl moieties. More typically, each A moiety in the R₂ substituent is the same or different and is a phenyl, thienyl, triazolyl, pyridyl, cyclopentyl, imidazolyl, thiazolyl or piperidyl group which is (a) unsubstituted or substituted by one or two substituents selected from halogen, C₁-C₂ haloalkyl, C₁-C₂ haloalkoxy, C₁-C₂ haloalkylthio, phenyl, C₁-C₂ alkyl, C₁-C₂ alkoxy and hydroxy groups and (b) optionally fused to one or two cyclic moieties selected from phenyl rings and 5- to 6-membered heteroaryl moieties.

Preferably, each A moiety in the R₂ substituent is a phenyl, thienyl, triazolyl, pyridyl, fluorenyl, thiazolyl, tetrahydroisoquinolinyl, 9H-carbazolyl, indolinyl, 9H-xanthenyl or benzimidazolyl group, which group is unsubstituted or substituted by one or two substituents selected from halogen, C₁-C₂ alkyl, hydroxy, amino, C₁-C₂ alkoxy, C₁-C₂ haloalkyl, C₁-C₂ haloalkoxy, C₁-C₂ haloalkylthio, —NH—CO—CH₃ and phenyl substituents. More typically, in this preferred embodiment, each A moiety is a phenyl, thienyl, triazolyl, pyridyl, fluorenyl, thiazolyl, tetrahydroisoquinolinyl or benzimidazolyl group, which group is unsubstituted or substituted by one or two substituents selected from halogen, C₁-C₂ alkyl, hydroxy, C₁-C₂ alkoxy, C₁-C₂ haloalkyl, C₁-C₂ haloalkoxy, C₁-C₂ haloalkylthio and phenyl substituents.

Typically, each R substituent in each —CR(A)₂ moiety is the same or different and is hydrogen or methyl.

Typically, each Het moiety in the R₂ substituent is —O—, —S— or —NR^{/}— wherein R^{/} is hydrogen, C₁-C₄ alkyl, phenyl or —(C₁-C₄ alkyl)-phenyl. More preferably, each Het moiety in the R₂ substituent is —O— or —NR^{/}— wherein R^{/} is hydrogen, C₁-C₄ alkyl or benzyl.

When R₃ and R₄, together with the N atom to which they are attached, form a heteroaryl or heterocyclyl group, the heteroaryl or heterocyclyl group is typically (a) monocyclic, (b) fused to one or two phenyl rings or (c) a morpholino group which is fused to a phenyl ring and to a 1H-pyrazolyl group.

Typically, when R₃ and R₄, together with the N atom to which they are attached, form a heterocycle, they form a 5- to 7-membered heterocyclyl group. Preferably, they form a morpholino, thiomorpholino, S-oxo-thiomorpholino, S,S-dioxo-thiomorpholino, pyrrolidinyl, piperazinyl or homopiperidinyl ring which is (a) optionally fused to one or two cyclic moieties selected from phenyl rings and 5- to 6-membered heteroaryl rings, and (b) unsubstituted or substituted by 1 or 2 substituents selected from C₁-C₄ alkyl, C₁-C₄ haloalkyl, C₁-C₄ alkoxy, C₁-C₄ alkylthio, halogen, phenyl, —CHPh₂, —CO—(C₁-C₂ alkyl); —CO₂—(C₁-C₂ alkyl) and 5- to 6-membered heteroaryl substituents, the phenyl and heteroaryl moieties in said substituents being unsubstituted or substituted by 1 or 2 further substituents selected from halogen atoms, C₁-C₂ alkyl groups, C₁-C₂ alkoxy groups and —NH—CO—(C₁-C₂ alkyl) groups.

More typically, when R₃ and R₄, together with the N atom to which they are attached, form a heterocycle, they form a morpholino, piperazinyl or homopiperidinyl ring which is (a) unsubstituted or substituted by 1 or 2 substituents selected from C₁-C₄ alkyl, C₁-C₄ haloalkyl, halogen, phenyl and —CHPh₂ substituents, the phenyl moieties in said substituents being unsubstituted or substituted by 1 or 2 halogen atoms and (b) optionally fused to one or two phenyl rings.

Typically, when R₃ and R₄ do not together form a heterocycle, R₃ represents hydrogen, C₁-C₄ alkyl, phenyl, —(C₁-C₄ alkyl)-phenyl or —(C₁-C₄ alkyl)-CHPh₂. More typically, when R₃ and R₄ do not together form a heterocycle, R₃ represents hydrogen, C₁-C₄ alkyl, —(C₁-C₄ alkyl)-phenyl or —(C₁-C₄ alkyl)-CHPh₂. Preferably, the phenyl moieties in R₃ are unsubstituted or substituted by a hydroxy group. More preferably, R₃ is unsubstituted.

More preferably, R₃ represents hydrogen, C₁-C₄ alkyl or an unsubstituted benzyl, phenyl, hydroxyphenyl or —(C₁-C₂ alkyl)-CHPh₂ group. Most preferably R₃ represents hydrogen, C₁-C₄ alkyl or an unsubstituted benzyl or —(C₁-C₂ alkyl)-CHPh₂ group.

Typically, when R₃ and R₄ do not together form a heterocycle, R₄ represents C₁-C₄ alkyl, A, —(C₁-C₄ alkyl)-A, —(CH₂)ₘ—CH(A)₂, —CH[(CH₂)ₘA]₂, —(CH₂)ₘ—CO-A, —(CH₂)—O—CH(A)₂, —(CH₂)ₘ—S—CH(A)₂, —(CH₂)ₘ—S(O)—CH(A)₂, —(CH₂)ₘ—S(O)₂—CH(A)₂, —NH—CO—N(A)₂, —N(A)₂ or -A-O-A, wherein each A is the same or different and is as defined above and m is 0, 1, 2, 3 or 4. More typically, when R₃ and R₄ do not together form a heterocycle, R₄ represents C₁-C₄ alkyl, A, —(C₁-C₄ alkyl)-A, —(CH₂)ₘ—CH(A)₂, —CH[(CH₂)ₘA]₂ or —(CH₂)ₘ—CO-A wherein each A is the same or different and is as defined above and m is 0, 1, 2, 3 or 4.

Preferably, the A moieties in the R₄ substituent are (a) unsubstituted or substituted by 1 or 2 substituents selected from C₁-C₄ alkyl, C₁-C₄ alkoxy, halogen, hydroxy, amino, C₁-C₂ haloalkyl, C₁-C₂ haloalkoxy and C₁-C₂ haloalkylthio substituents and (b) monocyclic or fused to 1 or 2 phenyl rings. Typically, in this preferred embodiment, the A moieties in the R₄ substituent are (a) unsubstituted or substituted by 1 or 2 substituents selected from C₁-C₄ alkyl, C₁-C₄ alkoxy, halogen, C₁-C₂ haloalkyl, C₁-C₂ haloalkoxy and C₁-C₂ haloalkylthio substituents and (b) monocyclic or fused to 1 or 2 phenyl rings.

More preferably, when R₃ and R₄ do not together form a heterocycle, R₄ represents C₁-C₄ alkyl, fluorenyl, phenyl, pyridyl, —(C₁-C₄ alkyl)-phenyl, —(C₁-C₄ alkyl)-(5- to 6-membered heteroaryl), —(CH₂)ₘ-(9H-carbazolyl), —(CH₂)ₘ-indolinyl, —(CH₂)ₘ-(9H-xanthenyl), —(CH₂)ₘ—O—CHA^{//}A^{///}, —(CH₂)ₘS—CHA^{//}A^{///}, —(CH₂)ₘ—S(O)—CHA^{//}A^{///}, —(CH₂)ₘ—S(O)₂—CHA^{//}A^{///}, —NH—CO—N(phenyl)₂, —N(Phenyl)₂, -A^{//}-O-A^{///}, —(CH₂)ₘ—CHA^{//}A^{///}, —CH[(CH₂)ₙPh]₂ or —(CH₂)ₚ—CO—R, wherein m is 0, 1, 2 or 3, A^{//} and A^{///} are the same or different and each represent phenyl or a 5- or 6-membered heteroaryl group, n is 0, 1 or 2, p is 1, 2 or 3 and R is a 5- or 6-membered heterocyclic group fused to a phenyl ring, for example a tetrahydroisoquinoline group, the cyclic moieties in said preferred R₄ groups being unsubstituted or substituted by a halogen atom, C₁-C₂ alkyl, hydroxy, amino or C₁-C₂ alkoxy group.

More preferably, when R₃ and R₄ do not together form a heterocycle, R₄ represents C₁-C₄ alkyl, fluorenyl, —(C₁-C₄ alkyl)-phenyl, —(C₁-C₄ alkyl)-(5- to 6-membered heteroaryl), —(CH₂)ₘ—CHA^{//}A^{///} wherein m is 0, 1, 2 or 3 and A^{//} and A^{///} are the same or different and each represent phenyl or a 5- or 6-membered heteroaryl group, —CH[(CH₂)ₙPh]₂ wherein n is 0, 1 or 2, or —(CH₂)ₚ—CO—R wherein p is 1, 2 or 3 and R is a 5- or 6-membered heterocyclic group fused to a phenyl ring, for example a tetrahydroisoquinoline group, the cyclic moieties in said most preferred R₄ groups being unsubstituted or substituted by a halogen atom, C₁-C₂ alkyl or C₁-C₂ alkoxy group.

Typically, when R₂ is defined according to option (a), A is monocyclic. More typically, A is a monocyclic phenyl or 5- to 6-membered heteroaryl group.

Typically, when R₂ is defined according to option (a), L is C₁-C₄ alkyl and A is a phenyl or 5- or 6-membered heteroaryl group, which group is unsubstituted or substituted by 1, 2 or 3 substituents selected from C₁-C₄ alkyl, C₁-C₄ haloalkyl, halogen, hydroxy, C₁-C₄ alkoxy, C₁-C₄ haloalkoxy, C₁-C₄ alkylthio, C₁-C₄ haloalkylthio, phenyl and halophenyl substituents.

Preferably, when R₂ is defined according to option (a), it is a —(C₁-C₄ alkyl)-phenyl group, for example benzyl, or a —(C₁-C₄ alkyl)-(5- to 6-membered heteroaryl) group, for example —CH₂-thienyl or —CH₂-triazolyl, the phenyl and heteroaryl moieties being unsubstituted or substituted by 1 or 2 substituents selected from C₁-C₂ haloalkyl, halogen, C₁-C₂ haloalkylthio, C₁-C₂ haloalkoxy, C₁-C₂ alkyl and phenyl substituents.

Typically, when R₂ is defined according to option (b), it is -L-CR(A)₂ wherein R and A are as defined above. Preferably, L is C₁-C₄ alkyl, R is hydrogen or methyl and each A is the same or different and is a phenyl group which is unsubstituted or substituted by 1, 2 or 3 substituents selected from halogen, C₁-C₂ haloalkyl, C₁-C₂ alkyl, —NH—CO—CH₃ and hydroxy substituents. More preferably, L is C₁-C₄ alkyl, R is hydrogen or methyl and each A is the same or different and is a phenyl group which is unsubstituted or substituted by 1, 2 or 3 substituents selected from halogen, C₁-C₂ haloalkyl, C₁-C₂ alkyl and hydroxy substituents.

Typically, when R₂ is defined according to option (c), L^{/} is C₁-C₄ alkyl, Het is O, NH or —N(benzyl)- and A^{/} is an unsubstituted —(C₁-C₄)alkyl-phenyl, —(C₁-C₄ alkyl)-CHPh₂ or —CH═CHPh₂ group.

Typically, when R₂ is defined according to option (d), L is other than a direct bond. More typically, L is C₁-C₆ alkyl.

Further, when R₂ is defined according to option (d), it is typically -L-CO—NR₃R₄. More typically, when R₂ is defined according to option (d), R₂ is —(CH₂)_{q}—CO—NR₃R₄ wherein q is from 1 to 4, and is preferably 1 or 2, and R₃ and R₄ are as defined above.

Preferably, when R₂ is defined according to option (d), either (i) R₃ and R₄, together with the N atom to which they are attached, form a 5- to 7-membered heterocyclyl group or (ii) R₃ represents hydrogen, C₁-C₄ alkyl, phenyl or —(C₁-C₄ alkyl)-phenyl and R₄ represents C₁-C₄ alkyl, A, —(C₁-C₄ alkyl)-A, —(CH₂), —CH(A)₂, —CH[(CH₂)ₘA]₂, —(CH₂)ₘ—CH(A)₂, —(CH₂)ₘ—S—CH(A)₂, —(CH₂)ₘ—S(O)—CH(A)₂, —(CH₂)ₘ—S(O)₂—CH(A)₂, —NH—CO—N(A)₂, —N(A)₂ or -A-O-A, wherein each A is the same or different and is as defined above and m is 0, 1, 2, 3 or 4. Typically, in this preferred embodiment when R₂ is defined according to option (d), either (i) R₃ and R₄, together with the N atom to which they are attached, form a 5- to 7-membered heterocyclyl group or (ii) R₃ represents hydrogen, C₁-C₄ alkyl or —(C₁-C₄ alkyl)-phenyl and R₄ represents C₁-C₄ alkyl, A, —(C₁-C₄ alkyl)-A, —(CH₂)ₘ—CH(A)₂ or —CH[(CH₂)ₘA]₂ wherein each A is the same or different and is as defined above and m is 0, 1, 2, 3 or 4.

More preferably, when R₂ is defined-according to option (d) either (i) R₃ and R₄, together with the N atom to which they are attached, form a morpholino, thiomorpholino, S-oxo-thiomorpholino, S,S-dioxo-thiomorpholino, pyrrolidinyl, piperazinyl or homopiperdinyl ring which is (a) optionally fused to 1 or 2 cyclic moieties selected from phenyl rings and 5- to 6-membered heteroaryl rings and (b) unsubstituted or substituted by 1 or 2 substituents selected from C₁-C₄ alkyl, C₁-C₄ haloalkyl, C₁-C₄ alkoxy, C₁-C₄ alkylthio, halogen, phenyl, —CHPh₂, —CO—(C₁-C₂ alkyl), —CO₂—(C₁-C₂ alkyl) and 5- to 6-membered heteroaryl substituents, the phenyl and heteroaryl moieties in said substituents being unsubstituted or substituted by 1 or 2 further substituents selected from halogen atoms, C₁-C₂ alkyl groups, C₁-C₂ alkoxy groups and —NH—CO—(C₁-C₂ alkyl) groups or (ii) R₃ represents hydrogen, C₁-C₄ alkyl or an unsubstituted benzyl, phenyl or hydroxyphenyl group and R₄ represents C₁-C₄ alkyl, fluorenyl, phenyl, pyridyl, —(C₁-C₄ alkyl)-phenyl, —(C₁-C₆ alkyl)-(5- to 6-membered heteroaryl), —(CH₂)ₘCHA^{//}A^{///}, —CH[(CH₂)ₙPh]₂, —(CH₂)ₘ-(9H-carbazolyl), —(CH₂)ₘ-indolinyl, —(CH₂)ₘ-(9H-xanthenyl), —(CH₂)ₘ—O—CHA^{//}A^{///}, —(CH₂), —S—CHA^{//}A^{///}, —(CH₂)ₘ—S(O)—CHA^{//}A^{///}, —(CH₂)ₘ—S(O)₂—CHA^{//}A^{///}, —NH—CO—N(Phenyl)₂, —N(phenyl)₂ or -A^{//}-O-A^{///}, wherein m is 0, 1, 2 or 3, A^{//} and A^{///} are the same or different and each represent phenyl or a 5- or 6-membered heteroaryl group, and n is 0, 1 or 2, the cyclic moieties in these groups being unsubstituted or substituted by a halogen atom, C₁-C₂ alkyl, hydroxy, amino or C₁-C₂ alkoxy group.

More preferably when R₂ is defined according to option (d) either (i) R₃ and R₄, together with the N atom to which they are attached, form a morpholino, piperazinyl or homopiperdinyl ring which is (a) unsubstituted or substituted by 1 or 2 substituents selected from C₁-C₄ alkyl, C₁-C₄ haloalkyl, halogen, phenyl and —CHPh₂ substituents, the phenyl moieties in said substituents being unsubstituted or substituted by 1 or 2 halogen atoms and (b) optionally fused to one or two phenyl rings or (ii) R₃ represents hydrogen, C₁-C₄ alkyl or an unsubstituted benzyl group and R₄ represents C₁-C₄ alkyl, fluorenyl, —(C₁-C₄ alkyl)-phenyl, —(C₁-C₆ alkyl)-(5- to 6-membered heteroaryl), —(CH₂)ₘCHA^{//}A^{///} wherein m is 0, 1, 2 or 3 and A^{//} and A^{///} are the same or different and each represent phenyl or a 5- or 6-membered heteroaryl group, or —CH[(CH₂)ₙPh]₂ wherein n is 0, 1 or 2, the cyclic moieties in these groups being unsubstituted or substituted by a C₁-C₂ alkyl group.

Typically, when R₂ is defined according to option (e), L is a direct bond or a C₁-C₄ alkyl moiety, for example a methyl moiety, and R₃ and R₄ are as defined above.

Typically, when R₂ is defined according to option (f), A is a said C₆-C₁₀ aryl group. Typically, when R₂ is defined according to option (f), it is —CO-A^{/}. More typically, when R₂ is defined according to option (f), it is —CO-L-CH(A)₂ or —CO-L-A, wherein L is as defined above and each A is the same or different and is as defined above.

Preferably, when R₂ is defined according to option (f), it is —CO—CH₂—CH(R)₂ or —CO—R^{/}, wherein each R is the same or different and is a phenyl or halophenyl moiety and R^{/} is a benzimidazolyl group.

Typically, when R₂ is defined according to option (g), it is —CO-L^{/}-O—N═C(A)₂, wherein L^{/} is as defined above and each A is the same or different and is as defined above. Preferably, when R₂ is defined according to option (g), it is —CO—CH₂—O—N═CR^{//}R^{///} wherein R^{//} and R^{///} are the same or different and each represent an unsubstituted phenyl or pyridyl group.

Typically, when R₂ is defined according to option (h), L^{/} is C₁-C₄ alkyl. Typically, R is H. Typically, either (i) R₃ and R₄, together with the N atom to which they are attached, form a phenothiazine or phenoxazine group or (ii) R₃ is hydrogen and R₄ is —(CH₂)ₘ—CHA^{//}A^{///} or -A^{//}-O-A^{///} wherein m is 0, 1, 2 or 3 and A^{//} and A^{///} are the same or different and each represent phenyl or a 5- to 6-membered heteroaryl group. Preferably, A^{//} and A^{///} are both phenyl.

Preferred compounds of formula (I) are those in which:

X is —N— or —CH—;

n is from 0 to 3;

m is 1, 2 or 3;

each R₁ is the same or different and is a hydroxy, amino, halogen, C₁-C₄ alkyl, C₁-C₄ haloalkyl, C₁-C₄ alkoxy; C₁-C₄ haloalkoxy, C₂-C₄ alkenyloxy C₁-C₄ alkylthio, or C₁-C₄ haloalkylthio group;

p is 0 or 1;

R₁^{/} is cyano, —NH—CO—(C₁-C₄ alkyl), —NH—S(O)₂—(C₁-C₄ alkyl), —O—S(O)₂—(C₁-C₄ alkyl), —S(O)₂—OH or —N[S(O)₂—(C₁-C₄ alkyl]₂; and

R₂ is either
(a) -L-A wherein L is a direct bond or a C₁-C₆ alkyl moiety and A is a C₆-C₁₀ aryl, C₃-C₆ cycloalkyl, 5- or 6-membered heterocyclyl or 5- or 6-membered heteroaryl group,
(b) -L-CR(A)₂ or -L-CH═C(A)₂ wherein R is hydrogen or C₁-C₄ alkyl, L is as defined above and each A is the same or different and is as defined above,
(c) -L^{/}-Het-A^{/}, wherein Het is —O—, —S— or —NR^{/}— wherein R^{/} is hydrogen, C₁-C₄ alkyl, phenyl or —(C₁-C₄ alkyl)-phenyl, A^{/} is -L-A, -L-CR(A)₂ or -L-CH═C(A)₂, L^{/} is a C₁-C₆ alkyl moiety, L is as defined above and each A is the same or different and is as defined above,
(d) -L-CO—NR₃R₄ or -L-CS—NR₃R₄ wherein L is as defined above and either (i) R₃ and R₄, together with the nitrogen atom to which they are attached, form a 5- to 7-membered heterocyclyl group or (ii) R₃ represents hydrogen, C₁-C₄ alkyl, phenyl, —(C₁-C₄ alkyl)-phenyl or —(C₁-C₄ alkyl)-CHPh₂ and R₄ represents C₁-C₄ alkyl, A, —(C₁-C₄ alkyl)-A, —(CH₂)ₘ—CH(A)₂, —CH[(CH₂)ₘA]₂, —(CH₂)ₘ—CO-A, —(CH₂)ₘ—O—CH(A)₂, —(CH₂)ₘ—S—CH(A)₂, —(CH₂)ₘ—S(O)—CH(A)₂, —(CH₂)ₘ—S(O)₂—CH(A)₂, —NH—CO—N(A)₂, —N(A)₂ or -A-O-A, wherein each A is the same or different and is as defined above and m is 0, 1, 2, 3 or 4,
(e) —CO-L-NR₃R₄ or —CS-L-NR₃R₄ wherein L, R₃ and R₄ are as defined above,
(f) —CO-A^{/}, or —CS-A^{/}, wherein A^{/} is as defined above,
(g) -L^{/}-O—N═C(A)₂, —CO-L^{/}-O—N═C(A)₂ wherein L^{/} is as defined above and each A is the same or different and is as defined above, or
(h) -L^{/}-NR—CO—NR₃R₄ or -L^{/}-NR—CS—NR₃R₄ wherein L^{/}, R, R₃ and R₄ are as defined above,
   wherein

said aryl, heteroaryl, carbocyclyl and heterocyclyl groups are optionally fused to one or two cyclic moieties selected from phenyl rings and 5- to 6-membered heterocyclyl and heteroaryl groups, and

said aryl, heteroaryl, carbocyclyl and heterocyclyl groups are unsubstituted or are substituted by 1, 2 or 3 substituents which are the same or different and are selected from halogen, C₁-C₄ alkyl, hydroxy, amino, (C₁-C₄ alkyl)amino, C₁-C₄ alkoxy, C₁-C₄ haloalkyl, C₁-C₄ haloalkoxy, C₁-C₄ alkylthio, C₁-C₄ haloalkylthio, —NH—CO—(C₁-C₂ alkyl), —CO—(C₁-C₂ alkyl), —CO₂—(C₁-C₂ alkyl), 5-membered heteroaryl, phenyl and —CHPh₂ substituents, the phenyl and heteroaryl moieties in said substituents being unsubstituted or substituted by one or two further substituents selected from halogen atoms, C₁-C₂ alkyl groups, C₁-C₂ alkoxy groups and —NH—CO—(C₁-C₂ alkyl) groups,

provided that (a) when R₂ is -L-A, A is monocyclic and (b) when R₂ is —CO-A^{/} or —CS-A^{/}, A is a said C₆-C₁₀ aryl group.

Further preferred compounds of formula (I) are those in which

X is —CH—;

n is from 0 to 3;

p is 0;

m is 1, 2 or 3;

each R₁ is the same or different and is a hydroxy, halogen, C₁-C₄ alkyl, C₁-C₄ haloalkyl, C₁-C₄ alkoxy, C₁-C₄ haloalkoxy, C₁-C₄ alkylthio, or C₁-C₄ haloalkylthio group; and

R₂ is either
(a) -L-A wherein L is a direct bond or a C₁-C₆ alkyl moiety and A is a C₆-C₁₀ aryl, C₃-C₆ cycloalkyl, 5- or 6-membered heterocyclyl or 5- or 6-membered heteroaryl group,
(b) -L-CR(A)₂ or -L-CH═C(A)₂ wherein R is hydrogen or C₁-C₄ alkyl, L is as defined above and each A is the same or different and is as defined above,
(c) -L^{/}-Het-A^{/}, wherein Het is —O—, —S— or —NR^{/}— wherein R^{/} is hydrogen, C₁-C₄ alkyl, phenyl or —(C₁-C₄ alkyl)-phenyl, A^{/} is -L-A, -L-CR(A)₂ or -L-CH═C(A)₂, L^{/} is a C₁-C₆ alkyl moiety, L is as defined above and each A is the same or different and is as defined above,
(d) -L-CO—NR₃R₄ or -L-CS—NR₃R₄ wherein L is as defined above and either (i) R₃ and R₄, together with the nitrogen atom to which they are attached, form a 5- to 7-membered heterocyclyl group or (ii) R₃ represents hydrogen, C₁-C₄ alkyl, —(C₁-C₄ alkyl)-phenyl or —(C₁-C₄ alkyl)-CHPh₂ and R₄ represents C₁-C₄ alkyl, A, —(C₁-C₄ alkyl)-A, —(CH₂)ₘ—CH(A)₂, —CH[(CH₂)ₘA]₂ or —(CH₂)ₘCO-A wherein each A is the same or different and is as defined above and m is 0, 1, 2, 3 or 4,
(e) —CO-L-NR₃R₄ or —CS-L-NR₃R₄ wherein L, R₃ and R₄ are as defined above,
(f) —CO-A^{/} or —CS-A^{/} wherein A^{/} is as defined above, or
(g) -L^{/}-O—N═C(A)₂, —CO-L^{/}-O—N═C(A)₂ wherein L^{/} is as defined above and each A is the same or different and is as defined above,
   wherein

said aryl, heteroaryl, carbocyclyl and heterocyclyl groups are optionally fused to one or two cyclic moieties selected from phenyl rings and 5- to 6-membered heterocyclyl and heteroaryl groups, and

said aryl, heteroaryl, carbocyclyl and heterocyclyl groups are unsubstituted or are substituted by 1, 2 or 3 substituents which are the same or different and are selected from halogen, C₁-C₄ alkyl, hydroxy, C₁-C₄ alkoxy, C₁-C₄ haloalkyl, C₁-C₄ haloalkoxy, C₁-C₄ alkylthio, C₁-C₄ haloalkylthio, phenyl and —CHPh₂ substituents, the phenyl moieties in said substituents being unsubstituted or substituted by one or two halogen atoms,

provided that (a) when R₂ is defined according to option (a), it is a —(C₁-C₄ alkyl)-phenyl group or a —(C₁-C₄ alkyl)-(5- to 6-membered heteroaryl) group, the phenyl and heteroaryl moieties being unsubstituted or substituted by 1 or 2 substituents selected from C₁-C₂ haloalkyl, halogen, C₁-C₂ haloalkylthio, C₁-C₂ haloalkoxy, C₁-C₂ alkyl and phenyl substituents and (b) when R₂ is defined according to option (f) it is —CO—CH₂—CH(R)₂ or —COR^{/}, wherein each R is the same or different and is a phenyl or halophenyl moiety and R^{/} is a benzimidazolyl group.

More preferred compounds of formula (I) are compounds wherein:

X is —N— or —CH—;

n is 0 or 1;

each R₁ is the same or different and is C₁-C₂ alkyl, hydroxy or C₁-C₂ alkoxy;

p is 0 or 1;

R₁^{/} is cyano, —NH—CO—CH₃, —NH—S(O)₂—CH₃, —O—S(O)₂—CH₃, —N[SO₂—CH₃]₂ or —S(O)₂—OH;

m is 1, 2 or 3; and

R₂ is either
(a) -L-A wherein L represents a direct bond or a C₁-C₄ alkyl moiety, for example a methyl, ethyl or propyl moiety, and A is a phenyl, thienyl, triazolyl, pyridyl, fluorenyl, thiazolyl, tetrahydroisoquinolinyl, 9H-carbazolyl, indolinyl, 9H-xanthenyl or benzimidazolyl group, which group is unsubstituted or substituted by one or two substituents selected from halogen, C₁-C₂ alkyl, hydroxy, amino, C₁-C₂ alkoxy, C₁-C₂ haloalkyl, C₁-C₂ haloalkoxy, C₁-C₂ haloalkylthio, —NH—CO—CH₃ and phenyl substituents,
(b) -L-CR(A)₂ or -L-CH═C(A)₂ wherein R is hydrogen or methyl, L is as defined above and each A is the same or different and is as defined above,
(c) -L^{/}-Het-A^{/} wherein Het is —O— or —NR^{/}— wherein R^{/} is hydrogen, C₁-C₄ alkyl or benzyl, A^{/} is -L-A, -L-CR(A)₂ or -L-CH═C(A)₂, L^{/} is a C₁-C₄ alkyl moiety, for example a methyl, ethyl or propyl moiety, L is as defined above, R is as defined above and each A is the same or different and is as defined above,
(d) -L-CO—NR₃R₄ wherein L is as defined above and either (i) R₃ and R₄, together with the nitrogen atom to which they are attached, form a morpholino, thiomorpholino, S-oxo-thiomorpholino, S,S-dioxo-thiomorpholino, pyrrolidinyl, piperazinyl or homopiperidinyl ring which is (a) optionally fused to one or two cyclic moieties selected from phenyl rings and 5- to 6-membered heteroaryl rings, and (b) unsubstituted or substituted by one or two substituents selected from C₁-C₄ alkyl, C₁-C₄ haloalkyl, C₁-C₄ alkoxy, C₁-C₄ alkylthio, halogen, phenyl, —CHPh₂, —CO—(C₁-C₂ alkyl), —CO₂—(C₁-C₂ alkyl) and 5- to 6-membered heteroaryl substituents, the phenyl and heteroaryl moieties in said substituents being unsubstituted or substituted by one or two further substituents selected from halogen atoms, C₁-C₂ alkyl groups, C₁-C₂ alkoxy groups and —NH—CO—(C₁-C₂ alkyl) groups, or (ii) R₃ represents hydrogen, C₁-C₄ alkyl or an unsubstituted benzyl, phenyl, hydroxyphenyl or —(C₁-C₂ alkyl)-CHPh₂ group and R₄ represents C₁-C₄ alkyl, fluorenyl, phenyl, pyridyl, —(C₁-C₄ alkyl)-phenyl, —(C₁-C₄ alkyl)-(5- to 6-membered heteroaryl), —(CH₂)ₘ-(9H-carbazolyl), —(CH₂)ₘ-indolinyl, —(CH₂)ₘ-(9H-xanthenyl), —(CH₂)ₘ—O—CHA^{//}A^{///}, —(CH₂)ₘ—S—CHA^{//}A^{///}, —(CH₂)ₘ—S(O)—CHA^{//}A^{///}, —(CH₂)ₘ—S(O)₂—CHA^{//}A^{///}, —NH—CO—N(Phenyl)₂, —N(Phenyl)₂ or -A^{//}-O-A^{///}, —(CH₂)ₘ—CHA^{//}A^{///}, —CH[(CH₂)ₙPh]₂ or —(CH₂)ₚ—CO—R where m is 0, 1, 2 or 3, A^{//} and A^{///} are the same or different and each represent phenyl or a 5- or 6-membered heteroaryl group, n is 0, 1 or 2, p is 1, 2 or 3 and R is 5- or 6-membered heterocyclic group fused to a phenyl ring, for example a tetrahydroisoquinoline group, the cyclic moieties in said R₄ groups being unsubstituted or substituted by a halogen atom, C₁-C₂ alkyl, hydroxy, amino or C₁-C₂ alkoxy group,
(e) —CO-L-NR₃R₄ or —CS-L-NR₃R₄ wherein L, R₃ and R₄ are as defined above,
(f) —CO-A^{/} or —CS-A^{/} where A^{/} is as defined above,
(g) —CO-L^{/}-O—N═C(A)₂ wherein L^{/} is as defined above and each A is the same or different and is as defined above; or
(h) -L^{/}-NR—CO—NR₃R₄ or -L^{/}-NR—CS—NR₃R₄ wherein L^{/}, R, R₃ and R₄ are as defined above,
   provided that when R₂ is -L-A, A is monocyclic.

Further preferred compounds of formula (I) compounds of formula (1a) 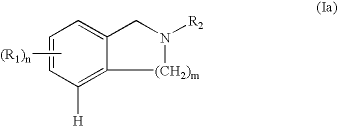
wherein

n is 0 or 1;

each R₁ is the same or different and is C₁-C₂ alkyl, hydroxy or C₁-C₂ alkoxy;

m is 1, 2 or 3; and

R₂ is either
(a) -L-A wherein L represents a direct bond or a C₁-C₄ alkyl moiety, for example a methyl, ethyl or propyl moiety, and A is a phenyl, thienyl, triazolyl, pyridyl, fluorenyl, thiazolyl, tetrahydroisoquinolinyl or benzimidazolyl group, which group is unsubstituted or substituted by one or two substituents selected from halogen, C₁-C₂ alkyl, hydroxy, C₁-C₂ alkoxy, C₁-C₂ haloalkyl, C₁-C₂ haloalkoxy, C₁-C₂ haloalkylthio and phenyl substituents,
(b) -L-CR(A)₂ or -L-CH═C(A)₂ wherein R is hydrogen or methyl, L is as defined above and each A is the same or different and is as defined above,
(c) -L^{/}-Het-A^{/} wherein Het is —O— or —NR^{/}— wherein R^{/} is hydrogen, C₁-C₄ alkyl or benzyl, A^{/} is -L-A, -L-CR(A)₂ or -L-CH═C(A)₂, L^{/} is a C₁-C₄ alkyl moiety, for example a methyl, ethyl or propyl moiety, L is as defined above, R is as defined above and each A is the same or different and is as defined above,
(d) -L-CO—NR₃R₄ wherein L is as defined above and either (i) R₃ and R₄, together with the nitrogen atom to which they are attached, form a morpholino, piperazinyl or homopiperidinyl ring which is (a) substituted or unsubstituted by one or two substituents selected from C₁-C₄ alkyl, C₁-C₄ haloalkyl, halogen, phenyl and —CHPh₂ substituents, the phenyl moieties in said substituents being unsubstituted or substituted by one or two halogen atoms and (b) optionally fused to one or two phenyl rings, or (ii) R₃ represents hydrogen, C₁-C₄ alkyl or an unsubstituted benzyl or —CH₂—CH₂—CHPh₂ group and R₄ represents C₁-C₄ alkyl, fluorenyl, —(C₁-C₄ alkyl)-phenyl, —(C₁-C₄ alkyl)-(5- to 6-membered heteroaryl), —(CH₂)ₘ—CHA^{//}A^{///} where m is 0, 1, 2 or 3 and A^{//} and A^{///} are the same or different and each represent phenyl or a 5- or 6-membered heteroaryl group, —CH[(CH₂)ₙPh]₂, wherein n is 0, 1 or 2, or —(CH₂)ₚ—CO—R wherein p is 1, 2 or 3 and R is 5- or 6-membered heterocyclic group fused to a phenyl ring, for example a tetrahydroisoquinoline group, the cyclic moieties in said R₄ groups being unsubstituted or substituted by a halogen atom, C₁-C₂ alkyl or C₁-C₂ alkoxy group,
(e) —CO-L-NR₃R₄ or —CS-L-NR₃R₄ wherein L, R₃ and R₄ are as defined above,
(f) —CO-A^{/} or CS-A^{/} wherein A^{/} is as defined above, or
(g) —CO-L^{/}-O—N═C(A)₂ wherein L^{/} is as defined above and each A is the same or different and is as defined above,

provided that when R₂ is defined according to option (a) it is a benzyl, —CH₂-thienyl or —CH₂-triazolyl group, the phenyl and heteroaryl moieties being unsubstituted or substituted by 1 or 2 substituent selected from C₁-C₂ haloalkyl, halogen, C₁-C₂ haloalkylthio, C₁-C₂ haloalkoxy, C₁-C₂ alkyl and phenyl substituents.

Examples of these particularly preferred compounds of the invention include:
1. 2-(3,5-bis-trifluoromethyl-benzyl)-1,2,3,4-tetrahydro-isoquinolin-6-ol
2. 2-(2-chloro-6-fluoro-benzyl)-1,2,3,4-tetrahydro-isoquinolin-8-ol
3. 2-(2,5-difluoro-benzyl)-1,2,3,4-tetrahydro-isoquinolin-8-ol
4. 2-(3,5-difluoro-benzyl)-1,2,3,4-tetrahydro-isoquinolin-8-ol
5. 2-(4-trifluoromethylsulfanyl-benzyl)-1,2,3,4-tetrahydro-isoquinolin-8-ol
6. 2-(3,5-bis-trifluoromethyl-benzyl)-1,2,3,4-tetrahydro-isoquinolin-8-ol
7. 2-(2-dibenzylamino-ethyl)-1,2,3,4-tetrahydro-isoquinolin-8-ol
8. 2-[4,4-bis-(4-fluoro-phenyl)-butyl]-1,2,3,4-tetrahydro-isoquinolin-8-ol
9. 2-[4,4-bis-(4-hydroxy-3,5-dimethyl-phenyl)-pentyl]-1,2,3,4-tetrahydro-isoquinolin-8-ol
10. 2-(8-hydroxy-3,4-dihydro-1H-isoquinolin-2-yl)-1-(3-phenyl-2,3-dihydro-benzo[1,4]oxazin-4-yl)-ethanone
11. 2-(2-benzyloxy-propyl)-1,2,3,4-tetrahydro-isoquinolin-8-ol
12. 2-(2,2-diphenyl-ethyl)-1,2,3,4-tetrahydro-isoquinolin-8-ol
13. N-benzhydryl-2-(3,4-dihydro-1H-isoquinolin-2-yl)-acetamide
14. 2-(3,4-dihydro-1H-isoquinolin-2-yl)-N-(9H-fluoren-9-yl)-acetamide
15. N-(1-benzyl-2-phenyl-ethyl)-2-(3,4-dihydro-1H-isoquinolin-2-yl)-acetamide
16. 2-(3,4-dihydro-1H-isoquinolin-2-yl)-N-(1,2-diphenyl-ethyl)-acetamide
17. 2-(3,4-dihydro-1H-isoquinolin-2-yl)-N-(3,3-diphenyl-propyl)-acetamide
18. N-benzhydryl-2-(8-methoxy-3,4-dihydro-1H-isoquinolin-2-yl)-acetamide
19. N-(9H-fluoren-9-yl)-2-(8-methoxy-3,4-dihydro-1H-isoquinolin-2-yl)-acetamide
20. N-benzyl-2-(8-methoxy-3,4-dihydro-1H-isoquinolin-2-yl)-N-phenyl-acetamide
21. N-(3,3-diphenyl-propyl)-2-(8-methoxy-3,4-dihydro-1H-isoquinolin-2-yl)-acetamide
22. N,N-dibenzyl-2-(8-hydroxy-3,4-dihydro-1H-isoquinolin-2-yl)-acetamide
23. 2-thiophen-2-ylmethyl-1,2,3,4-tetrahydro-isoquinolin-8-ol
24. N-benzhydryl-2-(8-hydroxy-3,4-dihydro-1H-isoquinolin-2-yl)-acetamide
25. N-benzyl-2-(8-hydroxy-3,4-dihydro-1H-isoquinolin-2-yl)-N-phenyl-acetamide
26. N-(9H-fluoren-9-yl)-2-(8-hydroxy-3,4-dihydro-1H-isoquinolin-2-yl)-acetamide
27. N-(3,3-diphenyl-propyl)-2-(8-hydroxy-3,4-dihydro-1H-isoquinolin-2-yl)-acetamide
28. 2-(5-phenyl-2H-[1,2,3]triazol-4-ylmethyl)-1,2,3,4-tetrahydro-isoquinolin-8-ol
29. 1-(3,4-dihydro-1H-isoquinolin-2-yl)-2-(2,2-diphenyl-ethylamino)-ethanone
30. 1-(3,4-dihydro-1H-isoquinolin-2-yl)-2-(3,3-diphenyl-propylamino)-ethanone
31. 1-(3,4-dihydro-1H-isoquinolin-2-yl)-2-[[2-(3,4-dihydro-1H-isoquinolin-2-yl)-2-oxo-ethyl]-(3,3-diphenyl-propyl)-amino]-ethanone
32. 2-dibenzylamino-1-(3,4-dihydro-1H-isoquinolin-2-yl)-ethanone
33. N-(3,3-diphenyl-propyl)-2-(7-methoxy-3,4-dihydro-1H-isoquinolin-2-yl)-acetamide
34. N,N-dibenzyl-2-(7-methoxy-3,4-dihydro-1H-isoquinolin-2-yl)-acetamide dibenzyl-[2-(7-methoxy-3,4-dihydro-1H-isoquinolin-2-yl)-ethyl]-amine
35. 2-(2,2-diphenyl-ethyl)-1,2,3,4-tetrahydro-isoquinoline
36. 2-(2,2-diphenyl-ethyl)-8-methoxy-1,2,3,4-tetrahydro-isoquinoline
37. 2-[4,4-bis-(4-fluoro-phenyl)-butyl]-1,2,3,4-tetrahydro-isoquinoline
38. 2-[4,4-bis-(4-fluoro-phenyl)-butyl]-8-methoxy-1,2,3,4-tetrahydro-isoquinoline
39. 1-(3,4-dihydro-1H-isoquinolin-2-yl)-3,3-bis-(4-fluoro-phenyl)-propan-1-one
40. 2-(3,4-dihydro-1H-isoquinolin-2-yl)-N-[1-(5-methyl-thiazol-2-yl)-ethyl]-acetamide
41. 2-(8-hydroxy-3,4-dihydro-1H-isoquinolin-2-yl)-N-[1-(5-methyl-thiazol-2-yl)-ethyl]-acetamide
42. (3,3-diphenyl-propyl)-[2-(7-methoxy-3,4-dihydro-1H-isoquinolin-2-yl)-ethyl]-amine
43. 2-(benzhydryl-amino)-1-(3,4-dihydro-1H-isoquinolin-2-yl)-ethanone
44. dibenzyl-[2-(3,4-dihydro-1H-isoquinolin-2-yl)-ethyl]-amine
45. [2-(3,4-dihydro-1H-isoquinolin-2-yl)-ethyl]-(3,3-diphenyl-propyl)-amine
46. 2-{(2,2-diphenyl-ethyl)-[2-(8-methoxy-3,4-dihydro-1H-isoquinolin-2-yl)-2-oxo-ethyl]-amino}-1-(8-methoxy-3,4-dihydro-1H-isoquinolin-2-yl)-ethanone
47. 2-{benzhydryl-[2-(8-methoxy-3,4-dihydro-1H-isoquinolin-2-yl)-2-oxo-ethyl]-amino}-1-(8-methoxy-3,4-dihydro-1H-isoquinolin-2-yl)-ethanone
48. 2-(benzhydryl-amino)-1-(8-methoxy-3,4-dihydro-1H-isoquinolin-2-yl)-ethanone
49. 2-(2,2-diphenyl-ethylamino)-1-(8-methoxy-3,4-dihydro-1H-isoquinolin-2-yl)-ethanone
50. (1H-benzoimidazol-5-yl)-(3,4-dihydro-1H-isoquinolin-2-yl)-methanone
51. N-(2,2-diphenyl-ethyl)-2-(8-methoxy-3,4-dihydro-1H-isoquinolin-2-yl)-acetamide
52. 1-(4-benzhydryl-piperazin-1-yl)-2-(8-methoxy-3,4-dihydro-1H-isoquinolin-2-yl)-ethanone
53. 1-{4-[bis-(4-fluoro-phenyl)-methyl]-piperazin-1-yl}-2-(8-methoxy-3,4-dihydro-1H-isoquinolin-2-yl)-ethanone
54. 1-(4-benzhydryl-piperazin-1-yl)-2-(3,4-dihydro-1H-isoquinolin-2-yl)-ethanone
55. 1-{4-[bis-(4-fluoro-phenyl)-methyl]-piperazin-1-yl}-2-(3,4-dihydro-1H-isoquinolin-2-yl)-ethanone
56. 2-(1,3-dihydro-isoindol-2-yl)-N-(2,2-diphenyl-ethyl)-acetamide
57. 1-(4-benzhydryl-piperazin-1-yl)-2-(1,3-dihydro-isoindol-2-yl)-ethanone
58. 1-{4-[bis-(4-fluoro-phenyl)-methyl]-piperazin-1-yl}-2-(1,3-dihydro-isoindol-2-yl)-ethanone
59. 2-benzhydrylideneaminooxy-1-(3,4-dihydro-1H-isoquinolin-2-yl)-ethanone
60. 2-(3,4-dihydro-1H-isoquinolin-2-yl)-N-(2,2-diphenyl-ethyl)-acetamide
61. 2-(1,3-dihydro-isoindol-2-yl)-N-(3,3-diphenyl-propyl)-acetamide
62. N-(3,3-diphenyl-propyl)-3-(8-methoxy-3,4-dihydro-1H-isoquinolin-2-yl)-propionamide
63. 2-(8-methoxy-3,4-dihydro-1H-isoquinolin-2-yl)-N-(phenyl-pyridin-2-yl-methyl)-acetamide
64. 3,4-dihydro-1H-isoquinoline-2-carbothioic acid (2,2-diphenyl-ethyl)-amide
65. N-benzhydryl-2-(1,3-dihydro-isoindol-2-yl)-acetamide
66. 3,4-dihydro-1H-isoquinoline-2-carbothioic acid benzhydryl-amide
67. 8-methoxy-3,4-dihydro-1H-isoquinoline-2-carbothioic acid benzhydryl-amide
68. 8-methoxy-3,4-dihydro-1H-isoquinoline-2-carbothioic acid (2,2-diphenyl-ethyl)-amide
69. 2-benzhydrylideneaminooxy-1-(8-methoxy-3,4-dihydro-1H-isoquinolin-2-yl)-ethanone
70. 2-(di-pyridin-2-yl-methyleneaminooxy)-1-(8-methoxy-3,4-dihydro-1H-isoquinolin-2-yl)-ethanone
71. 2-(8-methoxy-3,4-dihydro-1H-isoquinolin-2-yl)-1-(3-phenyl-2,3-dihydro-benzo[1,4]oxazin-4-yl)-ethanone
72. 2-(8-methoxy-3,4-dihydro-1H-isoquinolin-2-yl)-1-phenoxazin-10-yl-ethanone
73. 1-(10,11-dihydro-dibenzo[b,f]azepin-5-yl)-2-(8-methoxy-3,4-dihydro-1H-isoquinolin-2-yl)-ethanone
74. 2-[3-(2,2-diphenyl-vinyloxy)-propyl]-8-methoxy-1,2,3,4-tetrahydro-isoquinoline
75. 4-methoxy-1,3-dihydro-isoindole-2-carbothioic acid benzhydryl-amide
76. 7-methoxy-1,3,4,5-tetrahydro-benzo[c]azepine-2-carbothioic acid benzhydryl-amide
77. 7-methoxy-1,3,4,5-tetrahydro-benzo[c]azepine-2-carbothioic acid (2,2-diphenyl-ethyl)-amide
78. N,N-diisopropyl-2-(8-methoxy-3,4-dihydro-1H-isoquinolin-2-yl)-acetamide
79. N,N-dibenzyl-2-(8-methoxy-3,4-dihydro-1H-isoquinolin-2-yl)-acetamide
80. N-benzhydryl-2-(4-methoxy-1,3-dihydro-isoindol-2-yl)-acetamide
81. N-(4,4-diphenyl-butyl)-2-(8-methoxy-3,4-dihydro-1H-isoquinolin-2-yl)-acetamide
82. N-(4,4-diphenyl-butyl)-2-(4-methoxy-1,3-dihydro-isoindol-2-yl)-acetamide
83. N-benzhydryl-2-(7-methoxy-1,3,4,5-tetrahydro-benzo[c]azepin-2-yl)-acetamide
84. N-(2,2-diphenyl-ethyl)-2-(7-methoxy-1,3,4,5-tetrahydro-benzo[c]azepin-2-yl)-acetamide
85. N-(3,3-diphenyl-propyl)-2-(7-methoxy-1,3,4,5-tetrahydro-benzo[c]azepin-2-yl)-acetamide
86. N,N-dibenzyl-2-(7-methoxy-1,3,4,5-tetrahydro-benzo[c]azepin-2-yl)-acetamide
87. N,N-dibenzyl-3-(8-methoxy-3,4-dihydro-1H-isoquinolin-2-yl)-propionamide
88. N-(3,3-diphenyl-propyl)-2-(4-methoxy-1,3-dihydro-isoindol-2-yl)-acetamide
89. N-(2,2-diphenyl-ethyl)-2-(4-methoxy-1,3-dihydro-isoindol-2-yl)-acetamide
90. 2-(1,3-Dihydro-isoindol-2-yl)-N-(2,2-diphenyl-ethyl)-acetamide
91. N,N-Dibenzyl-2-(8-ethoxy-3,4-dihydro-1H-isoquinolin-2-yl)-acetamide
92. N-(4,4-Diphenyl-butyl)-2-(8-ethoxy-3,4-dihydro-1H-isoquinolin-2-yl)-acetamide
93. 2-(8-Ethoxy-3,4-dihydro-1H-isoquinolin-2-yl)-1-(3-phenyl-2,3-dihydro-benzo[1,4]oxazin-4-yl)-ethanone
94. N-(3-Benzhydryloxy-propyl)-2-(8-methoxy-3,4-dihydro-1H-isoquinolin-2-yl)-acetamide
95. 2-(1,3-Dihydro-isoindol-2-yl)-N-(3,3-diphenyl-propyl)-acetamide
96. N-(2-Benzhydrylsulfanyl-ethyl)-2-(8-methoxy-3,4-dihydro-1H-isoquinolin-2-yl)-acetamide
97. 2-(8-Allyloxy-3,4-dihydro-1H-isoquinolin-2-yl)-N-(3,3-diphenyl-propyl)-acetamide
98. 2-(4-Amino-1,3-dihydro-isoindol-2-yl)-N-(2,2-diphenyl-ethyl)-acetamide
99. 2-(4-Amino-1,3-dihydro-isoindol-2-yl)-N-(3,3-diphenyl-propyl)-acetamide
100. 2-(4-Amino-1,3-dihydro-isoindol-2-yl)-N-(4,4-diphenyl-butyl)-acetamide
101. 2-(4-Amino-1,3-dihydro-isoindol-2-yl)-N,N-dibenzyl-acetamide
102. 2-[4,4-Bis-(4-fluoro-phenyl)-butyl]-2,3-dihydro-1H-isoindol-4ylamine
103. N-[2-(Diphenyl-methanesulfinyl)-ethyl]-2-(8-methoxy-3,4-dihydro-1H-isoquinolin-2-yl)-acetamide
104. N-[2-(Diphenyl-methanesulfonyl)-ethyl]-2-(8-methoxy-3,4-dihydro-1H-isoquinolin-2-yl)-acetamide
105. 2-(8-Methoxy-3,4-dihydro-1H-isoquinolin-2-yl)-N-(1-phenyl-ethyl)-acetamide
106. 2-(3,4-Dihydro-1H-isoquinolin-2-yl)-N-(1-phenyl-ethyl)-acetamide
107. 2-(Benzhydryl-amino)-1-(1,3-dihydro-isoindol-2-yl)-ethanone
108. 2-(8-Amino-3,4-dihydro-1H-isoquinolin-2-yl)-N-benzhydryl-acetamide
109. 2-(8-Amino-3,4-dihydro-1H-isoquinolin-2-yl)-N-(4,4-diphenyl-butyl)-acetamide
110. 2-[4,4-Bis-(4-fluoro-phenyl)-butyl]-1,2,3,4-tetrahydro-isoquinolin-8-ylamine
111. 2-(8-Amino-3,4-dihydro-1H-isoquinolin-2-yl)-N-(2,2-diphenyl-ethyl)-acetamide
112. 2-(8-Acetylamino-3,4-dihydro-1H-isoquinolin-2-yl)-N-(4,4-diphenyl-butyl)-acetamide
113. N-[3,3-Bis-(4-methoxy-phenyl)-propyl]-2-(1,3-dihydro-isoindol-2-yl)-acetamide
114. N-[3,3-Bis-(4-methoxy-phenyl)-propyl]-2-(3,4-dihydro-1H-isoquinolin-2-yl)-acetamide
115. N-[3,3-Bis-(4-methoxy-phenyl)-propyl]-2-(8-methoxy-3,4-dihydro-1H-isoquinolin-2-yl)-acetamide
116. N-[3-(3,4-Bis-acetylamino-phenyl)-3-phenyl-propyl]-2-(3,4-dihydro-1H-isoquinolin-2-yl)-acetamide
117. N-(4,4-Diphenyl-butyl)-2-(8-methanesulfonylamino-3,4-dihydro-1H-isoquinolin-2-yl)-acetamide
118. N-[Bis-(4-fluoro-phenyl)-methyl]-2-(1,3-dihydro-isoindol-2-yl)-acetamide
119. N-[Bis-(4-fluoro-phenyl)-methyl]-2-(3,4-dihydro-1H-isoquinolin-2-yl)-acetamide
120. N-[Bis-(4-fluoro-phenyl)-methyl]-2-(8-methoxy-3,4-dihydro-1H-isoquinolin-2-yl)-acetamide
121. N-[Bis-(4-fluoro-phenyl)-methyl]-2-(6,7-dimethoxy-3,4-dihydro-1H-isoquinolin-2-yl)-acetamide
122. 3-(5-Amino-3,4-dihydro-1H-isoquinolin-2-yl)-N-(3,3-diphenyl-propyl)-propionamide
123. 2-(5-Amino-3,4-dihydro-1H-isoquinolin-2-yl)-N-(2,2-diphenyl-ethyl)-acetamide
124. 2-(Dimethoxy-3,4-dihydro-1H-isoquinolin-2-yl)-N-(4,4-diphenyl-butyl)-acetamide
125. 3-(6,7-Dimethoxy-3,4-dihydro-1H-isoquinolin-2-yl)-N-(3,3-diphenyl-propyl)-propionamide
126. 2-(6,7-Dimethoxy-3,4-dihydro-1H-isoquinolin-2-yl)-N-(2,2-diphenyl-ethyl)-acetamide
127. 3-(8-Amino-3,4-dihydro-1H-isoquinolin-2-yl)-N-(3,3-diphenyl-propyl)-propionamide
128. N-(3-Carbazol-9-yl-propyl)-2-(8-methoxy-3,4-dihydro-1H-isoquinolin-2-yl)-acetamide
129. N-(3-Carbazol-9-yl-propyl)-2-(8-hydroxy-3,4-dihydro-1H-isoquinolin-2-yl)-acetamide
130. N-[3-(5-Chloro-2-methyl-indol-1-yl)-propyl]-2-(8-methoxy-3,4-dihydro-1H-isoquinolin-2-yl)-acetamide
131. N-[3-(5-Chloro-2-methyl-indol-1-yl)-propyl]-2-(8-hydroxy-3,4-dihydro-1H-isoquinolin-2-yl)-acetamide
132. 2-(8-Methoxy-3,4-dihydro-1H-isoquinolin-2-yl)-1-phenoxazin-10-yl-ethanone
133. 1-Benzhydryl-3-[2-(8-methoxy-3,4-dihydro-1H-isoquinolin-2-yl)-ethyl]-thiourea
134. 1-Benzhydryl-3-[2-(6,7-dimethoxy-3,4-dihydro-1H-isoquinolin-2-yl)-ethyl]-thiourea
135. 1-Benzhydryl-3-[2-(8-methoxy-3,4-dihydro-1H-isoquinolin-2-yl)-ethyl]-urea
136. 1-Benzhydryl-3-[2-(6,7-dimethoxy-3,4-dihydro-1H-isoquinolin-2-yl)-ethyl]-urea
137. 1-(2,2-Diphenyl-ethyl)-3-[2-(8-methoxy-3,4-dihydro-1H-isoquinolin-2-yl)-ethyl]-thiourea
138. 1-[2-(6,7-Dimethoxy-3,4-dihydro-1H-isoquinolin-2-yl)-ethyl]-3-(2,2-diphenyl-ethyl)-thiourea
139. 2-(8-Hydroxy-3,4-dihydro-1H-isoquinolin-2-yl)-1-phenothiazin-10-yl-ethanone
140. 2-(8-Methoxy-3,4-dihydro-1H-isoquinolin-2-yl)-1-phenothiazin-10-yl-ethanone
141. 1-(2-Chloro-phenothiazin-10-yl)-2-(8-hydroxy-3,4-dihydro-1H-isoquinolin-2-yl)-ethanone
142. 1-(2-Chloro-phenothiazin-10-yl)-2-(8-methoxy-3,4-dihydro-1H-isoquinolin-2-yl)-ethanone
143. 2-(8-Methoxy-3,4-dihydro-1H-isoquinolin-2-yl)-1-(5-oxo-5H-5lambda*4*-phenothiazin-10-yl)-ethanone
144. 2-(8-Hydroxy-3,4-dihydro-1H-isoquinolin-2-yl)-1-phenoxazin-10-yl-ethanone
145. 2-(8-Hydroxy-3,4-dihydro-1H-isoquinolin-2-yl)-1-(2-trifluoromethyl-phenothiazin-10-yl)-ethanone
146. 2-(8-Methoxy-3,4-dihydro-1H-isoquinolin-2-yl)-1-(2-trifluoromethyl-phenothiazin-10-yl)-ethanone
147. 1-(2-Acetyl-phenothiazin-10-yl)-2-(8-hydroxy-3,4-dihydro-1H-isoquinolin-2-yl)-ethanone
148. 1-(2-Acetyl-phenothiazin-10-yl)-2-(8-methoxy-3,4-dihydro-1H-isoquinolin-2-yl)-ethanone
149. 2-(8-Hydroxy-3,4-dihydro-1H-isoquinolin-2-yl)-N,N-diphenyl-acetamide
150. 2-(8-Methoxy-3,4-dihydro-1H-isoquinolin-2-yl)-N,N-diphenyl-acetamide
151. 2-(6,7-Dimethoxy-3,4-dihydro-1H-isoquinolin-2-yl)-1-(5,5-dioxo-5H-5lambda*6*-phenothiazin-10-yl)-ethanone
152. 2-(6,7-Dimethoxy-3,4-dihydro-1H-isoquinolin-2-yl)-1-phenothiazin-10-yl-ethanone
153. 1-(2-Chloro-phenothiazin-10-yl)-2-(6,7-dimethoxy-3,4-dihydro-1H-isoquinolin-2-yl)-ethanone
154. 2-(6,7-Dimethoxy-3,4-dihydro-1H-isoquinolin-2-yl)-1-(2-trifluoromethyl-phenothiazin-10-yl)-ethanone
155. 1-(2-Acetyl-phenothiazin-10-yl)-2-(6,7-dimethoxy-3,4-dihydro-1H-isoquinolin-2-yl)-ethanone
156. 2-(6,7-Dimethoxy-3,4-dihydro-1H-isoquinolin-2-yl)-1-(2-methylsulfanyl-phenothiazin-10-yl)-ethanone
157. 2-(6,7-Dimethoxy-3,4-dihydro-1H-isoquinolin-2-yl)-1-(5-oxo-5H-5lambda*4*-phenothiazin-10-yl)-ethanone
158. 2-(8-Hydroxy-3,4-dihydro-1H-isoquinolin-2-yl)-1-(2-methylsulfanyl-phenothiazin-10-yl)-ethanone
159. 2-(8-Methoxy-3,4-dihydro-1H-isoquinolin-2-yl)-1-(2-methylsulfanyl-phenothiazin-10-yl)-ethanone
160. Phenothiazine-10-carboxylic acid [2-(8-hydroxy-3,4-dihydro-1H-isoquinolin-2-yl)-ethyl]-amide
161. Phenothiazine-10-carboxylic acid [2-(6,7-dimethoxy-3,4-dihydro-1H-isoquinolin-2-yl)-ethyl]-amide
162. Phenothiazine-10-carboxylic acid [2-(8-methoxy-3,4-dihydro-1H-isoquinolin-2-yl)-ethyl]-amide
163. Phenoxazine-10-carboxylic acid [2-(8-hydroxy-3,4-dihydro-1H-isoquinolin-2-yl)-ethyl]-amide
164. Phenoxazine-10-carboxylic acid [2-(8-methoxy-3,4-dihydro-1H-isoquinolin-2-yl)-ethyl]-amide
165. Phenoxazine-10-carboxylic acid [2-(6,7-dimethoxy-3,4-dihydro-1H-isoquinolin-2-yl)-ethyl]-amide
166. N-[3,3-Bis-(4-fluoro-phenyl)-propyl]-3-(8-methoxy-3,4-dihydro-1H-isoquinolin-2-yl)-propionamide
167. (8-Hydroxy-3,4-dihydro-1H-isoquinolin-2-yl)-acetic acid N′,N′-diphenyl-hydrazide
168. (8-Methoxy-3,4-dihydro-1H-isoquinolin-2-yl)-acetic acid N′,N′-diphenyl-hydrazide
169. (6,7-Dimethoxy-3,4-dihydro-1H-isoquinolin-2-yl)-acetic acid N′,N′-diphenyl-hydrazide
170. 4-[2-(8-Hydroxy-3,4-dihydro-1H-isoquinolin-2-yl)-acetyl]-3,4-dihydro-2H-benzo[1,4]oxazine-2-carboxylic acid ethyl ester
171. 4-[2-(8-Methoxy-3,4-dihydro-1H-isoquinolin-2-yl)-acetyl]-3,4-dihydro-2H-benzo[1,4]oxazine-2-carboxylic acid ethyl ester
172. 2-(8-Methoxy-3,4-dihydro-1H-isoquinolin-2-yl)-N-(4-phenoxy-phenyl)-acetamide
173. 2-(5,8-Dihydro-6H-[1,7]naphthyridin-7-yl)-1-phenoxazin-10-yl-ethanone
174. 1-[2-(8-Methoxy-3,4-dihydro-1H-isoquinolin-2-yl)-ethyl]-3-(4-phenoxy-phenyl)-urea
175. 2-(8-Amino-3,4-dihydro-1H-isoquinolin-2-yl)-1-phenoxazin-10-yl-ethanone
176. 2-(8-Hydroxy-3,4-dihydro-1H-isoquinolin-2-yl)-N-(4-hydroxy-phenyl)-N-phenyl-acetamide
177. N-(4-Hydroxy-phenyl)-2-(8-methoxy-3,4-dihydro-1H-isoquinolin-2-yl)-N-phenyl-acetamide
178. 2-(1,3-Dihydro-isoindol-2-yl)-1-phenoxazin-10-yl-ethanone
179. 2-(8-Hydroxy-3,4-dihydro-1H-isoquinolin-2-yl)-N-(9H-xanthen-9-yl)-acetamide
180. 2-(8-Methoxy-3,4-dihydro-1H-isoquinolin-2-yl)-N-(9H-xanthen-9-yl)-acetamide
181. 2-(5,8-Dihydro-6H-[1,7]naphthyridin-7-yl)-N,N-diphenyl-acetamide
182. 2-(8-Methoxy-3,4-dihydro-1H-isoquinolin-2-yl)-N,N-bis-(4-methoxy-phenyl)-acetamide
183. 2-(8-Hydroxy-3,4-dihydro-1H-isoquinolin-2-yl)-N,N-bis-(4-methoxy-phenyl)-acetamide
184. 2-(8-Methoxy-3,4-dihydro-1H-isoquinolin-2-yl)-N-(2-phenoxy-phenyl)-acetamide
185. 2-(8-Hydroxy-3,4-dihydro-1H-isoquinolin-2-yl)-N-(2-phenoxy-phenyl)-acetamide
186. 1-[(8-Methoxy-3,4-dihydro-1H-isoquinolin-2-yl)-acetyl]-4,4-diphenylsemicarbazide
187. 2-(8-Methoxy-3,4-dihydro-1H-isoquinolin-2-yl)-1-[2-(5-methyl-[1,3,4]oxadiazol-2-yl)-2,3-dihydro-benzo[1,4]oxazin-4-yl]-ethanone
188. N-(3-Amino-pyridin-2-yl)-N-(2-hydroxy-phenyl)-2-(8-methoxy-3,4-dihydro-1H-isoquinolin-2-yl)-acetamide
189. 3-(8-Methoxy-3,4-dihydro-1H-isoquinolin-2-yl)-1-phenoxazin-10-yl-propan-1-one
190. 3-(8-Hydroxy-3,4-dihydro-1H-isoquinolin-2-yl)-1-phenoxazin-10-yl-propan-1-one
191. Methanesulfonic acid 2-(2-oxo-2-phenoxazin-10-yl-ethyl)-1,2,3,4-tetrahydro-isoquinolin-8-yl ester
192. 1-(2,3-Dihydro-benzo[1,4]oxazin-4-yl)-2-(8-methoxy-3,4-dihydro-1H-isoquinolin-2-yl)-ethanone
193. 2-(7-Hydroxy-3,4-dihydro-1H-isoquinolin-2-yl)-1-phenoxazin-10-yl-ethanone
194. 2-(6-Hydroxy-3,4-dihydro-1H-isoquinolin-2-yl)-1-phenoxazin-10-yl-ethanone
195. 2-(5-Hydroxy-3,4-dihydro-1H-isoquinolin-2-yl)-1-phenoxazin-10-yl-ethanone
196. 2-(4-Methoxy-1,3-dihydro-isoindol-2-yl)-1-phenoxazin-10-yl-ethanone
197. N-Methanesulfonyl-N-[2-(2-oxo-2-phenoxazin-10-yl-ethyl)-1,2,3,4-tetrahydro-isoquinolin-8-yl]-methanesulfonamide
198. N-[2-(2-Oxo-2-phenoxazin-10-yl-ethyl)-1,2,3,4-tetrahydro-isoquinolin-8-yl]-methanesulfonamide
199. 2-(8-Methoxy-3,4-dihydro-1H-isoquinolin-2-yl)-1-(1-methyl-1H-4-oxa-1,2,9-triaza-cyclopenta[b]naphthalen-9-yl)-ethanone
200. 2-(8-Methoxy-3,4-dihydro-1H-isoquinolin-2-yl)-1-phenoxazin-10-yl-propan-1-one
201. Phenoxazine-10-carboxylic acid [2-(5,8-dihydro-6H-[1,7]naphthyridin-7-yl)-ethyl]-amide
202. 2-(4-Hydroxy-1,3-dihydro-isoindol-2-yl)-1-phenoxazin-10-yl-ethanone
203. Methanesulfonic acid 2-(2-oxo-2-phenoxazin-10-yl-ethyl)-2,3-dihydro-1H-isoindol-4-yl ester
204. 1-Carbazol-9-yl-2-(8-methoxy-3,4-dihydro-1H-isoquinolin-2-yl)-ethanone
205. 2-(8-Methoxy-3,4-dihydro-1H-isoquinolin-2-yl)-1-(3-methyl-2,3-dihydro-benzo[1,4]oxazin-4-yl)-ethanone
206. 1-(3-tert-Butyl-2,3-dihydro-benzo[1,4]oxazin-4-yl)-2-(8-methoxy-3,4-dihydro-1H-isoquinolin-2-yl)-ethanone
207. 1-(11H-Dibenzo[b,f][1,4]oxazepin-10-yl)-2-(8-methoxy-3,4-dihydro-1H-isoquinolin-2-yl)-ethanone
208. 1-(3-Ethyl-2,3-dihydro-benzo[1,4]oxazin-4-yl)-2-(8-methoxy-3,4-dihydro-1H-isoquinolin-2-yl)-ethanone
209. 2-(2-Oxo-2-phenoxazin-10-yl-ethyl)-1,2,3,4-tetrahydro-isoquinoline-8-sulfonic acid
210. N-[2-(2-Oxo-2-phenoxazin-10-yl-ethyl)-2,3-dihydro-1H-isoindol-4-yl]-methanesulfonamide
211. 1-(3-tert-Butyl-2,3-dihydro-benzo[1,4]oxazin-4-yl)-2-(8-hydroxy-3,4-dihydro-1H-isoquinolin-2-yl)-ethanone
212. 2-(8-Methoxy-3,4-dihydro-1H-isoquinolin-2-yl)-1-[3-(4-methoxy-phenyl)-2,3-dihydro-benzo[1,4]oxazin-4-yl]-ethanone
213. 1-[3-(2,5-Dimethoxy-phenyl)-2,3-dihydro-benzo[1,4]oxazin-4-yl]-2-(8-methoxy-3,4-dihydro-1H-isoquinolin-2-yl)-ethanone
214. N-(4-{4-[2-(8-Methoxy-3,4-dihydro-1H-isoquinolin-2-yl)-acetyl]-3,4-dihydro-2H-benzo[1,4]oxazin-3-yl}-phenyl)-acetamide
215. 1-[3-(4-Fluoro-phenyl)-2,3-dihydro-benzo[1,4]oxazin-4-yl]-2-(8-methoxy-3,4-dihydro-1H-isoquinolin-2-yl)-ethanone
216. 1-[3-(3,4-Dimethoxy-phenyl)-2,3-dihydro-benzo[1,4]oxazin-4-yl]-2-(8-methoxy-3,4-dihydro-1H-isoquinolin-2-yl)-ethanone
217. 2-(4-Methoxy-1,3-dihydro-isoindol-2-yl)-1-phenoxazin-10-yl-propan-1-one
218. 1-(1,3-Dihydro-isoindol-2-yl)-2-phenoxazin-10-yl-ethanone
219. 2-(4-Chloro-1,3-dihydro-isoindol-2-yl)-1-phenoxazin-10-yl-ethanone
220. 2-(2-Oxo-2-phenoxazin-10-yl-ethyl)-2,3-dihydro-1H-isoindole-4-carbonitrile

and pharmaceutically acceptable salts thereof.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aralkyl amines or heterocyclic amines.

The compounds of the invention can contain one or more chiral centres. For the avoidance of doubt, the chemical structures depicted herein are intended to embrace all stereoisomers of the compounds shown, including racemic and non-racemic mixtures and pure enantiomers and/or diastereoisomers.

Preferred compounds of the invention are optically active isomers. Thus, for example, preferred compounds of formula (I) containing only one chiral centre include an R enantiomer in substantially pure form, an S enanitiomer in substantially pure form and enantiomeric mixtures which contain an excess of the R enantiomer or an excess of the S enantiomer.

The compounds of formula (I) may be prepared by conventional routes, for example those set out in any of schemes 1 to 10 shown below.

Compounds of formula (1) in which m is 2 and X, R₁, n and R₂ are defined as above (reaction scheme 1) may be prepared from compounds of formula (2) and compounds of formula (3) where X is a leaving group, typically chlorine, using standard methods such as reaction in the presence of a base, for example potassium carbonate. Typically the reaction is performed in a solvent such as methanol, tetrahydrofuran or acetonitrile at a temperature of 95° C. Compounds of formula (2) may be prepared from compounds of formula (4) by standard methods familiar to those skilled in the art such as reduction in the presence of platinum oxide. Alternatively, compounds of formula (2) may be prepared from compounds of formula (5) and formaldehyde by standard methods such as the Pictet-Spengler cyclisation.

Compounds of formula (4) are known compounds or may be prepared by standard methods such as cyclisation of compounds of formula (6) according to the published procedure (Bioorg. Med. Chem. 7 (1999) 2647-2666).

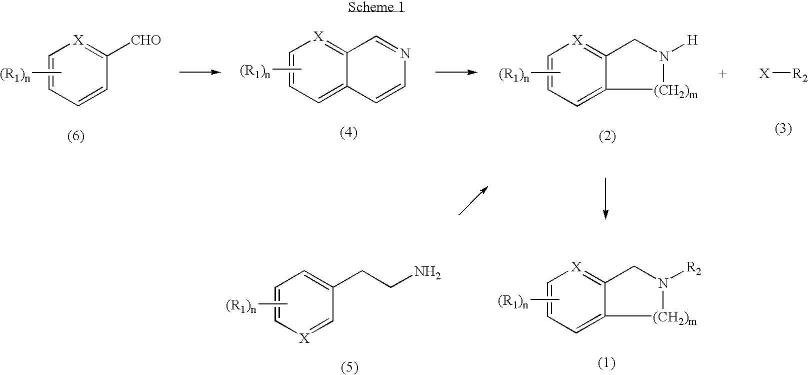

Compounds of formula (1) in which m is 1 and X, R₁, n and R₂ are defined as above (reaction scheme 2) may be prepared from compounds of formula (2) and compounds of formula (3) where X is a leaving group, typically chlorine, using standard methods such as reaction in the presence of a base for example potassium carbonate. Typically the reaction is performed in a solvent such as methanol, tetrahydrofuran or acetonitrile at a temperature of 95° C.

Compounds of formula (2) may be prepared from compounds of formula (7) where X is a leaving group, preferably bromine, by standard methods familiar to those skilled in the art such as alkylation in the presence of an amine. Alternatively, compounds of formula (2) can be prepared from compounds of formula (7) where X is OH converted into a better leaving group such as a mesylate under standard alkylating conditions familiar to those skilled in the art. Compounds of formula (7) may be prepared from dimethylaryl compounds (8) by bromination using a brominating reagent, for example N-bromosuccinimide. Alcohols (9) may be prepared from acids (10) by standard methods such as reduction in the presence of lithium aluminium hydride.

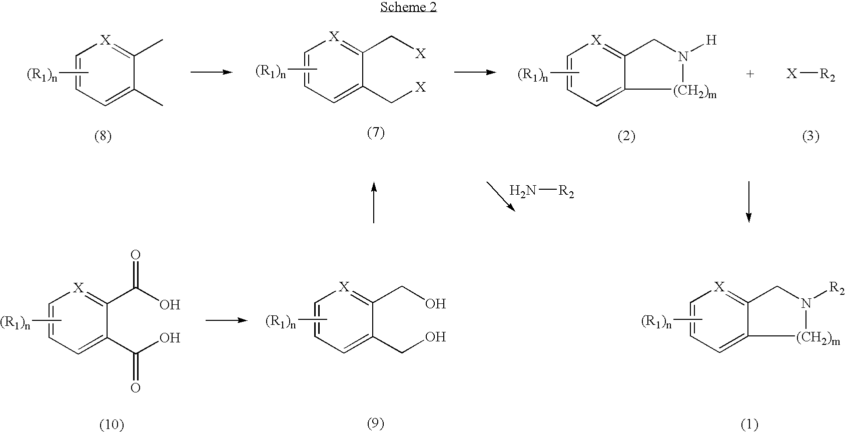

Compounds of formula (I) in which m is 3 and X, R₁, n and R₂ are defined as above (reaction scheme 3) may be prepared from compounds of formula (2) and compounds of formula (3) where X is a leaving group, typically chlorine, using standard methods such as reaction in the presence of a base for example potassium carbonate. Typically the reaction is performed in a solvent such as methanol, tetrahydrofuran or acetonitrile at a temperature of 95° C.

Compounds of formula (2) where m is 3 may be prepared from compounds of formula (11) by reduction in the presence of a metal hydride for example lithium aluminium hydride. Compounds of formula (11) may be prepared from tetralones (12) by standard methods familiar to those skilled in the art such as the Schmidt reaction. Alternatively, compounds of formula (11) may be prepared from tetralones (12) by standard methods familiar to those skilled in the art such as the Beckmann rearrangement or further methods as outlined e.g. in Alicyclic Chemistry, (Martin Grossel, Oxford University Press). Tetralones (12) are either known compounds or can be prepared by analogy with known methods.

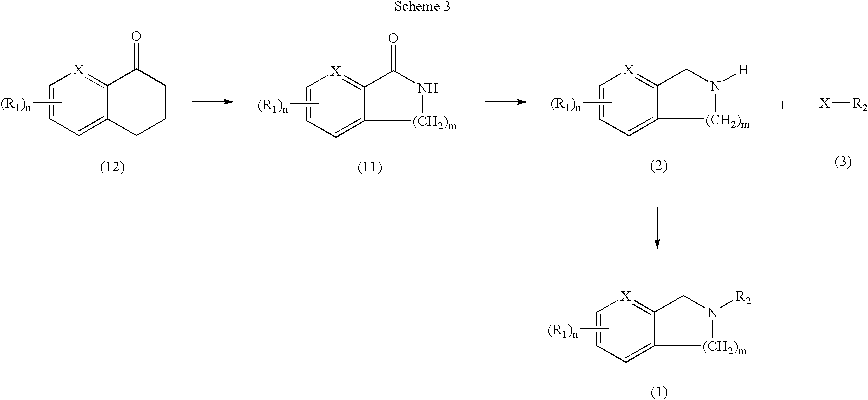

When R₂ is -L-A and L is other than a direct bond, or when R₂ is -L-CR(A)₂, the reaction between the compounds of formulae (2) and (3) in schemes 1, 2 and 3 is typically performed in a solvent such as methanol, tetrahydrofuran or acetonitrile at a temperature of 80° C. When R₂ is -L-A and L is a direct bond, the reaction between, the compounds of formulae (2) and (3) is typically effected by Buchwald coupling. Thus, X in the formula (3) is typically bromine or iodine.

The compounds of formula (3) are known compounds, or may be prepared by known methods. For example, compounds of formula (3) in which R₂ is —(CH₂)₂—CH(A)₂ can be prepared by the reduction of compounds of formula (14) in the presence of a reducing agent such as lithium aluminium hydride followed by halogenation in the presence of a halogenating agent such as PBr₃ (reaction scheme 4). Compounds of formula (14) may be prepared from diarylethenylacids (15) by reduction in the presence of a reducing agent such as palladium. Diarylethenylacids may be prepared from ketones (16) by standard methods familiar to those skilled in the art such as Wittig reaction.

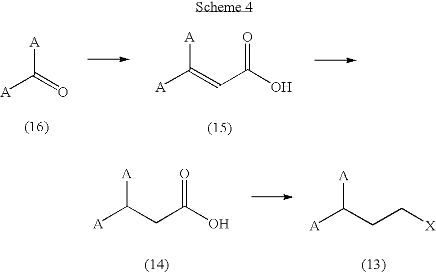

Compounds of formula (3) in which R₂ is -L-CH═C(A)₂ where L and A are defined as above (reaction scheme 5) may be prepared from corresponding carboxylic acids by reduction in the presence of a reducing agent, for example lithium aluminium hydride, followed by halogenation in the presence of a halogenating reagent for example PBr₃.

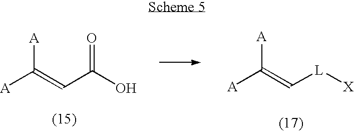

Compounds of formula (3) wherein R₂ is -L^{/}-Het-A^{/} can, for example, be prepared from compounds of formula (19) where Y is a leaving group, by reaction with compounds of formula (20) (reaction scheme 6). Compounds of formula (18) in which A^{/} is —CH₂(A)₂ may also be prepared from compounds of formula (16) and compounds of formula (20) by standard methods familiar to those skilled in the art. Thus, when Het is O or S, compounds (16) and (20) can be condensed in the presence of an acid catalyst, for example PTSA. When Het is NH the reaction between compounds (16) and (20) can be effected by standard methods such as reductive amination in the presence of a reducing agent, for example sodium borohydride.

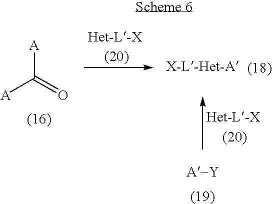

When R₂ is -L-CO—NR₃R₄ the reaction between the compounds of formulae (2) and (3) in schemes 1 to 3 is typically effected in the presence of a base for example triethylamine. Typically the reaction is performed in a solvent such as methanol, tetrahydrofuran or acetonitrile at a temperature of 80° C. Further, compounds of formula (1) wherein R₂ is -L-CS—NR₃R₄ may be prepared from compounds of formula (1) where R₂ is -L-CO—NR₃R₄ by standard methods familiar to those skilled in the art such as sulphonation in the presence of Lawesson's reagent.

Compounds of formula (3) in which R₂ is -L-CO—NR₃R₄ can be prepared from amines (22) and compounds of formula (23), in which X^{/} is Cl or OH, under standard amide coupling reaction conditions (reaction scheme 7). Typically, where X^{/} is Cl, the reaction is effected in the presence of triethylamine.

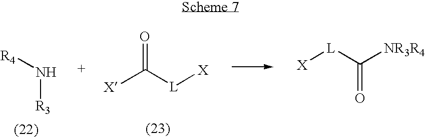

A further method for preparing compounds of formula (1) wherein X, m, R₁ and n are defined as above and R₂ is —CO-L-NR₃R₄ involves the reaction of amides (24) and amines (22) where X is a leaving group, preferably chlorine, using standard methods such as reaction in the presence of a base for example triethylamine (reaction scheme 8). Typically the reaction is performed in a solvent such as methanol, tetrahydrofuran or acetonitrile at a temperature of 80° C. Amides (24) may be prepared from amines (2) and acids (23), wherein X^{/} is Cl or OH, under standard amide coupling reaction conditions. Typically, where X^{/} is Cl, the reaction is effected in the presence of triethylamine.

Alternatively, compounds of formula (1) where R₂ is —CO-L-NR₃R₄, L is a direct bond and R₄ is hydrogen may be prepared from amines (2) by standard methods familiar to those skilled in the art such as alkylation with isocyanates (25). Similarly, compounds of formula (1) where R₂ is —CS-L-NR₃R₄ and L is a direct bond may be prepared from amines (2) by standard methods such as alkylation with isothiocyanates (26). Compounds of formula (1) wherein R₂ is —CS-L-NR₃R₄ can, of course, be prepared from compounds of formula (1) where R₂ is -L-CO—NR₃R₄ by standard methods familiar to those skilled in the art such as sulphonation using Lawesson's reagent.

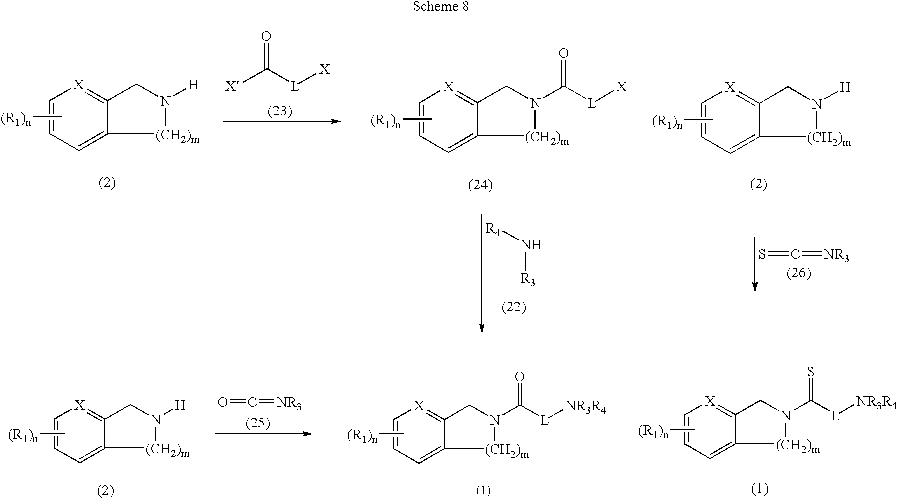

When R₂ is —CO-A^{/}, the reaction between the compounds of formulae (2) and (3) in schemes 1, 2 and 3 is typically effected in the presence of a coupling agent such as EDC/HOBT, HATU or HBTU. Compounds of formula (I) wherein R₂ is —CS-A^{/} can, of course, be prepared from compounds of formula (1) where R₂ is —CO-A^{/} by standard methods familiar to those skilled in the art such as reaction with Lawesson's reagent.

Compounds of formula (3), wherein R₂ is —CO-L^{/}-O—N═C(A)₂ or -L^{/}-O—N═C(A)₂ may be prepared from ketones (16) and hydroxylamine by standard methods familiar to those skilled in the art (reaction scheme 9). In reaction scheme 9, X and X^{/} represent leaving groups, for example chlorine.

Further, an additional method of preparing compounds of formula (I) in which R₂ is —CO-L^{/}-O—N═C(A)₂ or -L^{/}-O—N═C(A)₂ involves the reaction of a compound of formula (31) or (31a), wherein X is a leaving group, typically chlorine, and oximes (29) by standard methods as previously described. Compounds of formulae (31) and (31a) may be prepared from amines (2) and compounds of formulae (30) or (30a) under standard amide coupling conditions as previously described.

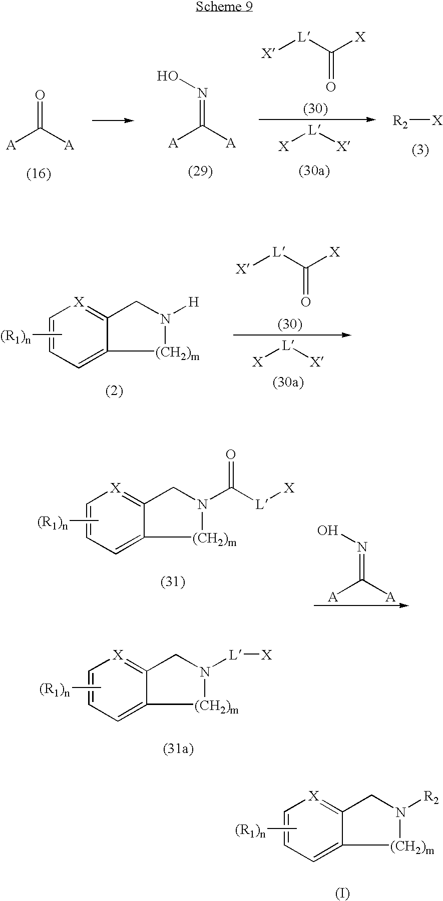

The compounds of the invention are found to be inhibitors of sensory neurone specific sodium channels. The compounds of the invention are therefore therapeutically useful. Accordingly, the preset invention provides a compound of the formula (I), as defined above, or a pharmaceutically acceptable salt thereof, for use in the treatment of the human or animal body. Also provided is a pharmaceutical composition comprising a compound of the formula (I), as defined above, or a pharmaceutically acceptable salt thereof, and a pharmaceutically acceptable carrier or diluent. Said pharmaceutical composition typically contains up to 85 wt % of a compound of the invention. More typically, it contains up to 50 wt % of a compound of the invention. Preferred pharmaceutical compositions are sterile and pyrogen free. Further, the pharmaceutical compositions provided by the invention typically contain a compound of the invention which is a substantially pure optical isomer.

The compounds of the invention may be administered in a variety of dosage forms. Thus, they can be administered orally, for example as tablets, troches, lozenges, aqueous or oily suspensions, dispersible powders or granules. Preferred pharmaceutical compositions of the invention are compositions suitable for oral administration, for example tablets and capsules.

Compositions suitable for oral administration may, if required, contain a colouring or flavoring agent. Typically, a said capsule or tablet comprises from 5 to 500 mg, preferably 10 to 500 mg, more preferably 15 to 100 mg, of a compound of formula (I) or a pharmaceutically acceptable salt thereof.

The compounds of the invention may also be administered parenterally, whether subcutaneously, intravenously, intramuscularly, intrasternally, transdermally or by infusion techniques. The compounds may also be administered as suppositories.

One preferred route of administration is inhalation. The major advantages of inhaled medications are their direct delivery to the area of rich blood supply in comparison to many medications taken by oral route. Thus, the absorption is very rapid as the alveoli have an enormous surface area and rich blood supply and first pass metabolism is bypassed.

Preferred pharmaceutical compositions of the invention therefore include those suitable for inhalation. The present invention also provides an inhalation device containing such a pharmaceutical composition. Typically said device is a metered dose inhaler (MDI), which contains a pharmaceutically acceptable chemical propellant to push the medication out of the inhaler. Typically, said propellant is a fluorocarbon.

Further preferred inhalation devices include nebulizers. Nebulizers are devices capable of delivering fine liquid mists of medication through a “mask” that fits over the nose and mouth, using air or oxygen under pressure. They are frequently used to treat those with asthma who cannot use an inhaler, including infants, young children and acutely ill patients of all ages.

Said inhalation device can also be, for example, a rotary inhaler or a dry powder inhaler, capable of delivering a compound of the invention without a propellant.

Typically, said inhalation device contains a spacer. A spacer is a device which enables individuals to inhale a greater amount of medication directly into the lower airways, where it is intended to go, rather than into the throat. Many spacers fit on the end of an inhaler; for some, the canister of medication fits into the device. Spacers with withholding chambers and one-way valves prevent medication from escaping into the air. Many people, especially young children and the elderly, may have difficulties coordinating their inhalation with the action necessary to trigger a puff from a metered dose inhaler. For these patients, use of a spacer is particularly recommended.

Another preferred route of administration is intranasal administration. The nasal cavity's highly permeable tissue is very receptive to medication and absorbs it quickly and efficiently, more so than drugs in tablet form. Nasal drug delivery is less painful and invasive than injections, generating less anxiety among patients. Drugs can be delivered nasally in smaller doses than medication delivered in tablet form. By this method absorption is very rapid and first pass metabolism is bypassed, thus reducing inter-patient variability. Nasal delivery devices further allow medication to be administered in precise, metered doses. Thus, the pharmaceutical compositions of the invention are typically suitable for intranasal administration. Further, the present invention also provides an intranasal device containing such a pharmaceutical composition.

A further preferred route of administration is transdermal administration. The present invention therefore also provides a transdermal patch containing a compound of the invention, or a pharmaceutically acceptable salt thereof. Also preferred is sublingual administration. The present invention therefore also provides a sub-lingual tablet comprising a compound of the invention or a pharmaceutically acceptable salt thereof.

A compound of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

The compounds of the present invention are therapeutically useful in the treatment or prophylaxis of conditions involving sodium ion flux through a sensory neurone specific (SNS) channel of a sensory neurone. Said condition may be one of hypersensitivity for example resulting from a concentration of SNS channels at the site of nerve injury or in axons following nerve injury, or may be sensitisation of the neurone for example at sites of inflammation as a result of inflammatory mediators.

Said compounds of the invention are therefore most preferred for their use in the treatment or prophylaxis of any condition involving hypersensitivity or sensitisation of a sensory neurone specific (SNS) channel of a sensory neurone.

Accordingly, the present invention also provides the use of a compound of the formula (I), or a pharmaceutically acceptable salt thereof, in the manufacture of a medicament for use in the treatment or prophylaxis of a condition involving sodium ion flux through a sensory neurone specific (SNS) channel of a sensory neurone, more specifically hypersensitivity of a sensory neurone or sensitisation of a sensory neurone specific (SNS) channel of a sensory neurone. Also provided is a method of treating a patient suffering from or susceptible to a condition involving sodium ion flux through a sensory neurone specific (SNS) channel of a sensory neurone, more specifically hypersensitivity of a sensory neurone or sensitisation of a sensory neurone specific (SNS) channel of a sensory neurone, which method comprises administering to said patient an effective amount of a compound of formula (I), of a pharmaceutically acceptable salt thereof.

The term treatment in this context is deemed to cover any effect from a cure of said condition to alleviation of any or all of the symptoms. The compounds of the invention may, where appropriate, be used prophylactically to reduce the incidence or severity of said conditions.

Specific conditions in which SNS channels are present and believed to be involved include pain, for example chronic and acute pain, hypersensitivity disorders such as bladder dysfunction and bowel disorders which may or may not also have associated pain, and demyelinating diseases.

SNS sodium channels are known to mediate pain transmission. Typically, the compounds of the invention are therefore used as analgesic agents. SNS specific sodium channels have been identified as being particularly important in the transmission of pain signals. The compounds of the invention are accordingly particularly effective in alleviating pain. Typically, therefore, said medicament is for use in alleviating pain and said patient is suffering from or susceptible to pain. The compounds of the invention are effective in alleviating both chronic and acute pain.

Acute pain is generally understood to be a constellation of unpleasant sensory, perceptual and emotional experiences of certain associate autonomic (reflex) responses, and of psychological and behavioural reactions provoked by injury or disease. A discussion of acute pain can be found at Halpern (1984) Advances in Pain Research and Therapy, Vol. 7, p. 147. Tissue injury provokes a series of noxious stimuli which are transduced by nociceptors to impulses transmitted to the spinal cord and then to the upper part of the nervous system. Examples of acute pains which can be alleviated with the compounds of the invention include musculoskeletal pain, for example joint pain, lower back pain and neck pain, dental pain, post-operative pain, obstetric pain, for example labour pain, acute headache, neuralgia, myalgia, and visceral pain.

Chronic pain is generally understood to be pain that persists beyond the usual course of an acute disease or beyond a reasonable time for an injury to heal. A discussion of chronic pain can be found in the Halpern reference given above. Chronic pain is sometimes a result of persistent dysfunction of the nociceptive pain system. Examples of chronic pains which can be alleviated with the compounds of the invention include trigeminal neuralgia, post-herpetic neuralgia (a form of chronic pain accompanied by skin changes in a dermatomal distribution following damage by acute Herpes Zoster disease), diabetic neuropathy, causalgia, “phantom limb” pain, pain associated with osteoarthritis, pain associated with rheumatoid arthritis, pain associated with cancer, pain associated with HIV, neuropathic pain, migraine and other conditions associated with chronic cephalic pain, primary and secondary hyperalgesia, inflammatory pain, nociceptive pain, tabes dorsalis, spinal cord injury pain; central pain, post-herpetic pain, noncardiac chest pain, irritable bowel syndrome and pain associated with bowel disorders and dyspepsia.

Some of the chronic pains set out above, for example, trigeminal neuralgia, diabetic neuropathic pain, causalgia, phantom limb pain and central post-stroke pain, have also been classified as neurogenic pain. One non-limiting definition of neurogenic pain is pain caused by dysfunction of the peripheral or central nervous system in the absence of nociceptor stimulation by trauma or disease. The compounds of the invention can, of course, be used to alleviate or reduce the incidence of neurogenic pain

Examples of bowel disorders which can be treated or prevented with the compounds of the invention include inflammatory bowel syndrome and inflammatory bowel disease, for example Crohn's disease and ulcerative colitis.

Examples of bladder dysfunctions which can be treated or prevented with the compounds of the invention include bladder hyper reflexia and bladder inflammation, for example interstitial cystitis, overactive (or unstable) bladder (OAB), more specifically urinary incontinence, urgency, frequency, urge incontinence and nocturia. The compounds of the invention can also bemused to alleviate pain associated with bladder hyper reflexia or bladder inflammation.

Examples of demyelinating diseases which can be treated or prevented with the compounds of the invention are those in which SNS channels are known to be expressed by the demyelinated neurones and which may or may not also have associated pain. A specific example of such a demyelinating disease is multiple sclerosis. The compounds of the invention can also be used to alleviate pain associated with demyelinating diseases such as multiple sclerosis.

The compounds of the invention have additional properties as they are capable of inhibiting voltage dependent sodium channels. They can therefore be used, for example, to protect cells against damage or disorders which results from overstimulation of sodium channels.

The compounds of the invention are useful in the treatment and prevention of peripheral and central nervous system disorders. They can therefore additionally be used in the treatment or prevention of an affective disorder, an anxiety disorder, a behavioural disorder, a cardiovascular disorder, a central or peripheral nervous system degenerative disorder, a central nervous system injury, a cerebral ischaemia, a chemical injury or substance abuse disorder, a cognitive disorder, an eating disorder, an eye disease, Parkinson's disease or a seizure disorder.

Examples of affective disorders which can be treated or prevented with the compounds of the invention include mood disorders, bipolar disorders (both Type 1 and Type II) such as seasonal affective disorder, depression, manic depression, atypical depression and monodepressive disease, schizophrenia, psychotic disorders, mania and paranoia.

Examples of anxiety disorders which can be treated or prevented with the compounds of the invention include generalised anxiety disorder (GAD), panic disorder, panic disorder with agoraphobia, simple (specific) phobias (e.g. arachnophobia, performance anxiety such as public speaking), social phobias, post-traumatic stress disorder, anxiety associated with depression, and obsessive compulsive disorder (OCD).

Examples of behavioural disorders which can be treated or prevented with the compounds of the invention include behavioural and psychological signs and symptoms of dementia, age-related behavioural disorders, pervasive development disorders such as autism, Asperger's Syndrome, Retts syndrome and disintegrative disorder, attention deficit disorder, aggressivity, impulse control disorders and personality disorder.

Examples of cardiovascular disorders which can be treated or prevented with the compounds of the invention include cardiac arrthymia, atherosclerosis, cardiac arrest, thrombosis, complications arising from coronary artery bypass surgery, myocardial infarction, reperfusion injury, intermittant claudication, ischaemic retinopathy, angina, pre-eclampsia, hypertension, congestive cardiac failure, restenosis following angioplasty, sepsis and septic shock.

Examples of central and peripheral nervous system degenerative disorders which can be treated or prevented with the compounds of the invention include corticobasal degeneration, disseminated sclerosis, Freidrich's ataxia; motorneurone diseases such as amyotrophic lateral sclerosis and progressive bulbar atrophy, multiple system atrophy, myelopathy, radiculopathy, peripheral neuropathies such as diabetic neuropathy, tabes dorsalis, drug-induced neuropathy and vitamin deficiency, systemic lupus erythamatosis, granulomatous disease, olivo-ponto-cerebellar atrophy, progressive pallidal atrophy, progressive supranuclear palsy and spasticity.

Examples of central nervous system injuries which can be treated with the compounds of the invention include traumatic brain injury, neurosurgery (surgical trauma), neuroprotection for head injuries, raised intracranial pressure, cerebral oedema, hydrocephalus and spinal cord injury.

Examples of cerebral ischaemias which can be treated or prevented with the compounds of the invention include transient ischaemic attack, stroke, for example thrombotic stroke, ischaemic stroke, embolic stroke, haemorrhagic stroke or lacunar stroke, subarachnoid haemorrhage, cerebral vasospasm, peri-natal asphyxia, drowning, cardiac arrest and subdural haematoma.

Examples of chemical injuries and substance abuse disorders which can be treated or prevented with the compounds of the invention include drug dependence, for example opiate dependence, benzodiazepine addition, amphetamine addiction and cocaine addiction, alcohol dependence, methanol toxicity, carbon monoxide poisoning and butane inhalation.

Examples of cognitive disorders which can be treated or prevented with the compounds of the invention include dementia, Alzheimer Disease, Frontotemporal dementia, multi-infarct dementia, AIDS dementia, dementia associated with Huntingtons Disease, Lewy body Dementia, Senile dementia, age-related memory impairment, cognitive impairment associated with dementia, Korsakoff syndrome and dementia pugilans.

Examples of eating disorders which can be treated or prevented with the compounds of the invention include anorexia nervosa, bulimia, Prader-Willi syndrome and obesity.

Examples of eye diseases which can be treated or prevented with the compounds of the invention include drug-induced optic neuritis, cataract, diabetic neuropathy, ischaemic retinopathy, retinal haemorrhage, retinitis pigmentosa, acute glaucoma, in particular acute normal tension glaucoma, chronic glaucoma, in particular chronic normal tension glaucoma; macular degeneration, retinal artery occlusion and retinitis.

Examples of Parkinson's diseases which can be treated or prevented with the compounds of the invention include drug-induced Parkinsonism, post-encephalitic Parkinsonism, Parkinsonism induced by poisoning (for example MPTP, manganese or carbon monoxide poisoning), Dopa-responsive dystonia-Parkinsonism, posttraumatic Parkinson's disease (punch-drunk syndrome), Parkinson's with on-off syndrome, Parkinson's with freezing (end of dose deterioration) and Parkinson's with prominent dyskinesias.

Examples of seizure disorders which can be treated or prevented with the compounds of the invention include epilepsy and post-traumatic epilepsy, partial epilepsy (simple partial seizures, complex partial seizures, and partial seizures secondarily generalised seizures), generalised seizures, including generalised tonicclonic seizures (grand mal), absence seizures (petit mal), myoclonic seizures, atonic seizures, clonic seizures, and tonic seizures, Lennox Gastaut, West Syndrome (infantile spasms), multiresistant seizures and seizure prophylaxis (antiepileptogenic).

The compounds of the present invention are also useful in the treatment and prevention of tinnitus.

A therapeutically effective amount of a compound of the invention is administered to a patient. A typical dose is from about 0.001 to 50 mg per kg of body weight, for example 0.01 to 10 mg, according to the activity of the specific compound, the age, weight and conditions of the subject to be treated, the type and severity of the disease and the frequency and route of administration. Preferably, daily dosage levels are from 5 mg to 2 g.

The following Examples illustrate the invention. They do not, however, limit the invention in any way. In this regard, it is important to understand that the particular assays used in the Examples section are designed only to provide an indication of activity in inhibiting SNS specific sodium channels. A negative result in any one particular assay is not determinative.

### EXAMPLES

The HPLC analysis of Examples 1 to 8, 14 to 29, 32 to 35, 40 to 44 and 98 to 223 was conducted in the following manner: Solvent: MeCN/H₂O/0.05% NH₃, 5-95% gradient H₂O-6 min; Column: Phenomenex 50×4.6 mm i.d., C18 reverse phase; and Flow rate: 1.5 mL/min, unless indicated otherwise.

The HPLC analysis of Examples 9 to 13, 30, 31, 36 to 39 and 45 to 48 was conducted in the following manner: Solvent: MeCN/H₂O/0.05% NH₃, 5-95% gradient H₂O-10 min; Column: Phenomenex 50×4.6 mm i.d., C18 reverse phase; and Flow rate: 1.5 mL/min, unless indicated otherwise.

The HPLC analysis of Examples 49 to 56, 58, 59 and 61 to 97 was conducted in the following manner: Solvent: MeCN/H₂O/0.05% NH₃, 5-95% gradient H₂O-6 min; Column: Xterra 50×4.60 i.d., C18 reverse phase; and Flow rate: 1.5 mL/min, unless indicated otherwise.

The HPLC analysis of Example 60 was conducted in the following manner: Solvent: MeCN/H₂O/0.05% NH₃, 5-95%-gradient H₂O-10 min; Column: Xterra 50×4.60 i.d., C18 reverse phase; and Flow rate: 1.5 mL/min.

### Example 1

### N-Benzhydryl-2-chloro-acetamide

To a stirred solution of aminodiphenylmethane (Aldrich A5,360-5) (4.36 g, 25.3 mmol) in CH₂Cl₂ (50 mL) was added Et₃N (Aldrich 47,128-3) (2.81 g, 27.77 mmol). The reaction mixture was cooled to approximately 10° C. and chloroacetylchloride (Aldrich 10,449-3) (3.14 g, 27.83 mmol) was added drop-wise over 5 min. The reaction mixture was stirred for 2 h and quenched by the addition of distilled H₂O (50 mL). The layers were separated and the organic layer washed with brine (50 mL), dried (Na₂SO₄) and the solvent removed in vacuo. The residue was purified by flash column chromatography to afford the title compound as a white solid (0.78 g, 12%): HPLC retention time 3.67 min. Mass Spectrum (ES+) m/z 260 (M+H).

The following compounds were synthesized from the appropriate diphenylalkylamine and chloroacetylchloride according to the method described above:
2-Chloro-N-(2,2-diphenyl-ethyl)-acetamide;
2-Chloro-N-(3,3-diphenyl-propyl)-acetamide;
N-Benzyl-2-chloro-N-phenyl-acetamide;
N,N-Dibenzyl-2-chloro-acetamide;
2-Chloro-N-(9H-flurenyl-9-yl)-acetamide;
N,N-Dibenzyl-3-chloro-propionamide;
2-Chloro-1-(3,4-dihydro-1H-isoquinolin-2-yl)-ethanone; and
2-Chloro-1-(8-methoxy-3,4-dihydro-1H-isoquinolin-2-yl)-ethanone.

### Example 2

### 2-Chloro-N-(4,4-diphenyl-butyl)-acetamide

To a stirred solution of 1-Bromo-3,3-diphenylpropane (Acros 2719123.1) (2 g, 7.27 mmol) in dimethyl sulphoxide (5 mL) was added potassium cyanide (Aldrich 20,781-0) (0.57 g, 8.73 mmol). The reaction mixture was stirred at room temperature for 19 h and quenched by the addition of distilled H₂O (20 mL). The resulting solution was extracted with EtOAc (3×20 mL) the combined organic layers dried (Na₂SO₄), filtered and the solvent removed in vacuo. The resulting residue was dissolved in anhydrous tetrahydrofuran (25 mL) and borane-tetrahydrofuran complex (Aldrich 17,619-2) (1M, 27 mL, 27 mmol) was added drop wise over 5 min. The reaction mixture was heated at reflux for 2 h, cooled to 0° C. and quenched with CH₃OH (10 mL). The solvent was removed in vacuo and the residue azeotroped with CH₃OH (3×15 mL). The residue was dissolved in CH₂Cl₂ (20 mL) and Et₃N (1.39 g, 13.69 mmol) was added. The reaction mixture was cooled to approximately 10° C. and chloroacetylchloride (Aldrich 10,449-3) (1.55 g, 12.44 mmol) was added drop-wise over 5 min. The reaction mixture was stirred for 4 h and quenched with distilled H₂O (20 mL). The organic layer was separated, dried (MgSO₄) and the solvent removed in vacuo. The residue was purified by flash column chromatography to afford the title compound as a viscous oil (1.8 g, 85%): HPLC retention time 4.04 min. Mass Spectrum (ES+) m/z 302 (M+H).

### Example 3

### 3-Chloro-N-(3,3-diphenyl-propyl)-propionamide

To a stirred solution of 3,3 Diphenylpropylamine (Acros 15948-0250) (6.5 g, 30.7 mmol) in CH₂Cl₂ (50 mL) was added Et₃N (Aldrich 47,128-3) (2.81 g, 27.77 mmol). The reaction mixture was cooled to approximately 10° C. and 3-chloropropionyl chloride (Aldrich C6,912-8) (4.29 g, 30.7 mmol) was added drop-wise over 5 min. The reaction mixture was stirred for 2 h and quenched by the addition of distilled H₂O (50 mL). The layers were separated and the organic layer washed with brine (50 mL), dried (Na₂SO₄) and the solvent remove in vacuo. The residue was purified by flash column chromatography and recrystallisation from EtOAc to afford the title compound as a white solid (3.1 g, 33%): HPLC retention time 3.98 min. Mass Spectrum (ES+) m/z 302 (M+H).

### Example 4

### 8-Methoxyisoquinoline

Ref: Y. Yoshida et al Bioorg. Med. Chem. 7 (1999) 2647-2666.

To a 1 L round bottom flask, equipped with a Dean-Stark trap, was added 2-methoxybenzaldehyde (Aldrich 10,962-2) (23.8 g, 175 mmol) in benzene (850 mL). To this stirred solution was added 2,2-dimethoxyethylamine (Aldrich 12,196-7) (18.3 g, 175 mmol). The reaction mixture was refluxed for 5 h, cooled to room temperature and the solvent removed in vacuo. The residue was dissolved in tetrahydrofuran (238 mL) and cooled to c.a. −10° C., (external temperature maintained between −8° C. to −10° C. with acetone/card-ice). To this cooled solution was added ethyl chloroformate (Aldrich 18,589-2) (18.9 g, 174 mmol) over c.a., 5 min. The reaction mixture was allowed to warm to room temperature and treated with trimethyl phosphite (Aldrich T7,970-7) (25 mL, 212 mmol). The reaction mixture was stirred at room temperature for 60 h, and evaporated in vacuo to give an oil. This oil was dissolved in CH₂Cl₂ (238 mL) and cooled to 0° c. (external temperature), treated with titanium tetrachloride (Aldrich 20,856-6) (200 g, 1.0 mol) over c.a. 8 min, warmed to room temperature, heated at reflux for 3 h, cooled to room temperature and stirred overnight. The reaction mixture was diluted with CH₂Cl₂ (800 mL) and cooled to c.a. 0° C. and basified with 30% sodium hydroxide solution. The neutralised mixture was filtered through celite/sand diluting with c.a. 5 L of CH₂Cl₂. The CH₂Cl₂ layer was separated and dried over MgSO₄, filtered and the solvent removed in vacuo. The resulting brown oil is purified by flash column chromatography using CH₂Cl₂/CH₃OH, 90/10, v/v as mobile phase to give the title compound as a red oil (19.7 g, 70%). ¹H NMR (400 MHz, DMSO-d₆) δ 4.02 (3H), 7.12 (1H), 7.55 (1H), 7.75 (1H), 7.8 (1H), 8.50 (1H), 9.55 (1H).

### Example 5

### Isoquinolin-8-ol

Ref: Y. Yoshida et al Bioorg. Med. Chem. 7 (1999) 2647-2666.

To a stirred solution of 8-methoxyisoquinoline (7.0 g, 44 mmol) in anhydrous CH₂Cl₂ (60 mL) cooled in an ice bath, was added over 0.5 h, boron tribromide, 1M in CH₂Cl₂ (Aldrich 21,122-2) (219 mL, 219 mmol). The reaction mixture was warmed to room temperature, heated at reflux for 2 h cooled to −78° C., and decomposed by the addition of CH₃OH (150 mL). The reaction mixture was warmed to room temperature, heated at reflux for 0.5 h and the solvent removed in vacuo. The residue was azeotroped with CH₃OH (3×100 mL) and suspended in H₂O (150 mL). To this suspension was added CH₂Cl₂ (300 mL) and with vigorous stirring neutralised to c.a. 7.0 with ammonia (0.88). The CH₂Cl₂ layer was separated and the aqueous layer extracted with CH₂Cl₂ (2×200 mL). The combined layers were dried (Na₂SO₄) and the solvent removed in vacuo. The residue was purified by flash column chromatography to give the title compound as a pale yellow solid. (6.87 g, 98%). ¹H NMR (400 MHz, DMSO-d₆) δ 7.10 (1H), 7.45 (1H), 7.65 (1H), 7.75(1H), 8.50 (1H), 9.50 (1H), 10.90 (1H).

### Example 6

### 1,2,3,4-Tetrahydro-isoquinolin-8-ol acetate salt

U.S. Pat. No. 3,575,983

To a stirred solution of Isoquinolin-8-ol (2.0 g, 13.8 mmol) in ethanol (120 mL) was added acetic acid (2 mL) and platinum (IV) oxide (Aldrich 45,992-5) (0.2 g). The reaction mixture was hydrogenated at ca. 4 bar for 18 h. The catalyst was filtered off and the solvent removed in vacuo to give the title compound as a tan solid (5.2 g, 92%): HPLC retention time 2.0 min. Mass Spectrum (ES+) m/z 150 (M+H).

### Example 7

### 8-Methoxy-1,2,3,4-tetrahydro-isoquinoline acetate salt

Prepared according to the method described in Example 6: HPLC retention time 3.33 min. Mass Spectrum (ES+) m/z 164 (M+H).

### Example 8

### 2-(2-Dibenzylamino-ethyl)1,2,3,4-tetrahydro-isoquinolin-8-ol)

To a stirred suspension of 1,2,3,4-Tetrahydro-isoquinolin-8-ol acetate salt (11.0 g, 4.78 mmol) in MeCN (50 mL) was added N-(chloroethy)dibenzylamine hydrochloride (Aldrich 29,136-6) (1.42 g, 4.78 mmol), tetrabutylammonium iodide (Aldrich 14,077-5) (0.29 g, 0.79 mmol) and potassium carbonate (Acros) (0.66 g, 4.78 mmol). The reaction mixture was heated at 95° C. for 7 h and cooled to room temperature, filtered and the solvent removed in vacuo. The residue was dissolved in CH₂Cl₂ (80 mL), washed with H₂O (25 mL), dried (Na₂SO₄) and the solvent remove in vacuo. The residue was purified by flash column chromatography using CH₂Cl₂/CH₃OH/ammonia, 95/5/0.2, v/v/v, as mobile phase to give the title compound as a low melting solid (0.71 g, 39%): 1H NMR (400 MHz, CDCl3) δ_{H} 2.6-2.9 (8H), 3.55 (2H), 3.65(4H), 6.5(1H), 6.95(1H), 7.2-7.5(11H). HPLC retention time 7.27 min. Mass Spectrum (ES+) m/z 373 (M+H).

### Example 9

### 2-[4,4-Bis-(4-fluoro-phenyl)-butyl]-1,2,3,4-tetrahydro-isoquinoline

Prepared according to the method described in Example 8. HPLC retention time 8.29 min. Mass Spectrum (ES+) m/z 378(M+H).

### Example 10

### 2-[4,4-Bis-(4-fluoro-phenyl)-butyl]-8-methoxy-1,2,3,4-tetrahydro-isoquinoline

Prepared according to the method described in Example 8. HPLC retention time 8.39 min. Mass Spectrum (ES+) m/z 408(M+H).

### Example 11

### 2-(2,2-Diphenyl-ethyl)-1,2,3,4-tetrahydro-isoquinoline-8-ol

To a stirred solution of 1,2,3,4-Tetrahydro-isoquinolin-8-ol acetate salt (0.120 g, 0.57 mmol) in CH₃OH (5 mL) was added Et₃N (Aldrich 47,128-3) (0.058 g, 0.57 mmol). The reaction mixture was stirred for 0.5 h, diphenylacetaldehyde (Aldrich D20-245-0) (0.113 g, 0.57 mmol) in CH₃OH (5 mL), and sodium cyanoborohydride (Aldrich 15,615-9) (0.036 g, 0.57 mmol) was added. The reaction mixture was stirred for 18 h. The solvent was removed in vacuo and the residue was purified by flash column chromatography using CH₂Cl₂/CH₃OH, 95/5 vv to afford the title compound as a white solid (0.032 g, 17%). HPLC retention time 3.21 min. Mass Spectrum (ES+) m/z 330(M+H).

### Example 12

### 2-(2,2-Diphenyl-ethyl)1,2,3,4-tetrahydro-isoquinoline

Prepared according to the method described in Example 11 but with CH₂Cl₂ as the reaction solvent. HPLC retention time 4.96 min. Mass Spectrum (ES+) m/z 314(M+H).

### Example 13

### 2-(2,2-Diphenyl-ethyl)-8-methoxy-1,2,3,4-tetrahydro-isoquinoline

Prepared according to the method described in Example 11 but with CH₂Cl₂ as the reaction solvent. HPLC retention time 4.96 min. Mass Spectrum (ES+) m/z 344(M+H).

### Example 14

### N-Benzyhydryl-2-(3,4-dihydro-1H-isoquinolin-2-yl)-acetamide

To a stirred solution of 1,2,3,4-Tetrahydroisoquinoline (Aldrich A5,5560-8) (0.133 g, 1 mmol) in MeCN (15 mL) was added-potassium carbonate (Acros P/4120/50) 0.138 g, 1 mmol)), tetrabutlylammonium iodide (Aldrich 14,077-5) (0.074 g, 0.02 mmol). To this suspension was added N-Benzhydryl-2-chloro-acetamide (0.259 g, 1 mmol) in MeCN (10 mL). The reaction mixture was heated at reflux for 8 h, cooled to room temperature and filtered. The solvent was removed in vacuo and the residue purified by flash column chromatography using iso-hexane.EtOAc as mobile phase to afford the title compound as a clear oil (0.256 g, 72%). HPLC retention time 4. Mass Spectrum (ES+) m/z 357(M+H).

### Example 15

### 2-(3,4-Dihydro-1H-isoquinolin-2-yl)-N-(9H-fluorenyl)-acetamide

Prepared according to the method described in Example 14. HPLC retention time 4.34 min. Mass Spectrum (ES+) m/z 355(M+H).

### Example 16

### N,N-Dibenzyl-2-(3,4-dihydro-1H-isoquinolin-2yl)-acetamide

Prepared according to the method described in Example 14. HPLC retention time 4.41 min. Mass Spectrum (ES+) m/z 371(M+H).

### Example 17

### N-Benzyl-2-(3,4-dihydro-1H-isoquinolin-2-yl)-N-phenyl-acetamide

Prepared according to the method described in Example 14. HPLC retention time 4.24 min. Mass Spectrum (ES+) m/z 357(M+H).

### Example 18

### 2-(3,4-Dihydro-1H-isoquinolin-2yl)-N-(3,3-diphenyl-propyl)-acetamide

Prepared according to the method described in Example 14. HPLC retention time 4.35 min. Mass Spectrum (ES+) m/z 385(M+H).

### Example 19

### N-Benzhydryl-2-(8-methoxy-3,4-dihydro-1H-isoquinolin-2-yl)-acetamide

Prepared according to the method described in Example 14. HPLC retention time 4.30 min. Mass Spectrum (ES+) m/z 387(M+H).

### Example 20

### N-(9H-Fluorenyl-9-yl)-2-(8-methoxy-3,4-dihydro-1H-isoquinoline-2-yl)-acetamide

Prepared according to the method described in Example 14. HPLC retention time 4.20 min. Mass Spectrum (ES+) m/z 385(M+H).

### Example 21

### N-Benzhydryl-2-(8-methoxy-3,4-dihydro-1H-isoquinolin-2-yl)-N-phenyl-acetamide

Prepared according to the method described in Example 14. HPLC retention time 4.15 min. Mass Spectrum (ES+) m/z 387(M+H).

### Example 22

### N-(3,3-Diphenyl-propyl)-2-(8-methoxy-3,4-dihydro-1H-isoquinolin-2-yl)-acetamide

Prepared according to the method described in Example 14. HPLC retention time 4.22 min. Mass Spectrum (ES+) m/z 415(M+H).

### Example 23

### N,N-Dibenzyl-2-(8-hydroxy-3,3-dihydro-1H-isoquinolin-2-yl)-acetamide

Prepared according to the method described in Example 14. HPLC retention time 4.21 min. Mass Spectrum (ES+) m/z 387(M+H).

### Example 24

### N-Benzhydryl-2-(8-hydroxy-3,4-dihydro-1H-isoquinolin-2-yl)-acetamide

Prepared according to the method described in Example 14. HPLC retention time 4.03 min. Mass Spectrum (ES+) m/z 373(M+H).

### Example 25

### N-Benzyl-2-(8-hydroxy-3,4-dihydro-1H-isoquinolin-2-yl)-N-phenyl-acetamide

Prepared according to the method described in Example 14. HPLC retention time 3.99 min. Mass Spectrum (ES+) m/z 373(M+H).

### Example 26

### N-(9H-fluoren-9-yl)-2-(8-hydroxy-3,4-dihydro-1H-isoquinolin-2-yl)acetamide

Prepared according to the method described in Example 14. HPLC retention time 4.02 min. Mass Spectrum (ES+) m/z 371(M+H).

### Example 27

### N-(3,3-Diphenyl-propyl)-2-(8-hydroxy-3,4-dihydro-1H-isoquinolin-2-yl)-acetamide

Prepared according to the method described in Example 14. HPLC retention time 4.10 min. Mass Spectrum (ES+) m/z 401(M+H).

### Example 28

### 2-(3,4-Dihydro-1H-isoquinolin-2-yl)-N-[1-(5-methyl-thiazol-2-yl)-ethyl]-acetamide

Prepared according to the method described in Example 14. HPLC retention time 3.73 min. Mass Spectrum (ES+) m/z 316(M+H).

### Example 29

### 2-(8-Hydroxy-3,4-dihydro-1H-isoquinolin-2-yl)-N-[1-(5-methyl-thiazol-2-yl)-ethyl]-acetamide

Prepared according to the method described in Example 14. HPLC retention time 3.21 min. Mass Spectrum (ES+) m/z 332(M+H).

### Example 30

### 1-(3,4-Dihydro-1H-isoquinoline-2-yl-3,3-bis-(4-fluoro-phenyl)-propan-1-one

To a stirred solution of 1,2,3,4-Tetrahydroisoquinoline (Aldrich A5,5560-8) (0.102 g, 0.76 mmol) in CH₂Cl₂ (5 mL) was added 3,3-Bis-(4-fluro-phenyl)-propionyl chloride (0.107 g, 0.33 mmol). The reaction mixture was stirred for 5 h and the solvent removed in vacuo. The residue was purified by flash column chromatography using CH₂Cl₂ as mobile phase followed by preparative HPLC to give the title compound as an oil (3.4 mgs, (2%). HPLC retention time 4.39 min. Mass Spectrum (ES+) m/z 378(M+H).

### Example 31

### 2-(Benzhydryl-amino)-1-(3,4-dihydro-1H-isoquinolin-2-yl)-ethanone

To a stirred solution of 2-Chloro-1-(3,4-dihydro-1H-isoquinolin-2-yl)-ethanone (0.150 g, 0.71 mmol) in acetonitril was added of aminodiphenylmethane (Aldrich A5,360-5) (0.131 g, 0.71 mmol), tetrabutlylammonium iodide (Aldrich 14,077-5) (0.53 g, 0.14 mmol) and potassium carbonate (Acros) (0.99 g, 0.71 mmol). The reaction mixture was heated at reflux for 5 h and cooled to room temperature and the solvent removed in vacuo. The residue was purified by column chromatography using EtOAc/iso-hexane, 1/1, v/v, to give the title compound as a colourless oil (0.10 g, 39%). HPLC retention time 6.65 min. Mass Spectrum (ES+) m/z 357(M+H).

### Example 32

### 1-(3,4-Dihydro-1-isoquinolin-2-yl)-2-(2,2-diphenyl-ethylamino)-ethanone

To a stirred solution of 2-Chloro-1-(3,4-dihydro-1H-isoquinolin-2-yl)-ethanone (0.209 g, 11.0 mmol) was added (0.197 g, 1.0 mmol), 2,2-Diphenylpropyamine (Aldrich D20-670-9)(0.2113 g, 11.0 mmol) tetrabutlylammonium iodide (Aldrich 14,077-5) (0.369 g, 0.074 mmol) and potassium carbonate (Acros) (0.99 g, 0.71 mmol). The reaction mixture was heated at reflux for 18 h, cooled to room temperature and the solvent removed in vacuo. The residue was purified by column chromatography using EtOAc/iso-hexane, 1/3, v/v, to give the title compound as a colourless oil (0.047 g, 12%). HPLC retention time 4.24 min. Mass Spectrum (ES+) m/z 385(M+H).

### Example 33

### 1-(3,4-Dihydro-1H-isoquinolin-2-yl)-2-[[2-(3,4-dihydro-1H-isoquinolin-2-yl)-2-oxo-ethyl]-(3,3-diphenyl-propyl)-amino]-ethanone

Prepared according to the method described in Example 31. HPLC retention time 4.70 min. Mass Spectrum (ES+) m/z 558(M+H).

### Example 34

### 1-(3,4-Dihydro-1H-isoquinolin-2-yl)-2-(3,3-diphenyl-propylamino)-ethanone

Prepared according to the method described in Example 31. HPLC retention time 4.30 min. Mass Spectrum (ES+) m/z 385(M+H).

### Example 35

### 2-Dibenzylamino-1-(3,4-dihydro-1H-isoquinolin-2-yl)-ethanone

Prepared according to the method described in Example 31. HPLC retention time 4.72 min. Mass Spectrum (ES+) m/z 371(M+H).

### Example 36

### 2-{(2,2-Diphenyl-ethyl)-[2-8-methoxy-3,4-dihydro-1H-isoquinolin-2-yl)-2-oxo-ethyl]-amino}-1H-isoquinolin-2-yl)-ethanone

Prepared according to the method described in Example 31. HPLC retention time 4.75 min. Mass Spectrum (ES+) m/z 604(M+H).

### Example 37

### 2-{Benzhydryl-[2-(8-methoxy-3,4-dihydro-1H-isoquinolin-2-yl)-2-oxo-ethyl]-amino}-1-(8-methoxy-3,4-dihydro-1H-isoquinolin-2-yl)-ethanone

Prepared according to the method described in Example 31. HPLC retention time 7.57 min. Mass Spectrum (ES+) m/z 560(M+H).

### Example 38

### 2-(Benzhydryl-amino)-1-(8-methoxy-3,4-dihydro-1H-isoquinoline-2-yl)-ethanone

Prepared according to the method described in Example 31. HPLC retention time 6.18 min. Mass Spectrum (ES+) m/z 387(M+H).

### Example 39

### 2-(2,2-Diphenyl-ethylamino)-1-(8-methoxy-3,4-dihydro-1H-isoquinolin-2-yl)-ethanone

Prepared according to the method described in Example 31. HPLC retention time 6.65 min. Mass Spectrum (ES+) m/z 401(M+H).

### Example 40

### 2-(1,3-Dihydro-isoindol-2-yl)-N-(3,3-diphenyl-propyl)-acetamide

To a stirred solution of isoindoline (Aldrich 51,557-4) (0.25 g, 2.1 mmol) in MeCN (15 mL) was added 2-Chloro-N-(3,3-diphenyl-propyl)-acetamide (0.60 g, 2.1 mmol), tetrabutlylammonium iodide (Aldrich 14,077-5) (0.16 g, 0.42 mmol) and Et₃N) (Aldrich 47,128-3) (600 μl, 2.1 mmol). The reaction mixture was heated at reflux for 4 h, and cooled to room temperature, and the solvent removed in vacuo. The residue was dissolved in CH₂Cl₂ (100 mL), washed with H₂O (20 mL), dried (Na₂SO₄) and the solvent remove in vacuo. The residue was purified by flash column chromatography using EtOAc/iso-hexane, 1/1 as mobile phase to give the title compound as a tan solid (0.25 g, 32%). HPLC retention time 4.33 min. Mass Spectrum (ES+) m/z 371(M+H).

### Example 41

### N-Benzhydryl-2-(1,3-dihydro-isoindol-2-yl)-acetamide

Prepared according to the method described in Example 40. HPLC retention time 4.32 min. Mass Spectrum (ES+) m/z 343(M+H).

### Example 42

### 2-Benzhydrylideneaminooxy-1-(3,4-dihydro-1H-isoquinoline-2-yl)-ethanone

To a suspension of sodium hydride 60% dispersion in mineral oil (Aldrich 2,344-1) in dimethyl foramide (2 mL) cooled in an ice bath was added benzophenone oxime (Lancaster 0817) (0.47 g, 2.39 mmol). The reaction mixture was removed from the ice bath and stirred at room temperature for 0.5 h. To this solution was added 2-Chloro-1-(3,4-dihydro-1H-isoquinolin-2-yl)-ethanone (0.5 g, 2.39 mmol) in dimethyl formamide (1 mL). The reaction was stirred for 18 h, diluted with H₂O (30 mL), extracted with Et₂O (50 mL), dried (Na₂SO₄) and the solvent removed in vacuo. The residue was purified by preparative HPLC (Solvent: MeCN/H₂O/0.05% NH₃, 5-95% gradient H₂O-6 min. Column: Phenomenex 50×4.6 mm i.d., C18 reverse phase. Flow rate: 15 mL/min.) to give the title compound as a glass (0.44 g, 55%). HPLC retention time 4.53 min. Mass Spectrum (ES+) m/z 371(M+H).

### Example 43

### 2-Benzhydrylideneaminooxy-1-(8-methoxy-3,4-dihydro-1H-isoquinoline-2-yl)-ethanone

Prepared according to the method described in Example 42. HPLC retention time 4.48 min. Mass Spectrum (ES+) m/z 401(M+H).

### Example 44

### 2-(Di-pyridin-2-yl-methyleneaminooxy)-1-(8-methoxy-3,4-dihydro-1H-isoquinoline-2-yl)-ethanone

Prepared according to the method described in Example 42. HPLC retention time 3.50 min. Mass Spectrum (ES+) m/z 403(M+H).

### Example 45

### 2-(5-Phenyl-2H-[1,2,3]triazol-4-ylmethyl)-1,2,3,4-tetrahydro-isoquinolin-8-ol

### 5-Phenyl-2H-[1,2,3]-triazole-4-carbaldehyde

To a stirred solution of phenylacetylene (Aldrich 11,770-6) (5.1 g, 50 mmol) in anhydrous tetrahydrofuran (125 mL) at −40° C. under nitrogen was added dropwise over c.a 2 min nButyl lithium (Aldrich 18,617-1) (31.3 mL, 0.50 mmol) whilst maintaining the temperature (internal) between −35° C. to −40° C. with external cooling. To this solution was added anhydrous dimethyl formamide (7.75 mL) and the reaction mixture allowed to warn to room temperature, stirred for 0.5 h and quenched by pouring into a rapidly stirred biphasic solution of 10% potassium dihydrogen phosphate (270 mL) and methyl tert-butyl ether (250 mL), cooled to c.a. −5° C. The layers were separated and the aqueous layer back extracted with methyl tert-butyl ether (100 mL). The combined organic layers were washed with H₂O (2×200 mL), dried (MgSO₄) and evaporated to dryness in vacuo to give a yellow oil which was purified by flash column chromatography to give 6.1 g of a pale yellow oil. A solution of this oil (3.1 g in dimethyl sulphoxide (17.5 mL) was added to a vigorously stirred solution of sodium azide (Aldrich 19,993-1) (1.79 g, 27.5 mmol) over c.a. 10 min whilst maintaining the temperature (internal) between 20 to 25° C. The reaction mixture was stirred for a further 0.5 h and quenched by pouring into a rapidly stirred biphasic solution of 15% potassium dihydrogen phosphate (150 mL) and methyl tert-butyl ether (160 mL). The organic layer was separated and washed with H₂O (2×100 mL). The aqueous layers were re-extracted with methyl tert-butyl ether (100 mL) and the combined organic layers dried over (MgSO₄) and evaporated in vacuo to afford the title compound as an off white solid (3.1 g, 65%): ¹H NMR (400 MHz, CDCl₃) δH 7.46-7.59 (3H), 7.66-7.89 (2H), 10.14 (1H), 16.08 (1H).

### 2-(5-Phenyl-2H-[1,2,3]triazol-4-ylmethyl)-1,2,3,4-tetrahydro-isoquinolin-8-ol

To a stirred solution of 1,2,3,4-Tetrahydro-isoquinolin-8-ol acetate salt (0.120 g, 0.57 mmol) in CH₃OH (5 mL) was added Et₃N (Aldrich 47,128-3) (0.058 g, 0.57 mmol). The reaction mixture was stirred for 0.5 h, 5-phenyl-2H-[1,2,3]-triazole-4-carbaldehyde (0.025 g, 0.14 mmol) in CH₃OH (5 mL), and sodium cyanoborohydride (Aldrich 15,615-9) (0.009 g, 0.14 mmol) was added. The reaction mixture was heated at reflux for 5 h, cooled to room temperature and the solvent removed in vacuo. The residue was purified by flash column chromatography using EtOAc/iso-hexane 1/1, v/v as mobile phase to afford the title compound as a viscous oil (0.004 g, 10%). HPLC retention time 2.54 min. Mass Spectrum (ES+) m/z 306(M+H).

### Example 46

### [2-(3,4-Dihydro-1H-isoquinolin-2-yl)-ethyl]-(3,3-diphenyl-propyl)-amine

To a stirred solution of 2-(3,4-Dihydro-1H-isoquinolin-2yl)-N-(3,3-diphenyl-propyl)-acetamide: (0.184 g, 0.047 mmol) in tetrahydrofuran (10 mL) was added lithium aluminium hydride 1M in Et₂O (Aldrich 21,279-2) (10 mL, 10 mmol). The reaction mixture was heated at reflux 8 h, cooled to room temperature and stirred for 18 h. The reaction mixture was quenched with CH₂Cl₂ (30 mL) and sodium hydroxide solution (2M, 4 mL). The CH₂Cl₂ layer was separated, washed with H₂O dried (Na₂SO₄) and the solvent removed in vacuo. The residue was purified by preparative HPLC (Solvent: MeCN/H₂O/0.05% NH₃, 5-95% gradient H2O-10 min. Column: Phenomenex 50×19 mm i.d., C18 reverse phase. Flow rate: 15 mL/min.), to give the title compound as a pale yellow oil (0.007 g, 3.9%). HPLC retention time 7.76 min. Mass Spectrum (ES+) m/z 371(M+H).

### Example 47

### Dibenzyl-[2-(3,4-dihydro-1H-isoquinolin-2-yl)-ethyl]-amine

Prepared according to the method described in Example 46. HPLC retention time 8.48 min. Mass Spectrum (ES+) m/z 357(M+H).

### Example 48

### 2-(2-Benzyloxy-propyl)-1,2,3,4-tetrahydro-isoquinolin-8-ol

To a stirred solution of 2-Benzyloxypropionic acid (0.318 g, 1.76 mmol) in CH₂Cl₂ (3 mL) was added oxalyl chloride (Aldrich O-880-1) (1.12 g, 8.83 mmol). The reaction mixture was stirred at room temperature for 5 h and the solvent and excess reagent removed in vacuo. The residue was dissolved in CH₂Cl₂ (2 mL) and added to a stirred solution of 1,2,3,4-Tetrahydro-isoquinolin-8-ol acetate salt (0.367 g, 3.52 mmol), Et₃N (Aldrich 47,128-3) (0.356 g, 3.52 mmol) in CH₂Cl₂ (2 mL) and the reaction mixture was stirred overnight. The reaction mixture was diluted with 5% hydrochloric acid (5 mL), separated and the organic layer washed with H₂O (5 mL), brine (5 mL), dried, (Na₂SO₄) and the solvent remove in vacuo. The residue (0.147 g) was dissolved in tetrahydrofuran (2 mL) and Lithium aluminium hydride (Aldrich 21,277-6) (1M in THF, 1 mL, 1 mmol). The reaction mixture was heated at reflux for 2 h, cooled to room temperature and diluted with CH₂Cl₂ (10 mL). The mixture was extracted with H₂O (5 mL×2), brine (5 mL), dried (Na₂SO₄), filtered and the solvent removed in vacuo. The residue was purified by flash column chromatography to afford the title compound as a oil (0.073 g, 0.52%). HPLC retention time 3.11 min. Mass Spectrum (ES+) m/z 298 (M+H).

### Example 49

### 4-Methoxy-1,3-dihydro-1H-isoindole-2-carbothioic acid benzhydryl-amide

### 2-Benzyl-4-methoxy-2,3-dihydro-2H-isoindole

2,3-Dimethylanisole (Acros, 15999) (12.5 g, 91.8 mmol), N-bromosuccinimide (Aldrich, B8,125-5) (32.6 g, 183.5 mmol) and benzoyl peroxide (Lancaster, 13174) (300 mg) were refluxed in CCl₄ (200 mL) for 20 hrs. The reaction was cooled and the insoluble material removed by filtration. The solid was washed with CCl₄ and the combined filtrate concentrated in vacuo to afford a yellow solid which was used without further purification. The yellow solid and benzyltriethylammonium chloride (Acros, 16402) (0.75 g, 3.3 mmol) were dissolved in a mixture of 50% aqs NaOH (40 mL) and toluene (175 mL). To the solution was added drop-wise, benzylamine (Aldrich, 18,570-1) (91.8 g, 101 mmol) over 15 mins at ambient temperature. Once addition was complete, the reaction was stirred for 24 hrs at rt. The organic layer was separated, washed with brine (3×100 mL), dried (MgSO₄) and concentrated in vacuo. The residue was purified via flash chromatography, eluting with EtOAc/isohexane (1:15) to afford 2-benzyl-4-methoxy-2,3-dihydro-1H-isoindole as a red oil. Yield 6.5 g (30%). HPLC retention time 4.21 min. Mass spectrum (ES+) m/z 240 (M+H).

### 4-Methoxy-2,3-dihydro-1H-isoindole

2-Benzyl-4-methoxy-2,3-dihydro-1H-isoindole (1.9 g, 7.94 mmol) was dissolved in CH₃OH (50 mL) and placed in a 250 mL autoclave. 10% Palladium on activated charcoal (Acros, 19503) (300 mg) was added and the reaction was hydrogenated at 3.5 bar for 24 hrs. When complete, the catalyst was separated via filtration, and the solvent was removed in vacuo. The residue was purified via flash chromatography eluting with MeOH/CH₂Cl₂ (1:4) to afford 4-methoxy-2,3-dihydro-1H-isoindole as a beige solid. Yield 0.720 g (61%). HPLC retention time, 3.07 min. Mass spectrum (ES+) m/z 150 (M+H).

### 4-Methoxy-1,3-dihydro-1H-isoindole-2-carbothioic acid benzhydryl-amide

2-Benzyl-4-methoxy-2,3-dihydro-1H-isoindole (50 mg, 0.335 mmol) and benzhydryl isothiocyanate (Fluorochem, 18194) (75 mg, 0.335 mmol) were stirred in toluene (2 mL) for 24 hrs at ambient temperature. The solvent was removed in vacuo and the residue was purified via flash chromatography eluting with EtOAc/isohexane (1:4) to afford the title compound as a white solid. Yield 95 mg (76%). HPLC retention time 4.50 min. Mass spectrum (ES+) m/z 375 (M+H).

### Example 50

### 3,4-Dihydro-1H-isoquinoline-2-carbothioic acid benzhydryl-amide

Prepared according to the method described in Example 49. HPLC retention time, 4.49 min. Mass spectrum (ES+) m/z 359 (M+H).

### Example 51

### 3,4-Dihydro-1H-isoquinoline-2-carbothioic acid (2,2-diphenyl-ethyl)-amide

Prepared according to the method described in Example 49: HPLC retention time, 4.59 min. Mass spectrum (ES+) m/z 373 (M+H).

### Example 52

### 8-Methoxy-3,4-dihydro-1H-isoquinolin-2-carbothioic acid (2,2-diphenyl-ethyl)-amide

Prepared according to the method described in Example 49. HPLC retention time 4.53 min. Mass spectrum (ES+) m/z 403 (M+H).

### Example 53

### 3,4-Dihydro-1H-isoquinoline-2-carbothioic acid benzhydryl-amide

Prepared according to the method described in Example 49. HPLC retention time 4.51 min. Mass spectrum (ES+) m/z 389 (M+H).

### Example 54

### 7-Methoxy-1,3,4,5-tetrahydro-benzo[c]azepin-2-carbothioic acid benzhydryl-amide

Prepared according to the method described in Example 49. HPLC retention time 4.46 min. Mass spectrum (ES+) m/z 403 (M+H).

### Example 55

### 7-Methoxy-1,3,4,5-tetrahydro-benzo[c]azepin-2-carbothioic acid (2,2-diphenyl-ethyl)-amide

Prepared according to the method described in Example 49. HPLC retention time 4.53 min. Mass spectrum (ES+) m/z 417 (M+H).

### Example 56

### Example 2

### N-Benzhydryl-2-(4-methoxy-1,3-dihydro-isoindol-2-yl)-acetamide

A solution of 2-benzyl-4-methoxy-2,3-dihydro-1H-isoindole (75 mg, 0.50 mmol), K₂CO₃ (69 mg, 0.50 mmol) and tetrabutylammonium iodide (Aldrich, 14,077-5) (37 mg, 0.1 mmol) in MeCN (3 mL) was stirred at rt for 30 mins. N-Benzhydryl-2-chloro-acetamide (130 mg, 0.5 mmol) was added and the reaction was refluxed for 5 hrs. The reaction mixture was allowed to cool, diluted with MeCN (5 mL), and the solids removed by filtration. The solvent was removed in vacuo and the residue purified via flash chromatography eluting with EtOAc/isohexane (1:2) to afford the title compound as a pale green solid. Yield 60 mg (32%). HPLC retention time 4.24 min. Mass spectrum (ES+) m/z 373 (M+H).

### Example 57

### N-(2,2-Diphenyl-ethyl)-2-(4-methoxy-1,3-dihydro-isoindol-2-yl)-acetamide

Prepared according to the method described in Example 56. HPLC retention time 3.10 min (Solvent: MeCN/H₂O/0.05% HCOOH, 5-95% gradient H₂O-6 min. Column: Xterra 50×4.60 i.d., C18 reverse phase. Flow rate: 1.5 mL/min.). Mass spectrum (ES+) m/z 387 (M+H).

### Example 58

### N-(3,3-Diphenyl-propyl)-2-(4-methoxy-1,3-dihydro-isoindol-2-yl)-acetamide

Prepared according to the method described in Example 56. HPLC retention time 4.32 min. Mass spectrum (ES+) m/z 401 (M+H).

### Example 59

### N-(4,4-Diphenyl-butyl)-2-(4-methoxy-1,3-dihydro-isoindol-2-yl)-acetamide

Prepared according to the method described in Example 56. HPLC retention time 4.41 min. Mass spectrum (ES+) m/z 415 (M+H).

### Example 60

### 2-(3,4-Dihydro-1H-isoquinolin-2-yl)-N-(2,2-diphenyl-ethyl)-acetamide

Prepared according to the method described in Example 56. HPLC retention time 6.71 min. Mass spectrum (ES+) m/z 371 (M+H).

### Example 61

### N-(2,2-Diphenyl-ethyl)-2-(8-methoxy-3,4-dihydro-1H-isoquinolin-2-yl)-acetamide

Prepared according to the method described in Example 56. HPLC retention time 4.57 min. Mass spectrum (ES+) m/z 401 (M+H).

### Example 62

### 1-(4-benzhydryl-piperazin-1-yl)-2-(8-methoxy-3,4-dihydro-1H-isoquinolin-2-yl)-ethanone

### 1-(4-Benzhydryl-piperazin-1-yl)-2-chloro-ethanone

A solution of 1-benzhydryl-piperazine (Acros, 12293) (5.05 g, 20 mmol) and Et₃N (2.22 g, 22 mmol) in CH₂Cl₂ (20 mL) was cooled to 5° C. using an ice/H₂O cooling. Chloroacetyl chloride (Aldrich, 10,449-3) (2.5 g, 22 mmol) in CH₂Cl₂ (5 mL) was added drop wise such that the temperature remained below 20° C. Once addition was complete, the reaction was stirred for a further 18 hrs at ambient temperature. Deionised H₂O (50 mL) was added and stirring continued for a further 1 hr. The organic layer was separated, washed with brine (3×100 mL), dried (MgSO₄) and concentrated in vacuo to afford 1-(4-benzhydryl-piperazin-1-yl)-2-chloro-ethanone as a brown oil, which was used without further purification. Yield 6.8 g (95%). HPLC retention time, 4.22 min. Mass spectrum (ES+) m/z 329 (M+H).

### 1-(4-Benzhydryl-piperazin-1-yl)-2-(8-methoxy-3,4-dihydro-1H-isoquinolin-2-yl)-ethanone

Prepared according to the method described in Example 55. HPLC retention time, 4.77 min. Mass spectrum (ES+) m/z 456 (M+H).

### Example 63

### 1-{4-[Bis-(4-fluoro-phenyl)-methyl]-piperazin-1-yl}-2-(8-methoxy-3,4-dihydro-1H-isoquinolin-2-yl)-ethanone

### 1-{4-[Bis-(4-fluoro-phenyl)-methyl]-piperazin-1-yl}-2-chloro-ethanone

Prepared according, to the method described in Example 62. HPLC retention time 4.26 min. Mass spectrum (ES+) m/z 365 (M+H).

### 1-{4-[Bis-(4-fluoro-phenyl)-methyl]-piperazin-1-yl}-2-(8-methoxy-3,4-dihydro-1H-isoquinolin-2-yl)-ethanone

Prepared according to the method described in Example 55. HPLC retention time 4.74 min. Mass spectrum (ES+) m/z 492 (M+H).

### Example 64

### 1-(4-Benzhydryl-piperazin-1-yl)-2-(3,4-dihydro-1H-isoquinolin-2-yl)-ethanone

Prepared according to the method described in Example 56. HPLC retention time 4.71 min. Mass spectrum (ES+) m/z 426 (M+H).

### Example 65

### 1-{4-[Bis-(4-fluoro-phenyl)-methyl]-piperazin-1-yl}-2-(3,4-dihydro-1H-isoquinolin-2-yl)-ethanone

Prepared according to the method described in Example 56. HPLC retention time 4.66 min. Mass spectrum (ES+) m/z 461 (M+H).

### Example 66

### 2-(1,3-Dihydro-isoindol-2-yl)-N-(2,2-diphenyl-ethylacetamide

Prepared according to the method described in Example 56. HPLC retention time 4.28 min. Mass spectrum (ES+) m/z 357 (M+H).

### Example 67

### 1-(4-Benzhydryl-piperazin-1-yl)-2-(1,3-dihydro-isoindol-2-yl)-ethanone

Prepared according to the method described in Example 56. HPLC retention time 4.50 min. Mass spectrum (ES+) m/z 412 (M+H).

### Example 68

### 1-{4-[Bis-(4-fluoro-phenyl)-methyl]-piperazin-1-yl}-2-(1,3-dihydro-isoindol-2-yl)-ethanone

Prepared according to the method described in Example 56. HPLC retention time 4.52 min. 1H NMR (400 MHz (CD₃)₂SO)□_{H} 2.20-2.25 (4H), 3.40-3.55 (6H), 3.90 (4H), 4.40 (1H), 7.05-7.20 (8H), 7.35-7.45 (4H). Mass spectrum (ES+) m/z 448 (M+H).

### Example 69

### 2-(8-methoxy-3,4-dihydro-1H-isoquinolin-2-yl)-N-(phenyl-pyridin-2-yl-methyl)-acetamide

### 2-Chloro-N-(phenyl-pyridin-2-yl-methyl)-acetamide

Prepared according to the method described in Example 1. Yield 600 mg (98%). HPLC retention time 3.40 min. Mass spectrum (ES+) m/z 261 (M+H).

### 2-(8-methoxy-3,4-dihydro-1H-isoquinolin-2-yl)-N-(phenyl-pyridin-2-yl-methyl)-acetamide

Prepared according to the method described in Example 56. HPLC retention time 4.15 min. Mass spectrum (ES+) m/z 388 (M+H).

### Example 70

### 2-(8-Hydroxy-3,4-dihydro-1H-isoquinolin-2-yl)-1-(3-phenyl-2,3-dihydro-benzo[1,4]oxazin-4-yl)-ethanone

### 2-(2-Nitro-phenoxy)-1-phenyl-ethanone

A solution of 2-nitrophenol (Aldrich, N1,970-2) (13.9 g, 100 mmol) and K₂CO₃ (15.2 g, 10 mmol) was stirred in MeCN (50 mL) at rt for 30 mins. KI (1.83 g, 11 mmol) was added in one portion followed by phenacyl bromide (Lancaster, 6260) (19.9 g, 100 mmol) in portions. After addition the reaction was stirred for 24 hrs at RT, and poured onto ice/H2O (1 ltr) with stirring. The solid was separated via filtration and washed with H2O. The solid was dried and recrystallized ex IPA (300 mL) to afford 2-(2-nitro-phenoxy)-1-phenyl-ethanone as cream coloured crystals. Yield 20 g (80%). HPLC retention time 3.83 min. Mass spectrum (ES+) m/z 258 (M+H).

### 3-Phenyl-3,4-dihydro-2H-benzo[1,4]oxazine

To a stirred solution of sodium hypophosphite (Aldrich, 24,366-3) (50 g) in deionised H2O (200 mL) and THF (200 mL) containing 2-(2-nitro-phenoxy)-1-phenyl-ethanone (10 g, 39 mmol) was added 10% Palladium on activated charcoal (Acros, 19503) (1 g). The reaction was stirred at RT for 18 hrs sodium hypophosphite (Aldrich, 24,366-3) (50 g) and 10% Palladium on activated charcoal (Acros, 19503) (1 g) was added and the reaction was stirred for a further 18 hrs at RT. The catalyst was filtered off and the two phase mixture was diluted with deionised H₂O and extracted with Et₂O (×3). The combined extracts were washed with H2O and dried over MgSO₄. The solvent was removed in vacuo to afford 3-phenyl-3,4-dihydro-2H-benzo[1,4]oxazine as a red oil which was used without further purification. Yield 8.2 g (100%).

### 2-Chloro-1-(3-phenyl-2,3-dihydro-benzo[1,4]oxazin-4-yl)-ethanone

Prepared according to the method described in Example 62. HPLC retention time 3.91 min (Solvent: MeCN/H₂O/0.05% HCOOH, 5-95% gradient H₂O-6 min. Column: Xterra 50×4.60 i.d., C18 reverse phase. Flow rate: 1.5 mL/min.). 1H NMR (400 MHz (CD₃)₂SO)□_{H} 4.45-4.55 (2H), 4.80 (1H), 4.95 (1H), 5.80 (1H), 6.80 (1H), 6.90 (1H), 7.00 (1H), 7.20-7.25 (1H), 7.30-7.35 (4H), 7.80 (1H). Mass spectrum (ES+) m/z 288 (M+H).

### 2-(8-Hydroxy-3,4-dihydro-1H-isoquinolin-2-yl)-1-(3-phenyl-2,3-dihydro-benzo[1,4]oxazin-4-yl)-ethanone

Prepared according to the method described in Example 56. HPLC retention time 6.30 min (Solvent: MeCN/H₂O/0.05% NH₃, 5-95% gradient H₂O-10 min. Column: Xterra 50×4.60 i.d., C18 reverse phase. Flow rate: 1.5 mL/min.). 1H NMR (400 MHz (CD₃)₂SO)□_{H} 2.60-2.70 (4H), 3.45-3.65 (4H), 4.35 (1H), 4.90 (1H), 5.95 (1H), 6.50 (1H), 6.55 (1H), 6.75 (1H), 6.85-6.90 (2H), 6.95-7.00 (1H), 7.15 (1H), 7.20-7.30 (4H), 8.00 (1H), 9.30 (1H). Mass spectrum (ES+) m/z 401 (M+H).

### Example 71

### 2-(8-Methoxy-3,4-dihydro-1H-isoquinolin-2-yl)-1-(3-phenyl-2,3-dihydro-benzo[1,4]oxazin-4-yl)-ethanone

Prepared according to the method described in Example 56. HPLC retention time 4.49 min. Mass spectrum (ES+) m/z 415 (M+H).

### Example 72

### 2-(8-Methoxy-3,4-dihydro-1H-isoquinolin-2-yl)-1-phenoxazin-10-yl-ethanone

Prepared according to the method described in Example 56. HPLC retention time 4.53 min. Mass spectrum (ES+) m/z 387 (M+H).

### Example 73

### 1-(10,11-Dihydro-dibenzo[b,f]azepin-5-yl)-2-(8-methoxy-3,4-dihydro-1H-isoquinolin-2-yl)-ethanone

Prepared according to the method described in Example 56. HPLC retention time 4.37 min. Mass spectrum (ES+) m/z 399 (M+H).

### Example 74

### N,N-Dibenzyl-2-(8-methoxy-3,4-dihydro-1H-isoquinolin-2-yl)-acetamide

Prepared according to the method described in Example 56. HPLC retention time 4.57 min. Mass spectrum (ES+) m/z 401 (M+H).

### Example 75

### N,N-Diisopropyl-2-(8-methoxy-3,4-dihydro-1H-isoquinolin-2-yl)-acetamide

Prepared according to the method described in Example 56. HPLC retention time 4.26 min. Mass spectrum (ES+) m/z 305 (M+H).

### Example 76

### N-(4,4-Diphenyl-butyl)-2-(8-methoxy-3,4-dihydro-1H-isoquinolin-2-yl)-acetamide

Prepared according to the method described in Example 56. HPLC retention time 4.55 min. Mass spectrum (ES+) m/z 429 (M+H).

### Example 77

### N-(3,3-Diphenyl-propyl)-2-(8-methoxy-3,4-dihydro-1H-isoquinolin-2-yl)-propionamide

Prepared according to the method described in Example 56 with the following modification: the reaction was refluxed for 24 hrs. HPLC retention time 4.36 min. Mass spectrum (ES+) m/z 429 (M+H).

### Example 78

### N,N-Dibenzyl-3-(8-methoxy-3,4-dihydro-1H-isoquinolin-2-yl)-propionamide

Prepared according to the method-described in Example 56 with the following modification: the reaction was refluxed for 24 hrs. HPLC retention time 4.45 min. Mass spectrum (ES+) m/z 415 (M+H)

### Example 79

### 2-[3-(2,2-Diphenyl-vinyloxy)-propyl]-8-methoxy-1,2,3,4-tetrahydro-isoquinoline

### 1-(3-Bromopropyloxy)-2,2-diphenylethene

Dipehenyl-acetaldehyde (Aldrich, D20,425-0) (1 g, 5.1 mmol) was dissolved in CH₂Cl₂ (10 mL) and tetrabutylammonium bromide (Aldrich, 19,311-9) (161 mg, 0.5 mmol) was added followed by 1.2MNaOH solution (10 mL) and 1,3-dibromopropane (Aldrich, 12,590-3) (5.14 g, 25.5 mmol) with vigorous stirring. The reaction was stirred at RT for 18 hrs and acidified with 2M HCl (10 mL). The organic phase was separated and washed well with H₂O, before being dried (MgSO₄). The solvent was removed in vacuo and the residue was purified via flash chromatography eluting with EtOAc/isohexane (3:97) to afford a colourless oil. Yield 890 mg (55%).

### 2-[3-(2,2-Diphenyl-vinyloxy)-propyl]-8-methoxy-1,2,3,4-tetrahydro-isoquinoline

Prepared according to the method described in Example 5. HPLC retention time 5.02 min. 1H NMR (400 MHz CDCl₃)□_{H} 2.0 (2H), 2.65-2.70 (4H), 2.85-2.90 (2H), 3.55 (2H), 3.80 (3H), 4.00-4.05 (2H); 6.55 (1H), 6.65 (1H), 6.70 (1H) 7.10 (1H), 7.18-7.24 (4H), 7.25-7.35 (4H), 7.38-7.44 (2H). Mass spectrum (ES+) m/z 400 (M+H).

### Example 80

### N-Benzhydryl-2-(7-methoxy-1,3,4,5-tetrahydro-benzo[c]azepin-2-yl)-acetamide

Prepared according to the method described in Example 56. HPLC retention time 4.40 min. Mass spectrum (ES+) m/z 401 (M+H).

### Example 81

### N-(2,2-Diphenyl-ethyl)-2-(7-methoxy-1,3,4,5-tetrahydro-benzo[c]azepin-2-yl)-acetamide

Prepared according to the method described in Example 56. HPLC retention time 4.39 min. Mass spectrum (ES+) m/z 415 (M+H).

### Example 82

### N-(3,3-Diphenyl-propyl)-2-(7-methoxy-1,3,4,5-tetrahydro-benzo[c]azepin-2-yl)-acetamide

Prepared according to the method described in Example 56. HPLC retention time 4.47 min. Mass spectrum (ES+) m/z 429 (M+H).

### Example 83

### N,N-Dibenzyl-2-(7-methoxy-1,3,4,5-tetrahydro-benzo[c]azepin-2-yl)-acetamide

Prepared according to the method described in Example 56. HPLC retention time 4.47 min. Mass spectrum (ES+) m/z 415 (M+H).

### Example 84

### 2-Thiophen-2-ylmethyl-1,2,3,4-tetrahydro-isoquinolin-8-ol

A solution of 1,2,3,4-tetrahydro-isoquinolin-8-ol acetic acid salt (75 mg, 0.358 mmol) and Et₃N (36 mg, 0.358 mmol) in CH₃OH (2 mL) was stirred at ambient temperature for 30 mins. 2-Thiophenecarboxaldehyde (Aldrich T3,240-9) (40 mg, 0.358 mmol) was added and the reaction was stirred for 2 hrs at room temperature. Sodium cyanoborohydride (Aldrich, 15,615-9) (23 mg, 0.358 mmol) was added and the reaction was stirred at RT for 18 hrs. The solvent was removed in vacuo and the residue was purified via flash chromatography eluting with MeOH/CH₂Cl₂ (2:98) to afford the title compound as a white solid. Yield 28 mg (32%). HPLC retention time, 3.43 min. 1H NMR (400 MHz (CD₃)₂SO)□_{H} 2.70-2.75 (2H), 2.85-2.90 (2H), 3.60 (2H), 3.95 (2H), 6.50-6.60 (2H), 6.90-6.95 (1H), 6.95-7.0 (1H), 7.05 (1H), 7.35 (1H). Mass spectrum (ES+) m/z 246 (M+H).

### Example 85

### (1H-Benzoimidazol-5-yl)-(3,4-dihydro-1-isoquinolin-2-yl)-methanone

To a solution of 5-benzimidazolecarboxylic acid (Aldrich, 29,678-3) (324 mg, 2 mmol) in CH₂Cl₂/DMF (9:1) (10 mL) was added: 1,2,3,4-tetrahydro-isoquinoline (Aldrich, T1,300-5) (320 mg, 2.4 mmol), Et₃N (404 mg, 4 mmol), 1-hydroxybenzotriazole (Acros, 16916) (405 mg, 3 mmol) and 1-[3-(dimethylamino)-propyl]-3-ethyl-carbodiimide (ACT, RC8102) (460 mg, 2.4 mmol) and the reaction was stirred at RT for 18 hrs. The reaction mixture was diluted with EtOAc (10 mL), washed (5% citric acid), (sat. sodium bicarbonate), and (brine). The organic layer was dried (MgSO₄) and concentrated in vacuo. The residue was purified via flash chromatography eluting with MeOH/CH₂Cl₂ (5:95) to afford the title compound as a brown oil. Yield 15 mg (3%). HPLC retention time 3.09 min. Mass spectrum (ES+) m/z 278 (M+H).

### Example 86

### N-(3,3-Diphenyl-propyl)-2-(7-methoxy-3,4-dihydro-1H-isoquinolin-2-yl)-acetamide

### [2-(4-methoxy-phenyl)-ethyl-carbamic acid methyl ester

4-Methoxyphenethylamine (Aldrich, 18,730-5) (25.8 g, 171 mmol) and Et₃N (20.7 g, 205 mmol) were dissolved in anhydrous THF (1 ltr) and cooled to 0° C. Methyl chloroformate (Aldrich, M3,530-4) 80.8 g, 855 mmol) was added drop wise keeping the temperature at 0° C. After addition the reaction was stirred at 0° C. for a further 2 hrs and at RT for 18 hrs. Deionised H2O (250 mL) was added and the resulting solution was extracted into Et₂O (400 mL) and EtOAc (2×300 mL). The combined extracts were washed with brine (3×500 mL) and 1M HCl (3×400 mL). The organic layer was dried over dried MgSO₄ and the solvent was removed in vacuo to afford a yellow oil which quickly solidified. This was slurried in isohexane, filtered and washed with isohexane to afford [2-(4-methoxy-phenyl)-ethyl]-carbamic acid methyl ester as a white solid, which was used without further purification. Yield 29 g (83%).

### 7-Methoxy-3,4-dihydro-2H-isoquinolin-1-one

Phosphorous pentoxide (Fisher, P/3000/53) (14.2 g, 50 mmol) was added in portions to methanesulphonic acid (Avocado, 13565) (25 mL), and the mixture was heated to 130° C. [2-(4-Methoxy-phenyl)-ethyl]-carbamic acid methyl ester (5.23 g, 25 mmol) was added in portions and the mixture was heated at 140° C. for a further 1 hr. The reaction was allowed to cool to ca. 80° C. and it was carefully added to ice with rapid stirring. This solution was extracted with CH₂Cl₂ (3×50 mL) and the combined extracts were washed with brine (2×50 mL), dried (MgSO₄) and the solvent removed in vacuo. The residue was purified via flash chromatography eluting with MeOH/CH₂Cl₂ (10:90) to afford 7-methoxy-3,4-dihydro-2H-isoquinolin-1-one. Yield 3.3 g (75%). HPLC retention time 3.41 min (Solvent: MeCN/H₂O/0.05% HCOOH, 5-95% gradient H₂O-10 min. Column: Xterra 50×4.60 i.d., C18 reverse phase. Flow rate: 1.51 mL/min.). Mass spectrum (ES+) m/z 178 (M+H).

### 7-Methoxy-1,2,3,4-tetrahydro-isoquinoline hydrochloride

Lithium aluminium hydride, 1.0M solution in THF (Aldrich, 21,277-6) (22 mL, 22 mmol) was added drop wise to 7-methoxy-3,4-dihydro-2H-isoquinolin-1-one (3.0 g, 17 mmol) in THF (25 mL) at RT. After addition the reaction was refluxed for 3 hrs. The reaction was cooled to 0° C. and quenched by the careful addition of deionised H₂O (1 mL), 10% NaOH solution (1 mL) and deionised H₂O (3 mL). The basic suspension was filtered through celite and extracted into EtOAc (3×150 mL). The combined extracts were dried over MgSO₄ and the solvent was removed in vacuo. The residue was purified via flash chromatography eluting with MeOH/CH₂Cl₂ (10:90) to afford 7-methoxy-1,2,3,4-tetrahydro-isoquinoline. This was dissolved in EtOAc (110 mL) and hydrogen chloride, 2.0 m solution in Et₂O (Aldrich, 45,518-0) (10 mL) was added drop wise, which formed a white ppte. The solid was filtered off and washed with Et₂O to afford 7-methoxy-1,2,3,4-tetrahydro-isoquinoline hydrochloride as a white solid. Yield 1.4 g (42%). HPLC retention time, 3.05 min. Mass spectrum (ES+) m/z 164 (M+H).

### N-(3,3-Diphenyl-propyl)-2-(7-methoxy-3,4-dihydro-1H-isoquinolin-2-yl)-acetamide

7-Methoxy-1,2,3,4-tetrahydro-isoquinoline hydrochloride (200 mg, 1 mmol) was stirred in MeCN (10 mL) with K₂CO₃ (276 mg, 2 mmol) and TBAI (Aldrich, 14,077-5) (74 mg, 0.2 mmol) for 30 mins. 2-Chloro-N-(3,3-diphenyl-propyl)-acetamide (288 mg, 1 mmol) was added and the reaction was refluxed for 24 hrs. The reaction was cooled, diluted with MeCN (10 mL) and the insoluble material was removed via filtration. The solvent was removed in vacuo and the residue was purified via flash chromatography eluting with EtoAc/isohexane (1:4) to afford the title compound as an orange oil. Yield 150 mg (36%) HPLC retention time, 4.45 min. Mass spectrum (ES+) m/z 415 (M+H).

### Example 87

### N,N-Dibenzyl-2-(7-methoxy-3,4-dihydro-1H-isoquinolin-2-yl)-acetamide

Prepared according to the method described in Example 86. HPLC retention time 4.53 min. Mass spectrum (ES+) m/z 401 (M+H).

### Example 88

### Dibenzyl-[2-(7-methoxy-3,4-dihydro-1H-isoquinolin-2-yl)-ethyl]-amine

Lithium aluminium hydride, 1.0M solution in THF (Aldrich, 21,277-6) (0.42 mL, 0.42 mmol) was added drop wise to N,N-Dibenzyl-2-(7-methoxy-3,4-dihydro-1H-isoquinolin-2-yl)-acetamide (140 mg, 0.35 mmol). After addition the reaction was refluxed for 3 hrs. The reaction was cooled to 0° C. and quenched by the careful addition of deionised H₂O (1 mL), 10% NaOH solution (1 mL) and deionised H₂O (3 mL). The basic suspension was filtered through celite and extracted into EtOAc (3×150 mL). The combined extracts were dried over MgSO₄ and the solvent was removed in vacuo. The residue was purified via flash chromatography eluting with MeOH/CH₂Cl₂ (10:90) to afford Dibenzyl-[2-(7-methoxy-3,4-dihydro-1H-isoquinolin-2-yl)-ethyl]-amine. HPLC retention time 5.13 min. Mass spectrum (ES+) m/z 387 (M+H).

### Example 89

### (3,3-Diphenyl-propyl)-[2-(7-methoxy-3,4-dihydro-1H-isoquinolin-2-yl)-ethyl]amine

Prepared according to the method described in Example 88. HPLC retention time, 4.91 min. Mass spectrum (ES+) m/z 401 (M+H).

### Example 90

### 2-(3,5-Bis-trifluoromethyl-benzyl)-1,2,3,4-tetrahydro-isoquinolin-6-ol

A solution of 1,2,3,4-Tetrahydro-isoquinolin-6-ol (0.05 g, 0.13 mmol), 1-bromomethyl-3,5-bis-trifluoromethyl-benzene (0.041 g, 0.13 mmol) and K₂CO₃ (0.018 g, 0.13 mmol) in MeCN (2 mL) was shaken at ambient temperature for 16 hours. The reaction was filtered through a plug of cotton wool, concentrated in vacuo and purified by flash chromatography to afford the title compound. HPLC retention time, 1.26 min. Mass spectrum (ES+) m/z 376 (M+H).

### Example 91

### 2-(2-Chloro-6-fluoro-benzyl)-1,2,3,4-tetrahydro-isoquinolin-8-ol

Prepared according to the method described in Example 90. HPLC retention time 0.97 min. Mass spectrum (ES+) m/z 292 (M+H).

### Example 92

### 2-(2,5-Difluoro-benzyl)-1,2,3,4-tetrahydro-isoquinolin-8-ol

Prepared according to the method described in Example 90. HPLC retention time, 1.26 min. Mass spectrum (ES+) m/z 276 (M+H).

### Example 93

### 2-(3,5-Difluoro-benzyl)-1,2,3,4-tetrahydro-isoquinolin-8-ol

Prepared according to the method described in Example 90. HPLC retention time 0.97 min. Mass spectrum (ES+) m/z 276 (M+H).

### Example 94

### 2-(4-Trifluoromethylsulfanyl-benzyl)-1,2,3,4-tetrahydro-isoquinolin-8-ol

Prepared according to the method described in Example 90. HPLC retention time 1.24 min. Mass spectrum (ES+) m/z 340 (M+H);

### Example 95

### 2-(3,5-Bis-trifluoromethyl-benzyl)-1,2,3,4-tetrahydro-isoquinolin-8-ol

Prepared according to the method described in Example 90. HPLC retention time, 1.27 min. Mass spectrum (ES+) m/z 376 (M+H).

### Example 96

### 2-[4,4-Bis-(4-fluoro-phenyl)-butyl]-1,2,3,4-tetrahydro-isoquinolin-8-ol

Prepared according to the method described in Example 90. HPLC retention time 1.46 min. Mass spectrum (ES+) m/z 394 (M+H).

### Example 97

### 2-[4,4-Bis-(4-hydroxy-3,5-dimethyl-phenyl)-pentyl]-1,2,3,4-tetrahydroisoquinolin-8-ol

Prepared according to the method described in Example 90. HPLC retention time 1.41 min. Mass spectrum (ES+) m/z 460 (M+H).

### Example 98

### N,N-Dibenzyl-2-(8-ethoxy-3,4-dihydro-1H-isoquinolin-2-yl)acetamide

Prepared according to the method described in Example 14. HPLC retention time 4.72 min. Mass Spectrum (ES+) m/z 415 (M+H).

### Example 99

### N-(4,4-Diphenyl-butyl)-2-(8-ethoxy-3,4-dihydro-1H-isoquinolin-2-yl)acetamide

Prepared according to the method described in Example 14: HPLC retention time 4.68 min. Mass Spectrum (ES+) m/z 443(M+H).

### Example 100

### 2-(8-Ethoxy-3,4-dihydro-1H-isoquinolin-2-yl)-1-(3phenyl-2,3-dihydro-benzo[1,4]oxazin-4-yl)-ethanone

Prepared according to the method described in Example 56. HPLC retention time 4.60 min. Mass Spectrum (ES+) m/z 429(M+H).

### Example 101

### N-(3-Benzhydryloxy-propyl)-2-(8-methoxy-3,4-dihydro-1H-isoquinolin-2-yl)acetamide

Prepared according to the method described in Example 14. HPLC retention time 4.43 min. Mass Spectrum (ES+) m/z 445 (M+H).

### Example 102

### 2-(1,3-Dihydro-isoindol-2-yl)-N-(3,3-diphenyl-propyl)acetamide

Prepared according to the method described in Example 40. HPLC retention time 4.33 min. Mass Spectrum (ES+) m/z 371(M+H).

### Example 103

### N-(2-Benzhydrylsulphanyl-ethyl)-2-(8-methoxy-3,4-dihydro-1H-isoquinolin-2-yl)acetamide

Prepared according to the method described in Example 14. HPLC retention time 4.55 min. Mass Spectrum (ES+) m/z 447(M+H).

### Example 104

### 2-(8-Allyloxy-3,4-dihydro-1H-isoquinolin-2-yl)-N-(3,3-diphenyl-propyl)acetamide

Prepared according to the method described in Example 14. HPLC retention time 4.59 min. Mass Spectrum (ES+) m/z 441(M+H).

### Example 105

### 2-(4-Amino-1,3-dihydro-isoindol-2-yl)-N-(2,2-diphenyl-ethyl)acetamide

Prepared according to the method described in Example 14. HPLC retention time 3.93 min. Mass Spectrum (ES+) m/z 373 (M+H).

### Example 106

### 2-(4-Amino-1,3-dihydro-isoindol-2-yl)-N-(3,3-diphenyl-propyl)acetamide

Prepared according to the method described in Example 14. HPLC retention time 4.02 min. Mass Spectrum (ES+) m/z 386(M+H).

### Example 107

### 2-(4-Amino-1,3-dihydro-isoindol-2-yl)-N-(4,4-diphenyl-butyl)acetamide

Prepared according to the method described in Example 14. HPLC retention time 4.14 min. Mass Spectrum (ES+) m/z 400(M+H).

### Example 108

### 2-(4-Amino-1,3-dihydro-isoindol-2-yl)-N,N-dibenzylacetamide

Prepared according to the method described in Example 14. HPLC retention time 4.03 min. Mass Spectrum (ES+) m/z 372(M+H).

### Example 109

### 2-[4,4-Bis-(4-fluorophenyl)-butyl]-2,3-dihydro-1H-isoindol-4-ylamine

Prepared according to the method described in Example 14. HPLC retention time 4.50 min. Mass Spectrum (ES+) m/z 379(M+H).

### Example 110

### N-[2-(Diphenylmethanesulphinyl)-ethyl]-2-(8-methoxy-3,4-dihydro-1H-isoquinolin-2-yl)acetamide

N-[2-(Diphenylmethanesulphinyl)-ethyl]-2-(8-methoxy-3,4-dihydro-1H-isoquinolin-2-yl)acetamide was prepared from N-(2-benzhydrylsulphanyl-ethyl)-2-(8-methoxy-3,4-dihydro-1H-isoquinolin-2-yl)acetamide (1 eq) and mCPBA (1 eq) in CH₂Cl₂ to afford the title compound. HPLC retention time 3.85 min. Mass Spectrum (ES+) m/z 463(M+H).

### Example 111

### N-[2-(Diphenylmethanesulphonyl)-ethyl]-2-(8-methoxy-3,4-dihydro-1H-isoquinolin-2-yl)acetamide

N-[2-(Diphenylmethanesulphonyl)-ethyl]-2-(8-methoxy-3,4-dihydro-1H-isoquinolin-2-yl)acetamide was prepared from N-(2-benzhydrylsulphanyl-ethyl)-2-(8-methoxy-3,4-dihydro-1H-isoquinolin-2-yl)acetamide (1 eq) and mCPBA (2 eq) in CH₂Cl₂ to afford the title compound. HPLC retention time 3.26 min. Mass Spectrum (ES+) m/z 479(M+H).

### Example 112

### 2-(8-Methoxy-3,4-dihydro-1H-isoquinolin-2-yl)-N-(1phenyl-ethyl)acetamide

Prepared according to the method described in Example 14. HPLC retention time 4.04 min. Mass Spectrum (ES+) m/z 325(M+H).

### Example 113

### 2-(3,4-Dihydro-1H-isoquinolin-2-yl)-N-(1phenyl-ethyl)acetamide

Prepared according to the method described in Example 14. HPLC retention time 3.99 min. Mass Spectrum (ES+) m/z 295(M+H).

### Example 114

### 2-(Benzhydryl-amino)-1-(1,3-dihydro-isoindol-2-yl)-ethanone

Prepared according to the method described in Example 14. HPLC retention time 4.12 min. Mass Spectrum (ES+) m/z 343(M+H).

### Example 115

### 2-(8-Amino-3,4-dihydro-1H-isoquinolin-2-yl)-N-benzhydrylacetamide

Prepared according to the method described in Example 14. HPLC retention time 3.99 min. Mass Spectrum (ES+) m/z 372(M+H).

### Example 116

### 2-(8-Amino-3,4-dihydro-1H-isoquinolin-2-yl)-N-(4,4-diphenyl-butyl)acetamide

Prepared according to the method described in Example 14. HPLC retention time 4.13 min. Mass Spectrum (ES+) m/z 414(M+H).

### Example 117

### 2-[4,4-Bis-(4-fluorophenyl)-butyl]-1,2,3,4-tetrahydro-isoquinolin-8-ylamine

Prepared according to the method described in Example 14. HPLC retention time 4.60 min. Mass Spectrum (ES+) m/z 393(M+H).

### Example 118

### 2-(8-Amino-3,4-dihydro-1H-isoquinolin-2-yl)-N-(2,2-diphenyl-ethyl)acetamide

Prepared according to the method described in Example 14. HPLC retention time 4.11 min. Mass Spectrum (ES+) m/z 386(M+H)

### Example 119

### 2-(8-Acetylamino-3,4-dihydro-1H-isoquinolin-2-yl)-N-(4,4-diphenyl-butyl)acetamide

2-(8-Acetylamino-3,4-dihydro-1H-isoquinolin-2-yl)-N-(4,4-diphenyl-butyl)acetamide was prepared from 2-(8-amino-3,4-dihydro-1H-isoquinolin-2-yl)-N-(4,4-diphenyl-butyl)acetamide (1 eq.) and acetylchloride (1 eq) in CH₂Cl₂ to afford the title compound. HPLC retention time 4.21 min. Mass Spectrum (ES+) m/z 456(M+H).

### Example 120

### N-[3,3-Bis-(4-methoxyphenyl)-propyl]-2-(1,3-dihydro-isoindol-2-yl)acetamide

Prepared according to the method-described in Example 14. HPLC retention time 4.30 min. Mass Spectrum (ES+) m/z 431(M+H).

### Example 121

### N-[3,3-Bis-(4-methoxyphenyl)-propyl]-2-(3,4-dihydro-1H-isoquinolin-2-yl)acetamide

Prepared according to the method described in Example 14. HPLC retention time 4.42 min. Mass Spectrum (ES+) m/z 445(M+H).

### Example 122

### N-[3,3-Bis-(4-methoxyphenyl)-propyl]-2-(8-methoxy-3,4-dihydro-1H-isoquinolin-2-yl)acetamide

Prepared according to the method described in Example 14. HPLC retention time 4.31 min. Mass Spectrum (ES+) m/z 475(M+H).

### Example 123

### N-[3-(3,4-Bis-acetylaminophenyl)-3phenyl-propyl]-2-(3,4-dihydro-1H-isoquinolin-2-yl)acetamide

Prepared according to the method described in Example 14. HPLC retention time 3.67 min. Mass Spectrum (ES+) m/z 499(M+H).

### Example 124

### N-(4,4-Diphenyl-butyl)-2-(8-methanesulphonylamino-3,4-dihydro-1H-isoquinolin-2-yl)acetamide

N-(4,4-Diphenyl-butyl)-2-(8-methanesulphonylamino-3,4-dihydro-1H-isoquinolin-2-yl)acetamide was prepared from 2-(8-amino-3,4-dihydro-1H-isoquinolin-2-yl)-N-(4,4-diphenyl-butyl)acetamide (1 eq), methanesulphonylchloride (1 eq) and triethylamine (1 eq) in CH₂Cl₂ to afford the title compound. HPLC retention time 3.99 min. Mass Spectrum (ES+) m/z 492(M+H).

### Example 125

### N-[Bis-(4-fluorophenyl)methyl]-2-(1,3-dihydro-isoindol-2-yl)acetamide

Prepared according to the method described in Example 14. HPLC retention time 4.28 min. Mass Spectrum (ES+) m/z 379(M+H).

### Example 126

### N-[Bis-(4-fluorophenyl)methyl]-2-(3,4-dihydro-1H-isoquinolin-2-yl)acetamide

Prepared according to the method described in Example 14. HPLC retention time 4.42 min. Mass Spectrum (ES+) m/z 393(M+H).

### Example 127

### N-[Bis-(4-fluorophenyl)methyl]-2-(8-methoxy-3,4-dihydro-1H-isoquinolin-2-yl)acetamide

Prepared according to the method described in Example 14. HPLC retention time 4.44 min. Mass Spectrum (ES+) m/z 423(M+H).

### Example 128

### N-[Bis-(4-fluorophenyl)methyl]-2-(6,7-dimethoxy-3,4-dihydro-1H-isoquinolin-2-yl)acetamide

Prepared according to the method described in Example 14. HPLC retention time 4.13 min. Mass Spectrum (ES+) m/z 453 (M+H).

### Example 129

### 3-(5-Amino-3,4-dihydro-1H-isoquinolin-2-yl)-N-(3,3-diphenyl-propyl)propionamide

Prepared according to the method described in Example 14. HPLC retention time 3.87 min. Mass Spectrum (ES+) m/z 414(M+H).

### Example 130

### 2-(5-Amino-3,4-dihydro-1H-isoquinolin-2-yl)-N-(2,2-diphenyl-ethyl)acetamide

Prepared according to the method described in Example 14. HPLC retention time 3.90 min. Mass Spectrum (ES+) m/z 386(M+H).

### Example 131

### 2-(Dimethoxy-3,4-dihydro-1H-isoquinolin-2-yl)-N-(4,4-diphenyl-butyl)acetamide

Prepared according to the method described in Example 14. HPLC retention time 4.25 min. Mass Spectrum (ES+) m/z 459(M+H).

### Example 132

### 3-(6,7-Dimethoxy-3,4-dihydro-1H-isoquinolin-2-yl)-N-(3,3-diphenyl-propyl)propionamide

Prepared according to the method described in Example 14. HPLC retention time 4.01 min. Mass Spectrum (ES+) m/z 459(M+H).

### Example 133

### 2-(6,7-Dimethoxy-3,4-dihydro-1H-isoquinolin-2-yl)-N-(2,2-diphenyl-ethyl)acetamide

Prepared according to the method described in Example 14: HPLC retention time 4.07 min. Mass Spectrum (ES+) m/z 431(M+H).

### Example 134

### 3-(8-Amino-3,4-dihydro-1H-isoquinolin-2-yl)-N-(3,3-diphenyl-propyl)propionamide

Prepared according to the method described in Example 14. HPLC retention time 3.97 min. Mass Spectrum (ES+) m/z 414(M+H).

### Example 135

### N-(3-Carbazol-9-yl-propyl)-2-(8-methoxy-3,4-dihydro-1H-isoquinolin-2-yl)acetamide

Prepared according to the method described in Example 14. HPLC retention time 4.47 min. Mass Spectrum (ES+) m/z 428(M+H).

### Example 136

### N-(3-Carbazol-9-yl-propyl)-2-(8-hydroxy-3,4-dihydro-1H-isoquinolin-2-yl)acetamide

Prepared according to the method described in Example 14. HPLC retention time 4.07 min. Mass Spectrum (ES+) m/z 414(M+H).

### Example 137

### N-[3-(5-Chloro-2-methyl-indol-1-yl)-propyl]-2-(8-methoxy-3,4-dihydro-1H-isoquinolin-2-yl)acetamide

Prepared according to the method described in Example 14. HPLC retention time 4.48 min. Mass Spectrum (ES+) m/z 426(M+H).

### Example 138

### N-[3-(5-Chloro-2-methyl-indol-1-yl)-propyl]-2-(8-hydroxy-3,4-dihydro-1H-isoquinolin-2-yl)acetamide

Prepared according to the method described in Example 14. HPLC retention time 4.06 min. Mass Spectrum (ES+) m/z 413(M+H).

### Example 139

### 1-Benzhydryl-3-[2-(8-methoxy-3,4-dihydro-1H-isoquinolin-2-yl)-ethyl]-thiourea

1-Benzhydryl-3-[2-(8-methoxy-3,4-dihydro-1H-isoquinolin-2-yl)-ethyl]-thiourea was prepared from 2-(8-methoxy-3,4-dihydro-1H-isoquinolin-2-yl)ethylamine (1 eq) and benzhydryl isothiocyanate (1 eq) in CH₂Cl₂ to afford the title compound. HPLC retention time 4.55 min. Mass Spectrum (ES+) m/z 432(M+H).

### Example 140

### 1-Benzhydryl-3-[2-(6,7-dimethoxy-3,4-dihydro-1H-isoquinolin-2-yl)-ethyl]-thiourea

Prepared according to the method described in Example 139. HPLC retention time 4.23 min. Mass Spectrum (ES+) m/z 462(M+H).

### Example 141

### 1-Benzhydryl-3-[2-(8-methoxy-3,4-dihydro-1H-isoquinolin-2-yl)-ethyl]-urea

Prepared according to the method described in Example 139. HPLC retention time 4.18 min. Mass Spectrum (ES+) m/z 416(M+H).

### Example 142

### 1-Benzhydryl-3-[2-(6,7-dimethoxy-3,4-dihydro-1H-isoquinolin-2-yl)-ethyl]-urea

Prepared according to the method described in Example 139. HPLC retention time 3.86 min. Mass Spectrum (ES+) m/z 446(M+H).

### Example 143

### 1-(2,2-Diphenyl-ethyl)-3-[2-(8-methoxy-3,4-dihydro-1H-isoquinolin-2-yl)-ethyl]-thiourea

Prepared according to the method described in Example 139. HPLC retention time 4.55 min. Mass Spectrum (ES+) m/z 446(M+H).

### Example 144

### 1-[2-(6,7-Dimethoxy-3,4-dihydro-1H-isoquinolin-2-yl)-ethyl]-3-(2,2-diphenyl-ethyl)-thiourea

Prepared according to the method described in Example 139. HPLC retention time 4.23 min. Mass Spectrum (ES+) m/z 476(M+H).

### Example 145

### 2-(8-Hydroxy-3,4-dihydro-1H-isoquinolin-2-yl)-1-phenothiazin-10-yl-ethanone

Prepared according to the method described in Example 56. HPLC retention time 4.06 min. Mass Spectrum (ES+) m/z 389(M+H).

### Example 146

### 2-(8-Methoxy-3,4-dihydro-1H-isoquinolin-2-yl)-1-phenothiazin-10-yl-ethanone

Prepared according to the method described in Example 56. HPLC retention time 4.56 min. Mass Spectrum (ES+) m/z 403(M+H).

### Example 147

### 1-(2-Chloro-phenothiazin-10-yl)-2-(8-hydroxy-3,4-dihydro-1H-isoquinolin-2-yl)-ethanone

Prepared according to the method described in Example 56. HPLC retention time 4.33 min. Mass Spectrum (ES+) m/z 4.23(M+H).

### Example 148

### 1-(2-Chloro-phenothiazin-10-yl)-2-(8-methoxy-3,4-dihydro-1H-isoquinolin-2-yl)-ethanone

Prepared according to the method described in Example 56. HPLC retention time 4.82 min. Mass Spectrum (ES+) m/z 438(M+H).

### Example 149

### 2-(8-Methoxy-3,4-dihydro-1H-isoquinolin-2-yl)-1-(5-oxo-5H-5lambda*4*-phenothiazin-10-yl)-ethanone

Prepared according to the method described in Example 110. HPLC retention time 3.94 min. Mass Spectrum (ES+) m/z 419(M+H).

### Example 150

### 2-(8-Hydroxy-3,4-dihydro-1H-isoquinolin-2-yl)-1-phenoxazin-10-yl-ethanone

Prepared according to the method described in Example 56. HPLC retention time 4.12 min. Mass Spectrum (ES+) m/z 373(M+H).

### Example 151

### 2-(8-Hydroxy-3,4-dihydro-1H-isoquinolin-2-yl)-1-(2-trifluoromethyl-phenothiazin-10-yl)-ethanone

Prepared according to the method described in Example 56. HPLC retention time 4.40 min. Mass Spectrum (ES+) m/z 457(M+H).

### Example 152

### 2-(8-Methoxy-3,4-dihydro-1H-isoquinolin-2-yl)-1-(2-trifluoromethyl-phenothiazin-10-yl)-ethanone

Prepared according to the method described in Example 56. HPLC retention time 4.83 min. Mass Spectrum (ES+) m/z 471 (M+H).

### Example 153

### 1-(2-Acetyl-phenothiazin-10-yl)-2-(8-hydroxy-3,4-dihydro-1H-isoquinolin-2-yl)-ethanone

Prepared according to the method described in Example 56. HPLC retention time 3.93 min. Mass Spectrum (ES+) m/z 431 (M+H).

### Example 154

### 1-(2-Acetyl-phenothiazin-10-yl)-2-(8-methoxy-3,4-dihydro-1H-isoquinolin-2-yl)-ethanone

Prepared according to the method described in Example 56. HPLC retention time 4.37 min. Mass Spectrum (ES+) m/z 445(M+H).

### Example 155

### 2-(8-Hydroxy-3,4-dihydro-1H-isoquinolin-2-yl)-N,N-diphenylacetamide

Prepared according to the method-described in Example 56. HPLC retention time 3.75 min. Mass Spectrum (ES+) m/z 359(M+H).

### Example 156

### 2-(8-Methoxy-3,4-dihydro-1H-isoquinolin-2-yl)-N,N-diphenylacetamide

Prepared according to the method described in Example 56. HPLC retention time 4.22 min. Mass Spectrum (ES+) m/z 373(M+H).

### Example 157

### 2-(6,7-Dimethoxy-3,4-dihydro-1H-isoquinolin-2-yl)-1-(5,5-dioxo-5H-5lambda*6*-phenothiazin-10-yl)-ethanone

Prepared according to the method described in Example 111. HPLC retention time 3.76 min. Mass Spectrum (ES+) m/z 465(M+H).

### Example 158

### 2-(6,7-Dimethoxy-3,4-dihydro-1H-isoquinolin-2-yl)-1-phenothiazin-10-yl-ethanone

Prepared according to the method described in Example 56. HPLC retention time 4.19 min. Mass Spectrum (ES+) m/z 433(M+H).

### Example 159

### 1-(2-Chloro-phenothiazin-10-yl)-2-(6,7-dimethoxy-3,4-dihydro-1]-isoquinolin-2-yl)-ethanone

Prepared according to the method described in Example 56. HPLC retention time 4.46 min. Mass Spectrum (ES+) m/z 468(M+H).

### Example 160

### 2-(6,7-Dimethoxy-3,4-dihydro-1H-isoquinolin-2-yl)-1-(2-trifluoromethyl-phenothiazin-10-yl)-ethanone

Prepared according to the method described in Example 56. HPLC retention time 4.53 min. Mass Spectrum (ES+) m/z 501(M+H).

### Example 161

### 1-(2-Acetyl-phenothiazin-10-yl)-2-(6,7-dimethoxy-3,4-dihydro-1H-isoquinolin-2-yl)-ethanone

Prepared according to the method described in Example 56. HPLC retention time 4.06 min. Mass Spectrum (ES+) m/z 475(M+H).

### Example 162

### 2-(6,7-Dimethoxy-3,4-dihydro-1H-isoquinolin-2-yl)-1-(2-methylsulphanyl-phenothiazin-10-yl)-ethanone

Prepared according to the method described in Example 56. HPLC retention time 4.40 min. Mass Spectrum (ES+) m/z 479(M+H).

### Example 163

### 2-(6,7-Dimethoxy-3,4-dihydro-1H-isoquinolin-2-yl)-1-(5-oxo-5H-5lambda*4*-phenothiazin-10-yl)-ethanone

Prepared according to the method described in Example 110. HPLC retention time 3.56 min. Mass Spectrum (ES+) m/z 449(M+H).

### Example 164

### 2-(8-Hydroxy-3,4-dihydro-1H-isoquinolin-2-yl)-1-(2-methylsulphanyl-phenothiazin-10-yl)-ethanone

Prepared according to the method described in Example 56. HPLC retention time 4.26 min. Mass Spectrum (ES+) m/z 435(M+H).

### Example 165

### 2-(8-Methoxy-3,4-dihydro-1H-isoquinolin-2-yl)-1-(2-methylsulphanyl-phenothiazin-10-yl)-ethanone

Prepared according to the method described in Example 56. HPLC retention time 4.70 min. Mass Spectrum (ES+) m/z 449(M+H).

### Example 166

### Phenothiazine-10-carboxylic acid [2-(8-hydroxy-3,4-dihydro-1H-isoquinolin-2-yl)-ethyl]-amide

Prepared according to the method described in Example 56 with the following modification: tetrabutylammonium iodide was not used and triethylamine was used as a base. HPLC retention time 3.86 min. Mass Spectrum (ES+) m/z 418(M+H).

### Example 167

### Phenothiazine-10-carboxylic acid [2-(6,7-dimethoxy-3,4-dihydro-1H-isoquinolin-2-yl)-ethyl]-amide

Prepared according to the method described in Example 56 with the following modification: tetrabutylammonium iodide was not used and triethylamine was used as a base. HPLC retention time 4.04 min. Mass Spectrum (ES+) m/z 357(M+H).

### Example 168

### Phenothiazine-10-carboxylic acid [2-(8-methoxy-3,4-dihydro-1H-isoquinolin-2-yl)-ethyl]-amide

Prepared according to the method described in Example 56 with the following modification: tetrabutylammonium iodide was not used and triethylamine was used as a base. HPLC retention time 4.62 min. Mass Spectrum (ES+) m/z 357(M+H).

### Example 169

### Phenoxazine-10-carboxylic acid [2-(8-hydroxy-3,4-dihydro-1H-isoquinolin-2-yl)-ethyl]-amide

Prepared according to the method described in Example 56 with the following modification: tetrabutylammonium iodide was not used and triethylamine was used as a base. HPLC retention time 4.61 min. Mass Spectrum (ES+) m/z 432(M+H).

### Example 170

### Phenoxazine-10-carboxylic acid [2-(8-methoxy-3,4-dihydro-1H-isoquinolin-2-yl)-ethyl]-amide

Prepared according to the method described in Example 56 with the following modification: tetrabutylammonium iodide was not used and triethylamine was used as a base. HPLC retention time 3.84 min. Mass Spectrum (ES+) m/z 402(M+H).

### Example 172

### Phenoxazine-10-carboxylic acid [2-(6,7-dimethoxy-3,4-dihydro-1H-isoquinolin-2-yl)-ethyl]-amide

Prepared according to the method described in Example 56 with the following modification: tetrabutylammonium iodide was not used and triethylamine was used as a base. HPLC retention time 4.26 min. Mass Spectrum (ES+) m/z 416(M+H).

### Example 173

### N-[3,3-Bis-(4-fluorophenyl)-propyl]-3-(8-methoxy-3,4-dihydro-1H-isoquinolin-2-yl)propionamide

Prepared according to the method described in Example 56. HPLC retention time 3.9 min. Mass Spectrum (ES+) m/z 465(M+H).

### Example 174

### (8-Hydroxy-3,4-dihydro-1H-isoquinolin-2-yl)-acetic acid N′,N′-diphenyl-hydrazide

Prepared according to the method described in Example 56. HPLC retention time 3.42 min. Mass Spectrum (ES+) m/z 374(M+H).

### Example 175

### (8-Methoxy-3,4-dihydro-1H-isoquinolin-2-yl)-acetic acid N′,N′-diphenyl-hydrazide

Prepared according to the method described in Example 56. HPLC retention time 3.85 min. Mass Spectrum (ES+) m/z 388(M+H).

### Example 176

### (6,7-Dimethoxy-3,4-dihydro-1H-isoquinolin-2-yl)-acetic acid N′,N′-diphenyl-hydrazide

Prepared according to the method described in Example 56. HPLC retention time 3.55 min. Mass Spectrum (ES+) m/z 418(M+H).

### Example 177

### 4-[2-(8-Hydroxy-3,4-dihydro-1H-isoquinolin-2-yl)-acetyl]-3,4-dihydro-2H-benzo[1,4]oxazine-2-carboxylic acid ethyl ester

Prepared according tote method described in Example 56. HPLC retention time 4.01 min. Mass Spectrum (ES+) m/z 397(M+H).

### Example 178

### 4-[2-(8-Methoxy-3,4-dihydro-1H-isoquinolin-2-yl)-acetyl]-3,4-dihydro-2H-benzo[1,4]oxazine-2-carboxylic acid ethyl ester

Prepared according to the method described in Example 56. HPLC retention time 4.22 min. Mass Spectrum (ES+) m/z 411(M+H).

### Example 179

### 2-(8-Methoxy-3,4-dihydro-1H-isoquinolin-2-yl)-N-(4-phenoxyphenyl)acetamide

Prepared according to the method described in Example 56. HPLC retention time 4.18 min. Mass Spectrum (ES+) m/z 389(M+H).

### Example 180

### 2-(5,8-Dihydro-6H-[1,7]naphthyridin-7-yl)-1-phenoxazin-10-yl-ethanone

Prepared according to the method described in Example 56 with the following modification: triethylamine was used as base. HPLC retention time 3.1 min. Mass Spectrum (ES+) m/z 358(M+H).

### Example 181

### 1-[2-(8-Methoxy-3,4-dihydro-1H-isoquinolin-2-yl)-ethyl]-3-(4-phenoxyphenyl)-urea

Prepared according to the method described in Example 139. HPLC retention time 3.5 min. Mass Spectrum (ES+) m/z 418(M+H).

### Example 182

### 2-(8-Amino-3,4-dihydro-1H-isoquinolin-2-yl)-1-phenoxazin-10-yl-ethanone

Prepared according to the method described in Example 56. HPLC retention time 4.09 min. Mass Spectrum (ES+) m/z 372(M+H).

### Example 183

### 2-(8-Hydroxy-3,4-dihydro-1H-isoquinolin-2-yl)-N-(4-hydroxyphenyl)-Nphenylacetamide

Prepared according to the method described in Example 56. HPLC retention time 3.98 min. Mass Spectrum (ES+) m/z 375(M+H).

### Example 184

### N-(4-Hydroxyphenyl)-2-(8-methoxy-3,4-dihydro-1H-isoquinolin-2-yl)-Nphenylacetamide

Prepared according to the method described in Example 56. HPLC retention time 4.54 min. Mass Spectrum (ES+) m/z 433(M+H).

### Example 185

### 2-(1,3-Dihydro-isoindol-2-yl)-1-phenoxazin-10-yl-ethanone

Prepared according to the method described in Example 56. HPLC retention time 4.44 min. Mass Spectrum (ES+) m/z 343(M+H).

### Example 186

### 2-(8-Hydroxy-3,4-dihydro-1H-isoquinolin-2-yl)-N-(9H-xanthen-9-yl)acetamide

Prepared according to the method described in Example 56. HPLC retention time 3.96 min. Mass Spectrum (ES+) m/z 387(M+H).

### Example 187

### 2-(8-Methoxy-3,4-dihydro-1H-isoquinolin-2-yl)-N-(9H-xanthen-9-yl)acetamide

Prepared according to the method described in Example 56. HPLC retention time 4.56 min. Mass Spectrum (ES+) m/z 401(M+H).

### Example 188

### 2-(5,8-Dihydro-6H-[1,7]naphthyridin-7-yl)-N,N-diphenylacetamide

Prepared according to the method described in Example 56 with the following modification:—triethylamine was used as base. HPLC retention time 3.44 min. Mass Spectrum (ES+) m/z 344(M+H).

### Example 189

### 2-(8-Methoxy-3,4-dihydro-1H-isoquinolin-2-yl)-N,N-bis-(4-methoxyphenyl)acetamide

Prepared according to the method described in Example 56. HPLC retention time 4.22 min. Mass Spectrum (ES+) m/z 433(M+H).

### Example 190

### 2-(8-Hydroxy-3,4-dihydro-1H-isoquinolin-2-yl)-N,N-bis-(4-methoxyphenyl)acetamide

Prepared according to the method described in Example 56. HPLC retention time 3.63 min. Mass Spectrum (ES+) m/z 419(M+H).

### Example 191

### 2-(8-Methoxy-3,4-dihydro-1H-isoquinolin-2-yl)-N-(2-phenoxyphenyl)acetamide

Prepared according to the method described in Example 56. HPLC retention time 4.77 min. Mass Spectrum (ES+) m/z 389(M+H).

### Example 192

### 2-(8-Hydroxy-3,4-dihydro-1H-isoquinolin-2-yl)-N-(2-phenoxyphenyl)acetamide

Prepared according to the method described in Example 56. HPLC retention time 4.17 min. Mass Spectrum (ES+) m/z 375(M+H).

### Example 193

### 1-[(8-Methoxy-3,4-dihydro-1H-isoquinolin-2-yl)-acetyl]-4,4-diphenylsemicarbazide

Prepared according to the method described in Example 56. HPLC retention time 3.76 min. Mass Spectrum (ES+) m/z 431(M+H).

### Example 194

### 2-(8-Methoxy-3,4-dihydro-1H-isoquinolin-2-yl)-1-[2-(5-methyl-[1,3,4]oxadiazol-2-yl)-2,3-dihydro-benzo[1,4]oxazin-4-yl]-ethanone

Prepared according to the method described in Example 56. HPLC retention time 3.76 min. Mass Spectrum (ES+) m/z 431(M+H).

### Example 195

### N-(3-Amino-pyridin-2-yl)-N-(2-hydroxyphenyl)-2-(8-methoxy-3,4-dihydro-1H-isoquinolin-2-yl)acetamide

Prepared according to the method described in Example 56. HPLC retention time 3.79 min. Mass Spectrum (ES+) m/z 405(M+H).

### Example 196

### 3-(8-Methoxy-3,4-dihydro-1H-isoquinolin-2-yl)-1-phenoxazin-10-yl-propan-1-one

Prepared according to the method described in Example 56. HPLC retention time 4.52 min. Mass Spectrum (ES+) m/z 401(M+H).

### Example 197

### 3-(8-Hydroxy-3,4-dihydro-1H-isoquinolin-2-yl)-1-phenoxazin-10-yl-propan-1-one

Prepared according to the method described in Example 56. HPLC retention time 3.93 min. Mass Spectrum (ES+) m/z 387(M+H).

### Example 198

### Methanesulphonic acid 2-(2-oxo-2-phenoxazin-10-yl-ethyl)-1,2,3,4-tetrahydro-isoquinolin-8-yl ester

Prepared according to the method described in Example 124. HPLC retention time 4.23 min. Mass Spectrum (ES+) m/z 452(M+H).

### Example 199

### 1-(2,3-Dihydro-benzo[1,4]oxazin-4-yl)-2-(8-methoxy-3,4-dihydro-1H-isoquinolin-2-yl)-ethanone

Prepared according to the method described in Example 56. HPLC retention time 4.07 min. Mass Spectrum (ES+) m/z 339(M+H).

### Example 200

### 2-(7-Hydroxy-3,4-dihydro-1H-isoquinolin-2-yl)-1-phenoxazin-10-yl-ethanone

Prepared according to the method described in Example 56. HPLC retention time 3.88 min. Mass Spectrum (ES+) m/z 373(M+H).

### Example 201

### 2-(6-Hydroxy-3,4-dihydro-1H-isoquinolin-2-yl)-1-phenoxazin-10-yl-ethanone

Prepared according to the method described in Example 56. HPLC retention time 3.83 min. Mass Spectrum (ES+) m/z 373(M+H).

### Example 202

### 2-(5-Hydroxy-3,4-dihydro-1H-isoquinolin-2-yl)-1-phenoxazin-10-yl-ethanone

Prepared according to the method described in Example 56. HPLC retention time 3.89 min. Mass Spectrum (ES+) m/z 373(M+H).

### Example 203

### 2-(4-Methoxy-1,3-dihydro-isoindol-2-yl)-1-phenoxazin-10-yl-ethanone

Prepared according to the method described in Example 56. HPLC retention time 4.36 min. Mass Spectrum (ES+) m/z 373(M+H).

### Example 204

### N-Methanesulphonyl-N-[2-(2-oxo-2-phenoxazin-10-yl-ethyl)-1,2,3,4-tetrahydro-isoquinolin-8-yl]-methanesulphonamide

Prepared according to the method described in Example 124. HPLC retention time 4.04 min. Mass Spectrum (ES+) m/z 528(M+H).

### Example 205

### N-[2-(2-Oxo-2-phenoxazin-10-yl-ethyl)-1,2,3,4-tetrahydro-isoquinolin-8-yl]-methanesulphonamide

Prepared according to the method described in Example 124. HPLC retention time 2.95 min. Mass Spectrum (ES+) m/z 450(M+H).

### Example 206

### 2-(8-Methoxy-3,4-dihydro-1H-isoquinolin-2-yl)-1-(1-methyl-1H-4-oxa-1,2,9-triaza-cyclopenta[b]naphthalen-9-yl)-ethanone

Prepared according to the method described in Example 56. HPLC retention time 4.11 min. Mass Spectrum (ES+) m/z 391(M+H).

### Example 207

### 2-(8-Methoxy-3,4-dihydro-1H-isoquinolin-2-yl)-1-phenoxazin-10-yl-propan-1-one

Prepared according to the method described in Example 56. HPLC retention time 4.98 min. Mass Spectrum (ES+) m/z 401(M+H).

### Example 208

### Phenoxazine-10-carboxylic acid [2-(5,8-dihydro-6H-[1,7]naphthyridin-7-yl)ethyl]-amide

Prepared according to the method described in Example 56 with the following modification: triethylamine was used in place of potassium carbonate. HPLC retention time 4.98 min. Mass Spectrum (ES+) m/z 401(M+H).

### Example 209

### 2-(4-Hydroxy-1,3-dihydro-isoindol-2-yl)-1-phenoxazin-10-yl-ethanone

Prepared according to the method described in Example 56. HPLC retention time 3.73 min. Mass Spectrum (ES+) m/z 359(M+H).

### Example 210

### Methanesulphonic acid 2-(2-oxo-2-phenoxazin-10-yl-ethyl)-2,3-dihydro-1H-isoindol-4-yl ester

Prepared according to the method described in Example 124. HPLC retention time 4.18 min. Mass Spectrum (ES+) m/z 437(M+H).

### Example 211

### 1-Carbazol-9-yl-2-(8-methoxy-3,4-dihydro-1H-isoquinolin-2-yl)-ethanone

Prepared according to the method described in Example 56. HPLC retention time 4.86 min. Mass Spectrum (ES+) m/z 371(M+H).

### Example 212

### 2-(8-Methoxy-3,4-dihydro-1H-isoquinolin-2-yl)-1-(3-methyl-2,3-dihydro-benzo[1,4]oxazin-4-yl)-ethanone

Prepared according to the method described in Example 56. HPLC retention time 4.26 min. Mass Spectrum (ES+) m/z 353(M+H).

### Example 213

### 1-(3-tert-Butyl-2,3-dihydro-benzo[1,4]oxazin-4-yl)-2-(8-methoxy-3,4-dihydro-1H-isoquinolin-2-yl)-ethanone

Prepared according to the method described in Example 56. HPLC retention time 4.79 min. Mass Spectrum (ES+) m/z 395(M+H).

### Example 214

### 1-(11H-Dibenzo[b,f][1,4]oxazepin-10-yl)-2-(8-methoxy-3,4-dihydro-1H-isoquinolin-2-yl)-ethanone

Prepared according to the method described in Example 56. HPLC retention time 4.44 min. Mass Spectrum (ES+) m/z 401(M+H).

### Example 215

### 1-(3-Ethyl-2,3-dihydro-benzo[1,4]oxazinyl)-2-(8-methoxy-3,4-dihydro-1H-isoquinolin-2-yl)-ethanone

Prepared according to the method-described in Example 56. HPLC retention time 4.40 min. Mass Spectrum (ES+) m/z 367(M+H).

### Example 216

### 2-(2-Oxo-2-phenoxazin-10-yl-ethyl)-1,2,3,4-tetrahydro-isoquinoline-8-sulphonic acid

Prepared according to the method described in Example 56. HPLC retention time 2.39 min. Mass Spectrum (ES+) m/z 437(M+H).

### Example 217

### N-[2-(2-Oxo-2-phenoxazin-10-yl-ethyl)-2,3-dihydro-1H-isoindol-4-yl]-methanesulphonamide

Prepared according to the method described in Example 124. HPLC retention time 2.86 min. Mass Spectrum (ES+) m/z 436(M+H).

### Example 218

### 1-(3-tert-Butyl-2,3-dihydro-benzo[1,4]oxazin-4-yl)-2-(8-hydroxy-3,4-dihydro-1H-isoquinolin-2-yl)-ethanone

Prepared according to the method described in Example 56. HPLC retention time 4.11 min. Mass Spectrum (ES+) m/z 381(M+H).

### Example 219

### 2-(8-Methoxy-3,4-dihydro-1H-isoquinolin-2-yl)-1-[3-(4-methoxyphenyl)-2,3-dihydro-benzo[1,4]oxazin-4-yl]-ethanone

Prepared according to the method described in Example 56. HPLC retention time 4.51 min. Mass Spectrum (ES+) m/z 445(M+H).

### Example 220

### 1-[3-(2,5-Dimethoxyphenyl)-2,3-dihydro-benzo[1,4]oxazin-4-yl]-2-(8-methoxy-3,4-dihydro-1H-isoquinolin-2-yl)ethanone

Prepared according to the method described in Example 56. HPLC retention time 4.69 min. Mass Spectrum (ES+) m/z 475(M+H).

### Example 221

### N-(4-{4-[2-(8-Methoxy-3,4-dihydro-1H-isoquinolin-2-yl)-acetyl]-3,4-dihydro-2H-benzo[1,4]oxazin-3-yl}phenyl)acetamide

Prepared according to the method described in Example 56. HPLC retention time 3.88 min. Mass Spectrum (ES+) m/z 472(M+H).

### Example 222

### 1-[3-(4-Fluorophenyl)-2,3-dihydro-benzo[1,4]oxazin-4-yl]-2-(8-methoxy-3,4-dihydro-1H-isoquinolin-2-yl)-ethanone

Prepared=according to the method described in Example 56. HPLC retention time 4.57 min. Mass Spectrum (ES+) m/z 433(M+H).

### Example 223

### 1-[3-(3,4-Dimethoxyphenyl)-2,3-dihydro-benzo[1,4]oxazin-4-yl]-2-(8-methoxy-3,4-dihydro-1H-isoquinolin-2-yl)-ethanone

Prepared according to the method described in Example 56. HPLC retention time 4.29 min. Mass Spectrum (ES+) m/z 475(M+H).

Biological Screening

Inhibition of Human Naᵥ1.8 Stably Expressed in SH-SY-5Y Cells

A SH-SY-5Y neuroblastoma cell line stably expressing the human Naᵥ0.8 (hNaᵥ1.8) ion channel was constructed. This cell line has been used to develop a medium to high throughput assay for determining the ability of test compounds to inhibit membrane depolarisation mediated via the hNaᵥ1.8 channel.

SH-SY-5Y hNaᵥ1.8 are grown in adherent monolayer culture using 50:50 Ham's F-12/EMEM tissue culture medium supplemented with 15% (v/v) foetal bovine serum; 2 mM L-glutamine, 1% NEAA and 600 μg·ml⁻¹ Geneticin sulphate. Cells are removed from the tissue culture flask using trypsin/EDTA and re-plated into black walled, clear bottom 96-well assay plates at 50,000 cells·well⁻¹ 24 hours prior to assay.

On the day of assay the cell assay plates are washed to remove cell culture medium using a sodium free assay buffer (145 mM tetramethyl ammonium chloride; 2 mM calcium chloride; 0.8 mM magnesium chloride hexahydrate; 10 mM HEPES; 10 mM glucose; 5 mM potassium chloride, pH 7.4). Fluorescent membrane potential dye solution (FLIPR™ membrane potential dye, Molecular Devices Corporation), containing 10 μM of a pyrethroid to prevent channel inactivation and 250 nM tetrodotoxin (TTX) to reduce interference from TTX-sensitive sodium channels present in the cell line. Test compound, initially dissolved in dimethyl sulfoxide but further diluted in sodium free buffer, is added to achieve the final test concentration range of 100 μM-0.05 μM.

Cell plates are incubated for 30 minutes at room temperature to allow equilibration of dye and test compound. Plates are then transferred to a fluorescence plate reader for fluorescence measurement using an excitation wavelength of 530 nm whilst measuring fluorescence emission at 565 nm. Baseline fluorescence levels are first determined before the addition of a sodium containing buffer (220 mM sodium chloride; 2 mM calcium chloride; 0.8 mM magnesium chloride hexahydrate; 10 nM HEPES; 10 mM glucose; 5 mM potassium chloride. pH 7.4) to cause membrane depolarisation in those cells where channel block has not been effected (final sodium concentration=72.5 mM). Membrane depolarisation is registered by an increase in fluorescence emission at 565 nm.

The change in fluorescence seen in each test well upon the addition of sodium containing buffer is calculated relative to the baseline fluorescence for that well. This figure is then used for calculating the IC₅₀ for each test compound. The results are set out in Tables 1 and 2 below.

TABLE 1

| Compound | IC₅₀ |
|---|---|
| Example 91 | 6.91 |
| Example 92 | 5.27 |
| Example 93 | 4.72 |
| Example 94 | 2.17 |
| Example 95 | 1.71 |
| Example 8 | 0.41 |
| Example 70 | 2.14 |
| Example 48 | 4.84 |
| Example 11 | 0.60 |
| Example 14 | 1.59 |
| Example 16 | 0.88 |
| Example 17 | 1.25 |
| Example 18 | 0.68 |
| Example 19 | 0.73 |
| Example 21 | 1.25 |
| Example 22 | 0.81 |
| Example 23 | 0.26 |
| Example 24 | 1.51 |
| Example 25 | 1.07 |
| Example 26 | 0.67 |
| Example 27 | 1.02 |
| Example 45 | 7.21 |
| Example 32 | 0.23 |
| Example 34 | 0.19 |
| Example 33 | 0.86 |
| Example 35 | 4.86 |
| Example 86 | 1.46 |
| Example 87 | 1.10 |
| Example 88 | 0.58 |
| Example 12 | 0.99 |
| Example 13 | 1.39 |
| Example 9 | 0.43 |
| Example 10 | 0.48 |
| Example 30 | 1.59 |
| Example 29 | 14.96 |
| Example 89 | 0.49 |
| Example 31 | 1.85 |
| Example 47 | 0.47 |
| Example 46 | 0.29 |
| Example 36 | 2.80 |
| Example 38 | 1.39 |
| Example 39 | 0.45 |
| Example 61 | 2.56 |
| Example 62 | 5.63 |
| Example 63 | 15.84 |
| Example 64 | 3.14 |
| Example 65 | 5.64 |
| Example 66 | 2.05 |
| Example 67 | 2.35 |
| Example 68 | 1.95 |
| Example 42 | 1.05 |
| Example 60 | 0.95 |
| Example 40 | 0.97 |
| Example 77 | 0.66 |
| Example 69 | 8.96 |
| Example 41 | 7.02 |
| Example 50 | 2.74 |
| Example 53 | 4.06 |
| Example 52 | 4.68 |
| Example 43 | 1.67 |
| Example 96 | 1.94 |
| Example 97 | 1.06 |

TABLE 2
The compound numbers in Table 2 refer to those set out at pages
21 to 32 of the description.

| Compound | IC₅₀ |
|---|---|
| 90 | 2.05 |
| 71 | 2.18 |
| 72 | 1.29 |
| 73 | 1.28 |
| 74 | 1.80 |
| 76 | 14.75 |
| 78 | 28.52 |
| 79 | 1.02 |
| 80 | 0.00 |
| 81 | 1.40 |
| 82 | 1.06 |
| 83 | 0.83 |
| 84 | 0.40 |
| 85 | 1.08 |
| 86 | 0.49 |
| 87 | 0.49 |
| 88 | 2.09 |
| 91 | 6.98 |
| 92 | 4.82 |
| 94 | 2.84 |
| 95 | 0.53 |
| 96 | 1.11 |
| 97 | 2.27 |
| 98 | 6.76 |
| 99 | 2.43 |
| 100 | 0.97 |
| 101 | 4.26 |
| 102 | 0.86 |
| 103 | 2.25 |
| 105 | 15.89 |
| 107 | 2.26 |
| 108 | 3.48 |
| 109 | 1.80 |
| 110 | 0.48 |
| 111 | 3.88 |
| 112 | 0.79 |
| 114 | 10.62 |
| 115 | 2.35 |
| 116 | 22.19 |
| 117 | 1.36 |
| 118 | 10.23 |
| 119 | 1.91 |
| 120 | 3.34 |
| 121 | 3.45 |
| 122 | 0.96 |
| 123 | 2.89 |
| 124 | 0.59 |
| 125 | 0.38 |
| 126 | 1.93 |
| 127 | 0.65 |
| 128 | 3.51 |
| 129 | 2.48 |
| 131 | 3.61 |
| 132 | 0.49 |
| 133 | 0.60 |
| 134 | 0.77 |
| 135 | 0.38 |
| 136 | 2.11 |
| 137 | 0.44 |
| 138 | 0.52 |
| 139 | 1.25 |
| 140 | 2.33 |
| 141 | 1.85 |
| 142 | 2.09 |
| 143 | 9.59 |
| 144 | 0.50 |
| 145 | 2.63 |
| 147 | 2.04 |
| 148 | 5.17 |
| 149 | 9.09 |
| 150 | 1.79 |
| 151 | 7.44 |
| 152 | 3.03 |
| 153 | 5.31 |
| 154 | 12.40 |
| 155 | 6.26 |
| 156 | 2.37 |
| 157 | 30.00 |
| 158 | 4.59 |
| 159 | 20.78 |
| 160 | 0.95 |
| 161 | 1.11 |
| 162 | 0.97 |
| 163 | 0.77 |
| 164 | 0.71 |
| 165 | 1.02 |
| 166 | 0.35 |
| 167 | 4.51 |
| 168 | 2.01 |
| 169 | 29.90 |
| 170 | 1.69 |
| 171 | 1.75 |
| 172 | 6.27 |
| 173 | 5.64 |
| 174 | 0.55 |
| 175 | 1.53 |
| 176 | 5.95 |
| 178 | 0.62 |
| 180 | 5.19 |
| 181 | 20.35 |
| 182 | 2.14 |
| 183 | 2.26 |
| 184 | 10.88 |
| 185 | 1.85 |
| 186 | 29.13 |
| 187 | 19.06 |
| 188 | 12.05 |
| 189 | 0.51 |
| 190 | 0.53 |
| 191 | 1.27 |
| 192 | 9.06 |
| 193 | 3.17 |
| 194 | 2.89 |
| 195 | 1.85 |
| 196 | 0.93 |
| 197 | 3.83 |
| 198 | 1.83 |
| 199 | 8.26 |
| 200 | 1.66 |
| 201 | 6.09 |
| 202 | 0.68 |
| 203 | 1.11 |
| 204 | 10.67 |
| 205 | 4.63 |
| 206 | 1.62 |
| 207 | 0.94 |
| 208 | 1.68 |
| 210 | 3.46 |
| 211 | 3.03 |
| 212 | 5.06 |
| 213 | 8.24 |
| 214 | 6.36 |
| 215 | 1.11 |
| 216 | 4.31 |
| 217 | 1.96 |
| 218 | 12.85 |
| 219 | 0.8 |
| 220 | 0.8 |

1. Use of a compound of the formula (I), or a pharmaceutically acceptable salt thereof in the manufacture of a medicament for use in the treatment or prevention of a condition involving sodium ion flux through a sensory neurone specific channel of a sensory neurone 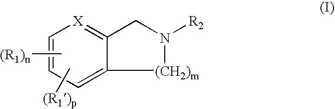
wherein:
X is —N— or —CH—;
n is from 0 to 3;
each R₁ is the same or different and is a hydroxy, amino, halogen, C₁-C₆ alkyl, C₁-C₆ haloalkyl, C₁-C₆ alkoxy, C₂-C₆ alkenyloxy, C₂-C₆ alkynyloxy, C₁-C₆ haloalkoxy, C₁-C₆ alkylthio, C₁-C₆ haloalkylthio, (C₁-C₆ alkyl) amino or di(C₁-C₆ alkyl) amino group;
p is 0 or 1;
R₁¹ is cyano, —NR₁—CO—(C₁-C₄ alkyl), —NR₁—S(O)₂—(C₁-C₄ alkyl), —CO₂H, —S(O)₂OH, —CO₂—(C₁-C₄ alkyl), —O—S(O)₂—(C₁-C₄ alkyl) or —N[S(O)₂—(C₁-C₄ alkyl)]₂, wherein R₁ is hydrogen or a C₁-C₄ alkyl group;
m is 1, 2 or 3; and
R₂ is either
(a) -L-A, wherein L is a direct bond or a C₁-C₆ alkyl, C₂-C₆ alkenyl or C₂-C₆ alkynyl moiety and A is C₆-C₁₀ aryl, C₃-C₆ carbocyclyl, a 5- to 10-membered heteroaryl group or a 5- to 10-membered heterocyclic group,
(b) -L-CR(A)₂ or -L-CH═C(A)₂ wherein R is hydrogen or C₁-C₄ alkyl, L is as defined above and each A is the same or different and is as defined above,
(c) -L¹-Het-A¹, wherein Het is —O—, —S— or —NR¹—, A¹ is -L-A, -L-CR(A)₂ or -L-CH═C (A)₂, R¹ is H or -L-A, L¹ is a C₁-C₆ alkyl, C₂-C₆ alkenyl or C₂-C₆ alkynyl moiety, L is as defined above, R is as defined above and each A is the same or different and is as defined above,
(d) -L-CO—NR₃R₄ or -L-CS—NR₃R₄, wherein L is as defined above and either (i) R₃ and R₄, together with the N atom to which they are attached, form a 5- to 10-membered heteroaryl or heterocyclyl group or (ii) R₃ represents -L-H or A¹ wherein L and A¹ are as defined above, and R₄ represents -L¹-H, -L¹-CO-A¹, -L¹-S(O)-A¹, -L¹-S(O)₂-A¹, -L¹-Het-A¹, —NR—CO—N(A)₂, —N(A)₂, -A-Het-A, -A¹, -L-CR(LA)₂ or -L-CH═C(LA)₂ wherein each L is the same or different, each A is the same or different, and L¹, L, R, A and A¹ are as defined above,
(e) —CO-L-NR₃R₄ or —CS-L-NR₃R₄ wherein L, R₃ and R₄ are as defined above, (f) —CO-A¹ or —CS-A¹ wherein A¹ is as defined above,
(g) -L¹-O—N═C(A)₂ or —CO-L¹-O—N═C(A)₂ wherein L¹ is as defined above and each A is the same or different and is as defined above, or
(h) -L¹-NR—CO—NR₃R₄ or -L¹-NR—CS—NR₃R₄, wherein L¹, R, R₃ and R₄ are as defined above,
wherein
said aryl, carbocyclyl, heteroaryl and heterocyclyl groups are optionally fused to one or two cyclic moieties selected from phenyl rings and 5- to 6-membered heterocyclyl and heteroaryl groups, and
said aryl, heteroaryl, carbocyclyl and heterocyclyl groups are unsubstituted or are substituted by 1, 2 or 3 substituents which are the same or different and are selected from C₁-C₄ alkyl, C₁-C₄ haloalkyl, halogen, hydroxy, amino, (C₁-C₄ alkyl)amino, di(C₁-C₄ alkyl)amino, C₁-C₄ alkoxy, C₁-C₄ haloalkoxy, C₁-C₄ alkylthio, C₁-C₄ haloalkylthio, —NH—CO—(C₁-C₄ alkyl), —CO—(C₁-C₄) alkyl, —CO₂—(C₁-C₄ alkyl), 5- or 6-membered heteroaryl, phenyl and —CHPh₂ substituents, the phenyl and heteroaryl moieties in said substituents being unsubstituted or substituted by 1 or 2 further substituents selected from halogen atoms, C₁-C₂ alkyl groups, C₁-C₂ alkoxy groups and —NH—CO—(C₁-C₂ alkyl) groups,
provided that (a) when R₂ is -L-A, A is other than a benzimidazolyl group, and (b) when R₂ is —CO-A¹ or —CS-A¹, A is other than a pyrazolopyrimidinyl or pyrazolyl group.

2. Use according to claim 1, wherein:
X is —N— or —CH—;
n is from 0 to 3;
p is 0;
each R₁ is the same or different and is a hydroxy, amino, halogen, C₁-C₆ alkyl, C₁-C₆ haloalkyl, C₁-C₆ alkoxy, C₁-C₆ haloalkoxy, C₁-C₆ alkylthio, C₁-C₆ haloalkylthio, (C₁-C₆ alkyl)amino or di(C₁-C₆ alkyl)amino group;
m is 1, 2 or 3; and
R₂ is either
(a) -L-A, wherein L is a direct bond or a C₁-C₆ alkyl, C₂-C₆ alkenyl or C₂-C₆ alkynyl moiety and A is C₆-C₁₀ aryl, C₃-C₆ carbocyclyl, a 5- to 10-membered heteroaryl group or a 5- to 10-membered heterocyclic group,
(b) -L-CR(A)₂ or -L-CH═C(A)₂ wherein R is hydrogen or C₁-C₄ alkyl, L is as defined above and each A is the same or different and is as defined above,
(c) -L¹-Het-A¹, wherein Het is —O—, —S— or —NR¹—, A¹ is -L-A, -L-CR(A)₂ or -L-CH═C (A)₂, R¹ is H or -L-A, L1 is a C₁-C₆ alkyl, C₂-C₆ alkenyl or C₂-C₆ alkynyl moiety, L is as defined above, R is as defined above and each A is the same or different and is as defined above,
(d) -L-CO—NR₃R₄ or -L-CS—NR₃R₄, wherein L is as defined above and either (i) R₃ and R₄, together with the N atom to which they are attached, form a 5- to 10-membered heteroaryl or heterocyclyl group or (ii) R₃ represents -L-H or A¹ wherein L and A¹ are as defined above, and R₄ represents -L¹-H, -L¹-CO-A, A¹, -L-CR(LA)₂ or -L-CH═C(LA)₂ wherein each L is the same or different, each A is the same or different, and L¹, L, R, A and A¹ are as defined above,
(e) —CO-L-NR₃R₄ or —CS-L-NR₃R₄ wherein L, R₃ and R₄ are as defined above, (f) —CO-A¹ or —CS-A¹ wherein A¹ is as defined above, or
(g) -L-O—N═C(A)₂ or —CO-L-O—N═C(A)₂ wherein L is as defined above and each A is the same or different and is as defined above,
wherein
said aryl, carbocyclyl, heteroaryl and heterocyclyl groups are optionally fused to one or two cyclic moieties selected from phenyl rings and 5- to 6-membered heterocyclyl and heteroaryl groups, and
said aryl, heteroaryl, carbocyclyl and heterocyclyl groups are unsubstituted or are substituted by 1, 2 or 3 substituents which are the same or different and are selected from C₁-C₄ alkyl, C₁-C₄ haloalkyl, halogen, hydroxy, C₁-C₄ alkoxy, C₁-C₄ haloalkoxy, C₁-C₄ alkylthio, C₁-C₄ haloalkylthio, phenyl and —CHPh2 substituents, the phenyl moieties in said substituents being unsubstituted or substituted by 1 or 2 halogen atoms,
provided that (a) when R₂ is -L-A, A is other than a benzimidazolyl group and (b) when R₂ is —CO-A¹ or —CS-A¹, A is other than a pyrazolopyrimidinyl or pyrazolyl group.

3. Use according to claim 1, wherein the aryl, heteroaryl, heterocyclyl and carbocyclyl groups and moieties in the substituents R₁, R₂, R₃ and R₄ are unsubstituted or substituted by 1, 2 or 3 substituents which are the same or different and are selected from halogen, C₁-C₄ alkyl, hydroxy, amino, (C₁-C₄ alkyl) amino, C₁-C₄ alkoxy, C₁-C₄ haloalkyl, C₁-C₄ haloalkoxy, C₁-C₄ alkylthio, C₁-C₄ haloalkylthio, —NH—CO—(C₁-C₂ alkyl), —CO—(C₁-C₂ alkyl), —CO₂—(C₁-C₂ alkyl), 5-membered heteroaryl, phenyl and —CHPh₂ substituents, the phenyl and heteroaryl moieties in said substituents being unsubstituted or substituted by one or two further substituents selected from halogen atom, C₁-C₂ alkyl groups, C₁-C₂ alkoxy groups and —NH—CO—(C₁-C₂ alkyl) groups.

4. Use according to claim 1, wherein each R₁ is the same or different and is a hydroxy, amino, halogen, C₁-C₄ alkyl, C₁-C₄ haloalkyl, C₁-C₄ alkoxy, C₂-C₄ alkenyloxy, C₁-C₄ haloalkoxy, C₁-C₄ alkylthio or C₁-C₄ haloalkylthio group.

5. Use according to claim 1, wherein each L moiety in the R₂ substituent is the same or different and represents a direct bond or a C₁-C₄ alkyl moiety and/or each L¹ moiety in the R₂ substituent is the same or different and represents a C₁-C₄ alkyl moiety.

6. Use according to claim 1, wherein each A moiety in the R₂ substituent is the same or different and represents a C₆-C₁₀ aryl, C₃-C₆ cycloalkyl, 5- or 6-membered heterocyclyl or 5- or 6-membered heteroaryl group, which group is (a) unsubstituted or substituted by 1, 2 or 3 substituents selected from C₁-C₄ alkyl, C₁-C₄ haloalkyl, halogen, hydroxy, amino, (C₁-C₄ alkyl)amino, di(C₁-C₄alkyl)amino, C₁-C₄ alkoxy, C₁-C₄ haloalkoxy, C₁-C₄ alkylthio, C₁-C₄ haloalkylthio, —NH—CO—(C₁-C₂ alkyl), phenyl and halophenyl substituents and (b) optionally fused to one or two cyclic moieties selected from phenyl rings and 5- to 6-membered heterocyclyl or heteroaryl groups.

7. Use according to claim 1, wherein each R substituent in each —CR(A)₂ moiety is the same or different and is hydrogen or methyl.

8. Use according to claim 1, wherein each Het moiety in the R₂ substituent is —O—, —S— or —NR— wherein R is hydrogen, C₁-C₄ alkyl, phenyl or —(C₁-C₄ alkyl)-phenyl.

9. Use according to claim 1, wherein, when R₃ and R₄, together with the nitrogen atom to which they are attached, form a heterocycle, they form a 5- to 7-membered heterocyclyl group.

10. Use according to claim 9, wherein, when R₃ and R₄, together with the nitrogen atom to which they are attached, form a heterocycle, they form a morpholino, thiomorpholino, S-oxo-thiomorpholino, S,S-dioxo-thiomorpholino, pyrrolidinyl, piperazinyl or homopiperidinyl ring which is (a) optionally fused to one or two cyclic moieties selected from phenyl rings and 5- to 6-membered heteroaryl rings, and (b) unsubstituted or substituted by 1 or 2 substituents selected from C₁-C₄ alkyl, C₁-C₄ haloalkyl, C₁-C₄ alkoxy, C₁-C₄ alkylthio, halogen, phenyl, —CHPh₂, —CO—(C₁-C₂ alkyl), —CO₂—(C₁-C₂ alkyl) and 5- to 6-membered heteroaryl substituents, the phenyl and heteroaryl moieties in said substituents being unsubstituted or substituted by 1 or 2 further substituents selected from halogen atoms, C₁-C₂ alkyl groups, C₁-C₂ alkoxy groups and —NH—CO(C₁-C₂ alkyl) groups.

11. Use according to any claim 1, wherein, when R₃ and R₄ do not together form a heterocycle, R₃ represents hydrogen or a C₁-C₄ alkyl, phenyl, —(C₁-C₄ alkyl)-phenyl or —(C₁-C₄ alkyl)-CHPh₂ group in which the phenyl moieties are unsubstituted or substituted by a hydroxy group and R₄ represents C₁-C₄ alkyl, A, —(C₁-C₄alkyl)-A, —(CH₂)ₘ—CH(A)₂, —CH[(CH₂)ₘA]₂, —(CH₂)ₘ—CO-A, —(CH₂)ₘ—O—CH(A)₂, —(CH₂)ₘ—S—CH(A)₂, —(CH₂)ₘ—S(O)—CH A)₂, —(CH₂)ₘ—S(O)₂—CH(A)₂, —NH—CO—N(A)₂, —N(A)₂ or -A-O-A, wherein each A is the same or different and is as defined above and m is 0, 1, 2, 3 or 4, the A moieties in the R₄ substituent being (a) unsubstituted or substituted by one or two substituents selected from C₁-C₄ alkyl, C₁-C₄ alkoxy, halogen, hydroxy, amino, C₁-C₂ haloalkyl, C₁-C₂ haloalkoxy and C₁-C₂ haloalkylthio substituents and (b) monocyclic or fused to one or two phenyl rings.

12. Use according to any claim 1, wherein, when R₂ is defined according to option (a), A is monocyclic.

13. Use according to claim 1, wherein, when R₂ is defined according to option (f), A is a said C₆-C₁₀ aryl group.

14. Use according to claim 1, wherein
X is —N— or —CH—;
n is 0 or 1;
each R₁ is the same or different and is C₁-C₂ alkyl, hydroxy or C₁-C₂ alkoxy;
p is 0 or 1;
R₁¹ is cyano, —NH—CO—CH₃, —NH—S(O)₂—CH₃, —O—S(O)₂—CH₃, —N[SO₂—CH₃]₂ or —S(O)₂—OH;
m is 1, 2 or 3; and
R₂ is either
(a) -L-A wherein L represents a direct bond or a C₁-C₄ alkyl moiety, for example a methyl, ethyl or propyl moiety, and A is a phenyl, thienyl, triazolyl, pyridyl, fluorenyl, thiazolyl, tetrahydroisoquinolinyl, 9H-carbazolyl, indolinyl, 9H-xanthenyl or benzimidazolyl group, which group is unsubstituted or substituted by one or two substituents selected from halogen, C₁-C₂ alkyl, hydroxy, amino, C₁-C₂ alkoxy, C₁-C₂ haloalkyl, C₁-C₂ haloalkoxy, C₁-C₂ haloalkylthio, —NH—CO—CH₃ and phenyl substituents,
(b) -L-CR(A) 2 or -L-CH═C(A)₂ wherein R is hydrogen or methyl, L is as defined above and each A is the same or different and is as defined above,
(c) -L¹-Het-A¹ wherein Het is —O— or —NR¹— wherein R¹ is hydrogen, C₁-C₄ alkyl or benzyl, A¹ is -L-A, -L-CR(A)₂ or -L-CH═C(A)₂, L¹ is a C₁-C₄ alkyl moiety, for example a methyl, ethyl or propyl moiety, L is as defined above, R is as defined above and each A is the same or different and is as defined above,
(d) -L-CO—NR₃R₄ wherein L is as defined above and either (i) R₃ and R₄, together with the nitrogen atom to which they are attached, form a morpholino, thiomorpholino, S-oxo-thiomorpholino, S,S-dioxo-thiomorpholino, pyrrolidinyl, piperazinyl or homopiperidinyl ring which is (a) optionally fused to one or two cyclic moieties selected from phenyl rings and 5- to 6-membered heteroaryl rings, and (b) unsubstituted or substituted by one or two substituents selected from C₁-C₄ alkyl, C₁-C₄ haloalkyl, C₁-C₄ alkoxy, C₁-C₄ alkylthio, halogen, phenyl, —CHPh₂, —CO—(C₁-C₂ alkyl), —CO₂—(C₁-C₂ alkyl) and 5- to 6-membered heteroaryl substituents, the phenyl and heteroaryl moieties in said substituents being unsubstituted or substituted by one or two further substituents selected from halogen atoms, C₁-C₂ alkyl groups, C₁-C₂ alkoxy groups and —NH—CO—(C₁-C₂ alkyl) groups, or (ii) R₃ represents hydrogen, C₁-C₄ alkyl or an unsubstituted benzyl, phenyl, hydroxyphenyl or —(C₁-C₂ alkyl)-CHPh₂ group and R₄ represents-C₁-C₄ alkyl, fluorenyl, phenyl, pyridyl, (C₁-C₄ alkyl)-phenyl, —(C₁-C₄ alkyl)-(5- to 6-membered heteroaryl), —(CH₂)ₘ-(9H-carbazolyl), —(CH₂)ₘ-indolinyl, —(CH₂)ₘ-(9H-xanthenyl), —(CH₂)ₘ—O—CHA¹¹A¹¹¹, —(CH₂)ₘ—S—CHA¹¹A¹¹¹, —(CH₂)ₘ—S(O)—CHA¹¹A¹¹¹, —(CH₂)ₘ—S(O)₂—CHA¹¹A¹¹¹, —NH—CO—N(phenyl)₂, —N(phenyl)₂ or -A¹¹-O-A¹¹¹, —(CH₂)ₘ—CHA¹¹A¹¹¹-CH[(CH₂)ₙPh]₂ or —(CH₂)ₚ—CO—R where m is 0, 1, 2 or 3, A¹¹ and A¹¹¹ are the same or different and each represent phenyl or a 5- or 6-membered heteroaryl group, n is 0, 1 or 2, p is 1, 2 or 3 and R is 5- or 6-membered heterocyclic group fused to a phenyl ring, for example a-tetrahydroisoquinoline group, the cyclic moieties in said R₄ groups being unsubstituted or substituted by a halogen atom, C₁-C₂ alkyl, hydroxy, amino or C₁-C₂ alkoxy group,
(e) —CO-L-NR₃R₄ or —CS-L-NR₃R₄ wherein L, R₃ and R₄ are as defined above, (f) —CO-A¹ or —CS-A¹ where A¹ is as defined above,
(g) —CO-L¹-O—N═C(A)₂ wherein L¹ is as defined above and each A is the same or different and is as defined above; or
(h) -L¹-NR—CO—NR₃R₄ or -L¹-NR—CS—NR₃R₄ wherein L¹, R, R3 and R4 are as defined above,
provided that when R₂ is -L-A, A is monocyclic.

15. Use according to claim 1, wherein said condition is chronic or acute pain, a bowel disorder, a bladder dysfunction, tinnitus or a demyelinating disease.

16. A compound of the formula (I), as defined in claim 1, or a pharmaceutically acceptable salt thereof.

17. A pharmaceutical composition comprising a compound of the formula (I), as defined in claim 1, or a pharmaceutically acceptable salt thereof, and a pharmaceutically acceptable carrier or diluent.

18. A composition according to claim 17 which is a capsule or tablet comprising from 10 to 500 mg of a compound of the formula (I), as defined in any one of claims 1 to 14, or a pharmaceutically acceptable salt thereof.

19. An inhalation device comprising a pharmaceutical composition according to claim 18.

20. An inhalation device according to claim 19 which is a nebulizer.

21. A compound according to claim 1, or a pharmaceutically acceptable salt thereof, for use in the treatment of the human or animal body.

22. A method of treating a patient suffering from or susceptible to a condition as defined in claim 1, which method comprises administering to said patient an effective amount of a compound of formula (I), or a pharmaceutically acceptable salt thereof.
